(19) United States
(12) Patent Application Publication
(10) Pub. No.: US 2021/0220358 A1
(43) Pub. Date: Jul. 22, 2021

(54) 1,3-THIAZOL-2-YL SUBSTITUTED BENZAMIDES FOR THE TREATMENT OF DISEASES ASSOCIATED WITH NERVE FIBER SENSITIZATION

(71) Applicants: Bayer Aktiengesellschaft, Leverkusen (DE); Bayer Pharma Aktiengesellschaft, Berlin (DE)

(72) Inventors: Christian FRIEDRICH, Brandenburg (DE); Isabella GASHAW, Berlin (DE); Damian BROCKSCHNIEDER, Haan (DE); Oliver Martin FISCHER, Berlin (DE)

(73) Assignees: Bayer Aktiengesellschaft, Leverkusen (DE); Bayer Pharma Aktiengesellschaft, Berlin (DE)

(21) Appl. No.: 17/055,488

(22) PCT Filed: May 14, 2019
(86) PCT No.: PCT/EP2019/062332

§ 371 Date: Nov. 13, 2020

(30) Foreign Application Priority Data
May 15, 2018 (EP) 18172409.7

(51) Int. Cl. A61K 31/506; A61K 31/5377; A61K 9/00; A61P 11/14; A61P 11/06; A61P 11/00

(52) U.S. Cl. CPC A61K 31/506; A61K 31/5377; A61P 11/00; A61P 11/14; A61P 11/06; A61K 9/0053

(57) ABSTRACT

The present invention relates to use 1,3-thiazol-2-yl substituted benzamide compounds of general formula (I) as described and defined herein, to pharmaceutical compositions and combinations comprising said compounds for the treatment or prophylaxis of diseases which are associated with nerve fibre sensitization, in particular for treatment and prophylaxis of Chronic Cough (CC), Idiopathic Pulmonary Fibrosis (IPF), Chronic Obstructive Pulmonary Disorder (COPD), and asthma.

The present invention relates to the use of 1,3-thiazol-2-yl substituted benzamide compounds of general formula (I) as a sole agent or in combination with other active ingredients as well as to the use of pharmaceutical compositions and combinations comprising said compounds for the treatment or prophylaxis of diseases which are associated with nerve fibre sensitization, in particular Chronic Cough.

### BACKGROUND OF THE INVENTION

The present invention relates to the use of chemical compounds that inhibit P2X3 receptor for the treatment of diseases associated with nerve fiber sensitization. P2X purinoceptor 3 is a protein that in humans is encoded by the P2RX3 gene (Garcia-Guzman M, Stuehmer W, Soto F, 1997, Brain Res Mol Brain Res 47 (1-2): 59-66). The product of this gene belongs to the family of purinoceptors for ATP. This receptor functions as a ligand-gated ion channel and transduces ATP-evoked nociceptor activation.

P2X purinoreceptors are a family of ligand-gated ion channels that are activated by ATP. To date, seven members of this family have been cloned, comprising P2X1-7 (Burnstock 2013, front Cell Neurosci 7:227). These channels can exist as homomers and heteromers (Saul 2013, front Cell Neurosci 7:250). Purines, such as ATP, have been recognized as important neurotransmitters and by acting via their respective receptors they have been implicated in various physiological and pathophysiological roles (Burnstock 1993, Drug Dev Res 28:196-206; Burnstock 2011, Prog Neurobiol 95:229-274; Jiang 2012, Cell Health Cytoskeleton 4:83-101).

Among the P2X family members, in particular the P2X3 receptor has been recognized as an important mediator of nociception (Burnstock 2013, Eur J Pharmacol 716:24-40; North 2003, J Phyiol 554:301-308; Chizh 2000, Pharmacol Rev 53:553-568). It is mainly expressed in dorsal root ganglia in a subset of nociceptive sensory neurons. During inflammation the expression of the P2X3 receptor is increased, and activation of P2X3 receptor has been described to sensitize peripheral nerves (Fabretti 2013, front Cell Neurosci 7:236).

The prominent role of the P2X3 receptor in nociception has been described in various animal models, including mouse and rat models for acute, chronic and inflammatory pain. P2X3 receptor knock-out mice show a reduced pain response (Cockayne 2000, Nature 407:1011-1015; Souslova 2000, Nature 407:1015-1017). P2X3 receptor antagonists have been shown to act anti-nociceptive in different models of pain and inflammatory pain (Ford 2012, Purin Signal 8 (Suppl 1):S3-S26). The P2X3 receptor has also been shown to integrate different nociceptive stimuli. Hyperalgesia induced by PGE2, ET-1 and dopamine have all been shown to be mediated via release of ATP and activation of the P2X3 receptor (Prado 2013, Neuropharm 67:252-258; Joseph 2013, Neurosci 232C: 83-89).

Besides its prominent role in nociception and in pain-related diseases involving both chronic and acute pain, the P2X3 receptor has been shown to be involved in genitourinary, gastrointestinal, cardiovascular and respiratory conditions and disorders, including overactive bladder, Chronic Cough, heart failure and hypertension (Ford 2013, front Cell Neurosci 7:267; Burnstock 2014, Purin Signal 10(1):3-50; Pijacka et al, Nat Med. 2016. 22(10): 1151-1159). Therein, sensitization of nerve fibers has been recognized as a mechanism that can lead to activation of afferent nerve fiber signaling also below levels required for the stimulation of physiological signaling processes. As examples, P2X3 receptor expression has been shown to be elevated in pathophysiological situations, or P2X3 has been shown to be phosphorylated thereby changing its activity (Ford 2013, front Cell Neurosci 7:267; Burnstock 2014, Purin Signal 10(1):3-50). ATP-release can occur in these examples e.g. from epithelial cells, which in turn activates the P2X3 receptor finally leading to e.g. the contraction of lung muscles after central processing of afferent signals, thereby inducing cough.

P2X3 subunits do not only form homotrimers but also heterotrimers with P2X2 subunits. P2X3 subunits and P2X2 subunits are also expressed on nerve fibres innervating the tongue, therein taste buds (Kinnamon 2013, front Cell Neurosci 7:264). In a physiological setting, receptors containing P2X3 and/or P2X2 subunits are involved in the transmission of taste from the tongue (bitter, sweet, salty, umami and sour). Recent data show that while blocking the P2X3 homomeric receptor alone is important to achieve anti-nociceptive efficacy, non-selective blockade of both the P2X3 homomeric receptor and the P2X2/3 heteromeric receptor leads to changes in taste perception which might limit the therapeutic use of non-selective P2X3 and P2X2/3 receptor antagonists (Ford 2014, purines 2014, abstract book p 15). Therefore, compounds that differentiate between P2X3 and P2X2/3 receptors are highly desirable.

Compounds blocking both the exclusively P2X3 subunit containing ion channel (P2X3 homomer) as well as the ion channel composed of P2X2 and P2X3 subunit (P2X2/3 heterotrimer) are called P2X3 and P2X2/3 nonselective receptor antagonists (Ford, Pain Manag 2012, 2(3), 267-77). Clinical Phase II trials demonstrated that AF-219, a P2X3 antagonist, leads to taste disturbances in treated subjects by affecting taste sensation via the tongue (e.g. Abdulqawi et al, Lancet 2015, 385 (9974), 1198-1205; Strand et al, 2015 ACR/ARMP Annual Meeting, Abstract 2240). This side effect has been attributed to the blockade of P2X2/3 channels, i.e. the heterotrimer (A. Ford, London 2015 Pain Therapeutics Conference, congress report). Both P2X2 and P2X3 subunits are expressed on sensory nerve fibers innervating the tongue. Knock-out animals deficient for P2X2 and P2X3 subunits show reduced taste sensation and even taste loss (Finger et al, Science 2005, 310 (5753), 1495-99), whereas P2X3 subunit single knock-outs exhibit a mild or no change in phenotype with respect to taste. Moreover, two distinct populations of neurons have been described in the geniculate ganglion expressing either P2X2 and P2X3 subunits or P2X3 subunit alone (Vandenbeuch et al, J Physiol. 2015, 593(Pt 5): 1113-1125). The population expressing P2X2/P2X3 heterotrimers has been described as being less sensitive to a non-selective P2X2/P2X3 antagonist compared to the population expressing P2X3 homomers, i.e. requiring a higher concentration of this antagonist to be inhibited. In an in vivo setting assessing taste preference towards an artificial sweetener via a lickometer in this study, only at very high free plasma levels (>100 μM) effects on taste were observed, indicating that the less sensitive P2X2 and P2X3 subunits expressing population plays a more important role in taste sensation than the P2X3 subunit expressing population (Vandenbeuch et al, J Physiol. 2015, 593(Pt 5): 1113-1125). Hence, as a modified taste perception has profound effects on the quality of life of patients, P2X3-homomeric receptor-selective antagonists are deemed to be superior towards non-selective receptor antagonists and are considered to represent a solution towards the problem of insufficient patient compliance during chronic treatment as indicated by increased drop-out rates during Phil trials (Strand et a, 2015 ACR/ARMP Annual Meeting, Abstract 2240 and A. Ford, London 2015 Pain Therapeutics Conference, congress report).

A cough is a sudden and often repetitively occurring reflex, which helps to clear the large breathing passages from secretions, irritants, foreign particles, and microbes. A cough can be classified by its duration, character, quality, and timing. The duration can be either acute (of sudden onset) if it is present less than three weeks, subacute if it is present between three or eight weeks, and chronic when lasting longer than eight weeks.

In general Chronic Cough (CC) is defined as cough lasting more than eight weeks or longer. According to literature (see for example P. Gibson et al., CHEST Journal 2016, Vol 149 Iss. 1 p. 27-44), CC is broadly divided into
chronic cough secondary to an underlying pathology where treatment of this pathology also cures the cough.
Idiopathic Chronic Cough (ICC), when patients have no identified causes of chronic cough. ICC is also called Unexplained Chronic Cough (UCC) in the sense of a synonym. If therapy with drugs commonly used for the treatment of cough has been tried in patients where no cause of cough could be identified (empiric therapy), said patient subgroup suffers from unexplained and refractory chronic cough.
Refractory Chronic Cough (RCC), when cough persists after diagnosis and treatment of cough related conditions (such as gastroesophageal reflux disease, asthma; Gibson et al, BMJ 2015, 351:h5590).

RCC as well as ICC or UCC refer to chronic cough which is also characterized in that there are no hallmarks to define and diagnose it, in contrast to other respiratory diseases (e.g. COPD), i.e. CC is currently a diagnosis of exclusion.

Another characteristic of Chronic Cough is that a subject suffering from Chronic Cough may be apparently normal in most other respiratory parameters, but often represent with comorbidities due to the CC like depression, urinary incontinence, sleep abnormalities, and anxiety. Frequent coughing and bothersome coughing during sleep characterize Chronic Cough. There is no lower limit for the cough frequency in patients with Chronic Cough; patients included in recent clinical trials showed cough frequencies in the range of about 30 to 70 coughs/hour. However, patients with lower cough frequency, for example 5 coughs/hour, are also qualified for treatment (Abdulqawi et al, Lancet 2015, 385 (9974), 1198-1205; Ryan et al., Lancet 2012, 380, 1583-89). Chronic Cough can last for a period of years, including over a decade.

In order to determine if a subject, in which a secondary cough due factors like smoking, COPD or cancer has been excluded, is afflicted by a Chronic Cough, in particular of ICC and RCC, a practitioner or clinician can perform a three-step test. First, the subject can be treated for putative post-nasal drips. In some cases, such treatment takes the form of an antihistamine. Second, the subject can be treated with a proton-pump inhibitor (e.g., to treat putative gastro-esophageal disease such as reflux disease). Third, a subject can be treated with steroids (e.g., to treat a putative case of asthma).

If a subject continues to display a Chronic Cough after the above three-step treatment regimen, the cough is said to be refractory or idiopathic chronic cough.

Chronic Cough has a substantial impact on quality of life of patients (Ford et al, Thorax 2006, 61(11):975-9; French et al, Arch Intern Med 1998, 158(15):1657-61). Recent research has highlighted the role of neuronal hypersensitivity of afferent nerves in the pathophysiology of CC.

At present, there are no approved treatment options available for Chronic Cough. Treatment guidelines such as the ACCP guideline (American College of Chest Physicians) identified four treatment categories that were supported by randomized controlled trials: non-pharmacologic therapies such as speech pathology treatment, amongst off-label use of inhaled corticosteroids, neuromodulators, and other therapeutics such as proton pump inhibitors in the case of gastroesophageal reflux disease (GERD). At present, the use of speech therapy is recommended before medical treatments were started according to the ACCP guidance. Inhaled corticosteroids are effective in treating eosinophilic airway inflammation. RCC and ICC caused by neuronal hypersensitivity is treated at present using centrally acting drugs like neuromodulators, for example gabapentin, pregabalin, amitriptyline and baclofen, as well as centrally acting drugs like opioids, for example morphine, codeine or pholcodine. While these agents improve cough specific quality of life in patients, adverse effects can be serious and limit the maximum tolerable dose of these agents (Gibson et al, BMJ 2015, 351:h5590). Severe side effects such as drowsiness, nausea, constipation, sedation, and physical dependence were reported.

WO2015/027212 (Afferent Pharmaceutical Inc.) discloses new diaminopyrimidine compounds having activity as antagonists of P2X purinergic receptors, and methods for treatment of diseases associated with P2X receptors comprising administration of an effective amount of a diaminopyrimidine compound. More particularly, methods are provided for using P2X3 and/or P2X2/3 antagonists in the treatment of cough, Chronic Cough and urge to cough in respiratory conditions and disorders.

Afferent Pharmaceuticals is developing AF-219 (5-(2,4-diamino-pyrimidin-5-yloxy)-4-isopropyl-2-methoxy-benzenesulfonamide), which is an oral, small molecule P2X3 antagonist, for the potential treatment of Chronic Cough and pain, including chronic bladder pain syndrome and osteoarthritis pain, and asthma. Several clinical trials are ongoing, amongst them for example an US phase II trial in patients with idiopathic pulmonary fibrosis with persistent cough and breathlessness (ClinicalTrials.gov Identifier: NCT02502097)) as well as a phase IIb trial in patients with refractory chronic cough (NCT02349425) which are completed.

It is concluded that the state of the art does not describe any approved treatment options for Chronic Cough (CC) which can be used as long-term oral therapy. In addition, known treatment approaches for Chronic Cough as well as for other chronic upper respiratory diseases like Idiopathic Pulmonary Fibrosis (IPF), Chronic Obstructive Pulmonary Disorder (COPD), and asthma with ACE inhibitors, beta blockers or cortisone have undesired side effect profiles like physical dependency, increased heart rate, xerostomia, drowsiness or sedation.

An alternative approach of developing P2X3 inhibitors for the treatment of Refractory Chronic Cough like Afferent is doing with AF-219 (Abdulqawi et al, Lancet 2015, 385 (9974), 1198-1205), is accompanied by adverse effect on taste sensation.

Thus, there is an unmet need for medicaments which are effective in long-term and oral treatment of diseases which are associated with nerve fibre sensitization like Chronic Cough (CC), Idiopathic Pulmonary Fibrosis (IPF), Chronic Obstructive Pulmonary Disorder (COPD), and asthma, which do not have the disadvantages of the prior art as described above.

The underlying problem of the present invention therefore lies in the provision of medication for long-term oral treatment of diseases associated with nerve fibre sensitization like Chronic Cough (CC), Idiopathic Pulmonary Fibrosis (IPF), Chronic Obstructive Pulmonary Disorder (COPD), and asthma.

### SUMMARY OF THE INVENTION

It has now been found, and this constitutes the basis of the present invention, that compounds of the general formula (I)

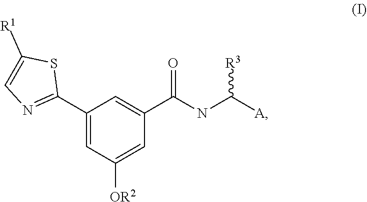

in which
R¹ represents a halogen atom, C₁-C₄-alkyl or C₃-C₆-cycloalkyl, wherein C₁-C₄-alkyl is optionally substituted with 1-5 halogen atoms which are the same or different;
R² represents —C₂-C₆-alkyl-OR⁴, —(CH₂)_{q}—(C₃-C₇-cycloalkyl), —(CH₂)_{q}-(6- to 12-membered heterobicycloalkyl), —(CH₂)_{q}-(4- to 7-membered heterocycloalkyl), —(CH₂)_{q}-(5- to 10-membered heteroaryl) or —C₂-C₆-alkynyl; and
   wherein said —(CH₂)_{q}—(C₃-C₇-cycloalkyl), —(CH₂)_{q}-(6- to 12-membered heterobicycloalkyl) and —(CH₂)_{q}-(4- to 7-membered heterocycloalkyl) are optionally substituted with one or more substituents which are the same or different, at any ring carbon atom and selected from the group consisting of
   C₁-C₄ alkyl, optionally substituted with 1-5 halogen atoms which are the same or different, a halogen atom, —NR^{a}R^{b}, COOR⁵ and oxo (═O); and
   wherein independently any ring nitrogen atom, if present in said —(CH₂)_{q}-(6- to 12-membered heterobicycloalkyl) and —(CH₂)_{q}-(4- to 7-membered heterocycloalkyl) is substituted with R^{c}; and
   wherein said —(CH₂)_{q}-(5- to 10-membered heteroaryl) is optionally substituted with one or more substituents which are the same or different, and selected from the group consisting of C₁-C₄-alkyl, optionally substituted with 1-5 halogen atoms which are the same or different, a halogen atom, —NR^{a}R^{b} and —COOR⁵;
R³ represents hydrogen or C₁-C₄-alkyl, which is optionally substituted with 1-5 halogen atoms which are the same or different;
R⁴ and R⁵ represent hydrogen or C₁-C₄-alkyl;
R^{a} and R^{b} represent hydrogen or C₁-C₄-alkyl;
R^{c} represents hydrogen, C₁-C₄-alkyl, optionally substituted with 1-5 halogen atoms which are the same or different, —C(O)—C₁-C₄-alkyl, or —C(O)—C₁-C₄-alkyl;
A represents 5- to 10-membered heteroaryl which is optionally substituted with one or more substituents, which are the same or different, and selected from the group consisting of a halogen atom, C₁-C₃-alkyl, and C₁-C₃-alkoxy, wherein C₁-C₃-alkyl and C₁-C₃-alkoxy are optionally substituted with 1-5 halogen atoms which are the same or different;
q represents an integer of 0, 1, or 2;

or an isomer, enantiomer, diastereomer, racemate, hydrate, solvate, or a salt thereof, or a mixture of same can be used for the treatment or prophylaxis of diseases or disorders which are associated with nerve fibre sensitization, in particular for the treatment of Chronic Cough (CC), Idiopathic Pulmonary Fibrosis (IPF), Chronic Obstructive Pulmonary Disorder (COPD), and asthma.

By providing said treatment options, it is possible to solve the problem of having significant side effects known from present therapies for Chronic Cough (CC), Idiopathic Pulmonary Fibrosis (IPF), Chronic Obstructive Pulmonary Disorder (COPD), and asthma.

The prevention of significant side effects to important physiological functions, i.e. taste, wakefulness, or heart rate, that may weaken the potential clinical effectiveness of medicaments, is an advantage of this invention.

That means for example the avoidance of negatively effecting important physiological functions, like taste sensation, the avoidance of physical dependency, increased heart rate, xerostomia, constipation, nausea, drowsiness or sedation, which all have severe impact to the quality of life of patients. This makes it possible to provide a treatment for Chronic Cough (CC), Idiopathic Pulmonary Fibrosis (IPF), Chronic Obstructive Pulmonary Disorder (COPD), and asthma, which is useful for long-term treatment of the mentioned diseases. Furthermore, the oral treatment of Chronic Cough (CC), Idiopathic Pulmonary Fibrosis (IPF), Chronic Obstructive Pulmonary Disorder (COPD), and asthma, is possible with the provided treatment approach.

The present invention is based on the discovery that compounds of general formula (I) are highly potent and sufficient selective at the P2X3 receptor. Therefore the subject matter of the present invention is directed to the use of compounds of general formula (I) or a pharmaceutically acceptable salt thereof for the treatment or prophylaxis of diseases or disorders, which are associated with nerve fibre sensitization.

### DESCRIPTION OF FIGURES

FIG. 1 shows the vagal depolarization and the inhibition of human vagus nerve by compounds of general formula (I), i.e. patent example 11 and 348 as described in WO2016/091776 in comparison with Afferent's AF-219.

### DETAILED DESCRIPTION OF THE INVENTION

The terms as mentioned in the present text have preferably the following meanings:

The term “halogen atom”, “halo-” or “Hal-” is to be understood as meaning a fluorine, chlorine, bromine or iodine atom, preferably a fluorine or a chlorine atom.

The term “alkyl” is to be understood as meaning a linear or branched, saturated, monovalent hydrocarbon group with the number of carbon atoms as specified and having as a rule, 2 to 6 in case of R², and 1 to 4 for all other alkyl substituents, preferably 1 to 3, carbon atoms, by way of example and by preference a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, n-propyl, n-butyl, iso-propyl, iso-butyl, sec-butyl, tert-butyl group, more particularly 1, 2 or 3 carbon atoms (“C₁-C₃-alkyl”), e.g. a methyl, ethyl, n-propyl- or iso-propyl group, and even more particularly 1 or 2 carbon atoms (“C₁-C₂-alkyl”), e.g. a methyl or ethyl group.

The term “C₁-C₄-alkyl, optionally substituted with 1-5 halogen atoms”, or in analogy “C₁-C₃-alkyl, optionally substituted with 1-5 halogen atoms” or “C₁-C₂-alkyl which are optionally substituted with 1-5 halogen atoms”, is to be understood as meaning a linear or branched, saturated, monovalent hydrocarbon group in which the term “C₁-C₄-alkyl”, “C₁-C₃-alkyl” or “C₁-C₂-alkyl” is defined supra, and in which one or more hydrogen atoms is replaced by a halogen atom, which are the same or different, i.e. one halogen atom being independent from another. In particular, halogen is fluorine or chlorine.

The term “C₁-C₄-alkyl, optionally substituted with 1-5 fluorine atoms”, or in analogy “C₁-C₃-alkyl, optionally substituted with 1-5 fluorine atoms” or “C₁-C₂-alkyl, optionally substituted with 1-5 fluorine atoms”, is to be understood as meaning a linear or branched, saturated, monovalent hydrocarbon group in which the term “C₁-C₄-alkyl”, “C₁-C₃-alkyl” or “C₁-C₂-alkyl” is defined supra, and in which one or more hydrogen atoms is replaced by a fluorine atom.

Said “C₁-C₄-alkyl, optionally substituted with 1-5 fluorine atoms” or “C₁-C₄-alkyl group, optionally substituted with 1-5 halogen atoms” is, for example, —CH₂CH₂CH₂CF₃.

Similarly, the above-mentioned applies to “C₁-C₃-alkyl, optionally substituted with 1-5 halogen atoms”, or “C₁-C₂-alkyl, optionally substituted with 1-5 halogen atoms”, or “C₁-C₃-alkyl, optionally substituted with 1-5 fluorine atoms”, or “C₁-C₂-alkyl, optionally substituted with 1-5 fluorine atoms”. Thus said “C₁-C₃-alkyl optionally substituted with 1-5 halogen atoms” or “C₁-C₃-alkyl optionally substituted with 1-5 fluorine atoms” is, for example, —CH₂CH₂CF₃.

Said “C₁-C₂-alkyl optionally substituted with 1-5 halogen atoms” or “C₁-C₂-alkyl optionally substituted with 1-5 fluorine atoms” is, for example, —CF₃, —CHF₂, —CH₂F, —CF₂CF₃, —CH₂CHF₂, or —CH₂CF₃.

Under the proviso, that R² in formula (I) or (a) is —C₂-C₆-alkyl-OR⁴, “C₂-C₆-alkyl” is to be understood as C₁-C₅-alkylene which is bound to the phenolic oxygen via —CH₂— group. For example C₁-C₅-alkylene is methylene, ethylene, propylene, butylene, pentylene, iso-propylene, iso-butylene, sec-butylene, tert-butylene, iso-pentylene, 2-methylbutylene, 1-methylbutylene, 1-ethylpropylene, 1,2-dimethylpropylene, neo-pentylene, 1,1-dimethylpropylene.

Under the proviso, that R² in formula (I) or (a) is —C₂-C₆-alkyl-OR⁴, “C₂-C₆-alkyl” is also to be understood as C₁-C₄-alkylene which is bound to the phenolic oxygen via —CH—CH₃ group.

Under the proviso, that R² in formula (I) or (a) is —C₂-C₄-alkyl-OR⁴, “C₂-C₄-alkyl” is to be understood as C₁-C₃-alkylene which is bound to the phenolic oxygen via —CH₂— group. Under the proviso that R² in formula (I) or (a) is —C₂-C₄-alkyl-OR⁴, “C₂-C₄-alkyl” is also to be understood as C₁-C₂-alkylene which is bound to the phenolic oxygen via —CH—CH₃ group.

Under the proviso, that R² in formula (I) or (a) is —C₂-C₄-alkyl-OH, “C₂-C₄-alkyl” is to be understood as C₁-C₃-alkylene which is bound to the phenolic oxygen via —CH₂— group. Under the proviso that R² in formula (I) or (a) is —C₂-C₄-alkyl-OH, “C₂-C₄-alkyl” is also to be understood as C₁-C₂-alkylene which is bound to the phenolic oxygen via —CH—CH₃ group.

Under the proviso, that R² in formula (I) or (a) is —C₂-C₆-alkyl-OR⁴, “—OR⁴” is either at a tertiary, secondary or primary carbon atom of the —C₂-C₆-alkyl chain.

Under the proviso, that R² in formula (I) or (a) is —C₂-C₄-alkyl-OR⁴, “—OR⁴” is either at a tertiary, secondary or primary carbon atom of the —C₂-C₄-alkyl chain.

Under the proviso, that R² in formula (I) or (a) is —C₂-C₄-alkyl-OH, “—OH” is either at a tertiary, secondary or primary carbon atom of the —C₂-C₄-alkyl chain.

For example, said —C₂-C₆-alkyl-OR⁴ is 3-hydroxybutan-2-yl, (2R,3R)-3-hydroxybutan-2-yl, (2S,3S)-3-hydroxybutan-2-yl, (2R,3S)-3-hydroxybutan-2-yl, (2S,3R)-3-hydroxybutan-2-yl, (2R,3R)-3-methoxybutan-2-yl, (2S,3S)-3-methoxybutan-2-yl, (2R,3S)-3-methoxybutan-2-yl, (2S,3R)-3-methoxybutan-2-yl, 3-methoxybutan-2-yl, 2-hydroxy-2-methylpropan-1-yl, 2-methoxy-2-methylpropan-1-yl, 3-hydroxypropan1-yl, 3-hydroxybutan-1-yl, 3-hydroxy-3-methylbutan-1-yl, 3-hydroxy-2-methylbutan-1-yl, 3-hydroxy-2,2-dimethylpropan-1-yl, 4-hydroxy-3-methylbutan-2-yl, 4-hydroxy-3-methylpent-1-yl, 4-hydroxy-4-methylpent-1-yl, 2-hydroxy-2-methylpropan-1-yl, 2-methoxy-2-methyl-propan-1-yl, 2-methoxyethan-1-yl, 3-methoxypropan-1-yl, 4-methoxybutan-1-yl, 2-ethoxyethan-1-yl, 3-ethoxypropan-1-yl, 4-ethoxybutan-1-yl, 2-iso-propoxyethan-1-yl, 3-iso-propoxypropan-1-yl, 4-iso-propoxybutan-1-yl, 2-hydroxyethan-1-yl, 3-hydroxy-propan-1-yl, 4-hydroxybutan-1-yl, preferably 3-hydroxybutan-2-yl, (2R,3R)-3-hydroxybutan-2-yl, (2S,3S)-3-hydroxybutan-2-yl, (2R,3S)-3-hydroxybutan-2-yl, (2S,3R)-3-hydroxybutan-2-yl, more preferably (2R,3R)-3-hydroxybutan-2-yl, (2S,3S)-3-hydroxybutan-2-yl.

For example, said —C₂-C₄-alkyl-OR⁴ or —C₂-C₄-alkyl-OH is preferably 3-hydroxybutan-2-yl, (2R,3R)-3-hydroxybutan-2-yl, (2S,3S)-3-hydroxybutan-2-yl, (2R,3S)-3-hydroxybutan-2-yl, (2S,3R)-3-hydroxybutan-2-yl, more preferably (2R,3R)-3-hydroxybutan-2-yl, (2S,3S)-3-hydroxybutan-2-yl.

The term “alkoxy” is to be understood as meaning a linear or branched, saturated, monovalent, hydrocarbon group of formula —O-alkyl, in which the term “alkyl” is defined as meaning a linear or branched, saturated, monovalent hydrocarbon group with the number of carbon atoms as specified and having as a rule, 1 to 3, preferably 1 to 2 alkyl substituents, especially preferably 1, carbon atoms. Particularly, said group has 1, 2 or 3 carbon atoms (“C₁-C₃-alkoxy”), e.g. a methoxy, ethoxy, n-propoxy or iso-propoxy group, and even more particularly 1 or 2 carbon atoms (“C₁-C₂-alkoxy”), e.g. a methoxy or ethoxy group.

The term “C₁-C₃-alkoxy optionally substituted with 1-5 halogen atoms” is to be understood as meaning a linear or branched, saturated, monovalent hydrocarbon group in which the term “C₁-C₃-alkoxy” is defined supra, and in which one or more hydrogen atoms is replaced by a halogen atom, which are the same or different, i.e. one halogen atom being independent from another. In particular, halogen is fluorine or chlorine.

Said “C₁-C₃-alkoxy” group is optionally substituted with 1 to 5 fluorine atoms, for example, —OCF₃, —OCHF₂, —OCH₂F, —OCF₂CF₃, —OCH₂CHF₂, —OCH₂CF₃, —OCH₂CH₂CF₃, or —OCH₂CF₂CF₃. In particular, said “C₁-C₃-alkoxy” group optionally substituted with fluorine is —OCF₃.

The term “C₂-C₆-alkynyl” is to be understood as meaning a linear or branched, monovalent hydrocarbon group which contains one or more triple bonds, preferably one triple bond, and which contains 2, 3, 4, 5 or 6 carbon atoms, particularly 3 or 4 carbon atoms (“C₃-C₄-alkynyl”). Said C₂-C₆-alkynyl group is, for example, ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent2-ynyl, 1-methyl-pent-2-ynyl, 4-methylpent-1-ynyl, 3-methylpent-1-ynyl, 2-ethylbut-3-ynyl, 1-ethyl-but-3-ynyl, 1-ethylbut-2-ynyl, 1-propylprop-2-ynyl, 1-isopropylprop-2-ynyl, 2,2-di-methylbut-3-ynyl, 1,1-dimethylbut-3-ynyl, 1,1-dimethylbut-2-ynyl, or 3,3-dimethyl-but-1-ynyl group. Particularly, said alkynyl group is prop-1-ynyl or prop-2-ynyl.

The term “cycloalkyl” is to be understood as meaning a saturated, monovalent, monocyclic hydrocarbon ring with the number of carbon atoms as specified and having as a rule, 3 to 7 or 3 to 6 ring carbon atoms, preferably 3 to 4 ring carbon atoms.

“C₃-C₇-cycloalkyl” is to be understood as meaning a saturated, monovalent, monocyclic hydrocarbon ring which contains 3, 4, 5, 6 or 7 carbon atoms. Said C₃-C₇-cycloalkyl group is for example a cyclopropyl, cyclobutyl, cyclopentyl, cyclohexyl or cycloheptyl ring. Each hydrogen of a cycloalkyl carbon may be replaced by a substituent as further specified. Particularly, said ring contains 3, 4, 5 or 6 carbon atoms (“C₃-C₆-cycloalkyl”), preferably 3 or 4 carbon atoms (“C₃-C₄-cycloalkyl”).

In case of R² in formula (I) or (Ia), said “C₃-C₇-cycloalkyl” in “(CH₂)_{q}—(C₃-C₇-cycloalkyl)” is, unless indicated otherwise, optionally substituted with one or more substituents which are the same or different, at any ring carbon atom and selected from the group consisting of C₁-C₄ alkyl, optionally substituted with 1-5 halogen atoms which are the same or different, a halogen atom, —NR^{a}R^{b}, COOR⁵ and oxo (═O). In case of R² in formula (I) or (Ia), said “C₃-C₄-cycloalkyl” as such or “C₃-C₄-cycloalkyl” in “CH₂—(C₃-C₄-cycloalkyl)” is, unless indicated otherwise, optionally substituted with one or more substituents which are the same or different, at any ring carbon atom and selected from a group consisting of C₁-C₄-alkyl, optionally substituted with 1-5 halogen atoms which are the same or different, a halogen atom, —NR^{a}R^{b}, —COOR⁵ and oxo (═O).

The term “heterocycloalkyl” is to be understood as meaning a saturated, monovalent, monocyclic hydrocarbon ring with the number of ring atoms as specified in which one, two or three ring atoms of the hydrocarbon ring is/are replaced by one, two or three heteroatoms or heteroatom-containing groups independently selected from O, S, S(═O), S(═O)₂, or N.

“4- to 7-membered heterocycloalkyl” is to be understood as meaning a saturated, monovalent, monocyclic “heterocycloalkyl” ring as defined supra which contains 4, 5, 6 or 7 ring atoms.

Similarly, “4- to 6-membered heterocycloalkyl” is to be understood as meaning a saturated, monovalent, monocyclic “heterocycloalkyl” ring as defined supra which contains 4, 5 or 6 ring atoms.

In case of R² in formula (I) or (Ia), said 4- to 7-membered heterocycloalkyl or 4- to 6-membered heterocycloalkyl is, unless indicated otherwise, optionally substituted with one or more substituents which are the same or different, at any ring carbon atom and selected from the group consisting of C₁-C₄ alkyl, optionally substituted with 1-5 halogen atoms which are the same or different, a halogen atom, —NR^{a}R^{b}, COOR⁵ and oxo (═O); and wherein independently any ring nitrogen atom, if present in said 4- to 7-membered or 4- to 6-membered heterocycloalkyl is substituted with R^{c}; it being possible for said 4- to 7-membered or 4- to 6-membered heterocycloalkyl group to be attached to the rest of the molecule via any one of the carbon atoms or, if present, a nitrogen atom. Accordingly, any ring nitrogen atom if present in said 4- to 7-membered or 4- to 6-membered heterocycloalkyl group is only substituted with R^{c}, if the designated atom's normal valency under the existing circumstances is not exceeded.

Particularly, said 4- to 7-membered heterocycloalkyl can contain 3, 4, 5 or 6 carbon atoms, and one or two of the above-mentioned heteroatoms or heteroatom-containing groups provided that the total number of ring atoms is not greater than 7, more particularly said heterocycloalkyl can contain 3, 4 or 5 carbon atoms, and one or two of the above-mentioned heteroatoms or heteroatom-containing groups provided that the total number of ring atoms is not greater than 6 (a “4- to 6-membered heterocycloalkyl”).

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, or a 6-membered ring, such as tetrahydropyranyl, piperidinyl, morpholinyl, dithianyl, thiomorpholinyl, piperazinyl, or a 7-membered ring, such as a diazepanyl ring, for example.

Particularly, without being limited thereto, said heterocycloalkyl can be in a more preferred embodiment (3R)-tetrahydrofuran-3-yl, (3S)-tetrahydrofuran-3-yl, 4-methylmorpholin-2-yl, (2R)-4-methylmorpholin-2-yl, (2S)-4-methylmorpholin-2-yl, 4-methylmorpholin-3-yl, (3R)-4-methylmorpholin-3-yl, or (3S)-4-methylmorpholin-3-yl, most preferred (2R)-4-methylmorpholin-2-yl.

The term “6- to 12-membered heterobicycloalkyl” is to be understood as meaning a saturated, monovalent bicyclic hydrocarbon radical in which the two rings share one or two common ring atoms, and wherein said bicyclic hydrocarbon radical contains 5, 6, 7, 8, 9 or 10 carbon atoms and one, two or three heteroatoms or heteroatom-containing groups independently selected from O, S, S(═O), S(═O)₂, or N, provided that the total number of ring atoms is not greater than 12. Said 6- to 12-membered heterobicycloalkyl is, unless indicated otherwise, optionally substituted with one or more substituents, which are the same or different, at any ring carbon atom and selected from the group consisting of C₁-C₄ alkyl, optionally substituted with 1-5 halogen atoms which are the same or different, a halogen atom, —NR^{a}R^{b}, COOR⁵ and oxo (═O); and wherein independently any ring nitrogen atom, if present in said 6- to 12-membered heterobicycloalkyl is substituted with R^{c}; it being possible for said 6- to 12-membered heterobicycloalkyl to be attached to the rest of the molecule via any one of the carbon atoms or, if present, a nitrogen atom. Accordingly, any ring nitrogen atom if present in said 6- to 12-membered heterobicycloalkyl is only substituted with Rc, if the designated atom's normal valency under the existing circumstances is not exceeded. Said 6- to 12-membered heterobicycoalkyl is, for example, azabicyclo[3.3.0]octyl, azabicyclo-[4.3.0]nonyl, diazabicyclo[4.3.0]nonyl, oxazabicyclo[4.3.0]nonyl, thiazabicyclo-[4.3.0]nonyl or azabicyclo[4.4.0]decyl.

Heterospirocycloalkyl and bridged heterocycloalkyl, as defined below, are also included within the scope of this definition.

The term “heterospirocycloalkyl” is to be understood as meaning a saturated, monovalent bicyclic hydrocarbon radical in which the two rings share one common ring atom, and wherein said bicyclic hydrocarbon radical contains 5, 6, 7, 8, 9 or 10 carbon atoms, and one, two or three heteroatoms or heteroatom-containing groups independently selected from O, S, S(═O), S(═O)₂, or N, provided that the total number of ring atoms is not greater than 12. It is possible for said heterospirocycloalkyl to be attached to the rest of the molecule via any one of the carbon atoms or, if present, a nitrogen atom. Said heterospirocycloalkyl is, for example, azaspiro[2.3]hexyl, azaspiro[3.3]heptyl, oxaazaspiro[3.3]heptyl, thiaaza-spiro[3.3]heptyl, oxaspiro[3.3]heptyl, oxazaspiro[5.3]nonyl, oxazaspiro[4.3]octyl, oxazaspiro[5.5]undecyl, diazaspiro[3.3]heptyl, thiazaspiro[3.3]heptyl, thiazaspiro-[4.3]octyl, or azaspiro[5.5]decyl.

The term “bridged heterocycloalkyl” is to be understood as meaning a saturated, monovalent bicyclic hydrocarbon radical in which the two rings share two common ring atoms which are not immediately adjacent, and wherein said bicyclic hydrocarbon radical contains 5, 6, 7, 8, 9 or 10 carbon atoms, and one, two or three heteroatoms or heteroatom-containing groups independently selected from O, S, S(═O), S(═O)₂, or N, provided that the total number of ring atoms is not greater than 12. It is possible for said bridged heterocycloalkyl to be attached to the rest of the molecule via any one of the carbon atoms or, if present, a nitrogen atom. Said bridged heterocycloalkyl is, for example, azabicyclo[2.2.1]heptyl, oxazabicyclo[2.2.1]heptyl, thiazabicyclo[2.2.1]heptyl, diazabicyclo[2.2.1]heptyl, azabicyclo[2.2.2]octyl, diazabicyclo[2.2.2]octyl, oxazabicyclo[2.2.2]octyl, thiazabicyclo[2.2.2]octyl, azabicyclo[3.2.1]octyl, diazabicyclo[3.2.1]octyl, oxazabicyclo-[3.2.1]octyl, thiazabicyclo[3.2.1]octyl, azabicyclo[3.3.1]nonyl, diazabicyclo[3.3.1]-nonyl, oxazabicyclo[3.3.1]nonyl, thiazabicyclo[3.3.1]nonyl, azabicyclo[4.2.1]nonyl, diazabicyclo[4.2.1]nonyl, oxazabicyclo[4.2.1]nonyl, thiazabicyclo[4.2.1]nonyl, azabicyclo[3.3.2]decyl, diazabicyclo[3.3.2]decyl, oxazabicyclo[3.3.2]decyl, thiazabicyclo[3.3.2]decyl, or azabicyclo[4.2.2]decyl.

The term “heteroaryl” is understood as meaning a monovalent, monocyclic or bicyclic hydrocarbon ring system with at least one aromatic ring with the number of ring system atoms as specified and wherein one, two or three ring atoms of the monovalent, monocyclic or bicyclic hydrocarbon ring system is/are replaced by one, two or three heteroatoms or heteroatom-containing groups independently selected from O, S, S(═O), S(═O)₂, or N.

“5- to 10-membered heteroaryl” is understood as meaning a heteroaryl having 5, 6, 7, 8, 9 or 10 ring atoms (a “5- to 10-membered heteroaryl”) and wherein one, two or three ring atoms of the monovalent, monocyclic or bicyclic hydrocarbon ring system is/are replaced by one, two or three heteroatoms or heteroatom-containing groups independently selected from O, S, S(═O), S(═O)₂, or N. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc. and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridinyl, pyridazinyl, pyrimidinyl, pyrazinyl, triazinyl, etc., and benzo derivatives thereof, such as, for example, quinolinyl, quinazolinyl, isoquinolinyl, etc.; indolizinyl, and benzo derivatives thereof; or cinnolinyl, phthalazinyl, quinazolinyl, quinoxalinyl, etc.

In case of R² of formula (I) or (Ia), said 5- to 10-membered heteroaryl is, unless indicated otherwise, optionally substituted with one or more substituents which are the same or different, and selected from the group consisting of C₁-C₄-alkyl, optionally substituted with 1-5 halogen atoms which are the same or different, a halogen atom, —NR^{a}R^{b} and —COOR⁵.

In case of R² of formula (I) or (Ia), said 5- to 10-membered heteroaryl optionally substituted as described above, can be in particular substituted with C₁-C₂-alkyl at any ring N, if present.

In case of A of formula (I) or (Ia), said 5- to 10-membered heteroaryl is, unless indicated otherwise, optionally substituted with one or more substituents, which are the same or different, and selected from the group consisting of a halogen atom, C₁-C₃-alkyl, and C₁-C₃-alkoxy, wherein C₁-C₃-alkyl and C₁-C₃-alkoxy are optionally substituted with 1-5 halogen atoms which are the same or different.

In case of A of formula (I) or (Ia), a “5- or 6-membered heteroaryl” is understood as meaning a heteroaryl having 5 or 6 ring atoms and wherein one, two or three ring atoms of the hydrocarbon ring system is/are replaced by one, two or three heteroatoms or heteroatom-containing groups independently selected from O, S, S(═O), S(═O)₂, or N. Said “5- or 6-membered heteroaryl” is, unless indicated otherwise, optionally substituted with one or more substituents, which are the same or different, and selected from the group consisting of a halogen atom, C₁-C₃-alkyl, and C₁-C₃-alkoxy, wherein C₁-C₃-alkyl and C₁-C₃ alkoxy are optionally substituted with 1-5 halogen atoms which are the same or different

A 5-membered heteroaryl group is preferably selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl.

A 6-membered heteroaryl group is preferably selected from pyridinyl, pyridazinyl, pyrimidinyl, pyrazinyl, triazinyl.

In particular, said 5- or 6-membered heteroaryl is, optionally substituted with preferably one or two substituents, which are the same or different, and selected from a fluorine or chlorine atom, C₁-C₂-alkyl, optionally substituted with 1-5 fluorine atoms, or C₁-C₂-alkoxy, optionally substituted with 1-5 fluorine atoms.

In particular, said 5- or 6-membered heteroaryl is a 6-membered heteroaryl with one or two nitrogen atom(s) and is optionally substituted with one or two substituents, which are the same or different, and selected from a fluorine or chlorine atom, C₁-C₂-alkyl, optionally substituted with 1-5 fluorine atoms, or C₁-C₂-alkoxy, optionally substituted with 1-5 fluorine atoms.

Preferably said 6-membered heteroaryl is CF₃-pyrimidinyl, most preferably 2-CF₃-pyrimidin-5-yl. Also preferred is CF₃-pyridazinyl, most preferably 6-CF₃-pyridazin-3-yl.

In general, and unless otherwise mentioned, the term “heteroaryl” includes all possible isomeric forms thereof, e.g. the positional isomers thereof. Thus, for some illustrative non-restricting example, the term pyridyl includes pyridin-2-yl, pyridin-3-yl, and pyridin-4-yl; or the term pyrimidinyl includes pyrimidin-2-yl, pyrimidin-4-yl and pyrimidin-5-yl; or the term pyridazinyl includes pyridazin-3-yl and pyridazin-4-yl; or the term thiazolyl includes 1,3-thiazol-5-yl, 1,3-thiazol-4-yl and 1,3-thiazol-2-yl.

The term “C₁-C₄” as used throughout this text is to be understood as meaning a group having a finite number of carbon atoms of 1 to 4, i.e. 1, 2, 3 or 4 carbon atoms, e.g. in the context of the definition of “C₁-C₄-alkyl”, it is to be understood as meaning an alkyl group having a finite number of carbon atoms of 1 to 4, i.e. 1, 2, 3 or 4 carbon atoms.

The term “C₂-C₆” as used throughout this text is to be understood as meaning a group having a finite number of carbon atoms of 2 to 6, i.e. 2, 3, 4, 5 or 6 carbon atoms, e.g. in the context of the definition of “C₂-C₆-alkyl”, it is to be understood as meaning an alkyl group having a finite number of carbon atoms of 2 to 6, i.e. 2, 3, 4, 5 or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

The term “C₁-C₃” as used in the context of the definition “C₁-C₃-alkoxy” is to be understood as meaning an alkoxy group, having a finite number of carbon atoms of 1 to 3, i.e. 1, 2 or 3 carbon atoms.

The same applies to other mentioned “alkyl”, alkynyl or “alkoxy” as mentioned herein and as it is to be understood by a skilled person.

It is to be understood further that for example a term “C₁-C₆” is to be interpreted as any sub-range comprised therein, e.g. C₁-C₆, C₂-C₃, C₂-C₆, C₃-C₄, C₁-C₂, C₁-C₃, C₁-C₄, C₁-C₅; particularly C₁-C₂, C₁-C₃, C₁-C₄, C₁-C₅, C₁-C₆; more particularly C₁-C₄.

Similarly, the mentioned above applies to “C₁-C₄-alkyl”, “C₁-C₃-alkyl”, “C₁-C₃-alkoxy”, “C₁-C₂-alkyl” or “C₁-C₂-alkoxy” optionally substituted with 1-5 halogen which are the same or different.

Similarly, as used herein, the term “C₂-C₆”, as used throughout this text, e.g. in the context of the definitions of “C₂-C₆-alkynyl”, is to be understood as meaning an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃ and C₂-C₄.

Further, as used herein, the term “C₃-C₆”, as used throughout this text, is to be understood as meaning a group having a finite number of carbon atoms of 3 to 7, i.e. 3, 4, 5, 6 or 7 carbon atoms, e.g. in the context of the definition of “C₃-C₇-cycloalkyl”, it is to be understood as meaning a cycloalkyl group having a finite number of carbon atoms of 3 to 7, i.e. 3, 4, 5, 6 or 7 carbon atoms. It is to be understood further that said term “C₃-C₇” is to be interpreted as any sub-range comprised therein, e.g. C₃-C₆, C₄-C₅, C₃-C₅, C₃-C₄, C₄-C₆, C₅-C₇; particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) ranges from 1 to 5, in particular from 1 to 3.

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, sulphur, fluorine and chlorine such as ²H (deuterium), ³H (tritium), ¹¹C, ¹³C, ¹⁴C, ¹⁵N, ¹⁷O, ¹⁸O, ³³S, ³⁴S, ³⁵S, ³⁶S, ¹⁸F and ³⁶Cl, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful.

The optically active forms of compounds of formula (I) can likewise be obtained by chiral syntheses utilizing optically active starting materials.

In order to limit different types of isomers from each other reference is made to IUPAC Rules Section E (Pure Appl Chem 45, 11-30, 1976).

Further, compounds may exist as tautomers.

A compound of general formula (I) includes all possible tautomers as single tautomers, or as any mixture of said tautomers, in any ratio.

The present invention also relates to the use of useful forms of compounds of formula (I), such as metabolites, hydrates, solvates, prodrugs, salts, in particular pharmaceutically acceptable salts, and co-precipitates.

Where the plural form of the word compounds, salts, polymorphs, hydrates, solvates and the like, is used herein, this is taken to mean also a single compound, salt, polymorph, isomer, hydrate, solvate or the like.

By “stable compound’ or “stable structure” is meant a compound that is sufficiently robust to survive isolation to a useful degree of purity from a reaction mixture, and formulation into an efficacious therapeutic agent.

Compounds of formula (I) can exist as a hydrate, or as a solvate, wherein compounds of formula (I) contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. A compound of general formula (I) include all such hydrates or solvates.

Further, compounds of formula (I) can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of compounds of formula (I). For example, see S. M. Berge, et al. “Pharmaceutical Salts,” J. Pharm. Sci. 1977, 66, 1-19. A suitable pharmaceutically acceptable salt of the compounds of the present invention may be, for example, an acid-addition salt of compounds of formula (I) bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of formula (I) which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and stearyl chlorides, bromides and iodides, aralkyl halides like benzyl and phenethyl bromides and others.

Those skilled in the art will further recognise that acid addition salts of compounds of formula (I) may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of compounds of formula (I) as single salts, or as any mixture of said salts, in any ratio.

Unless otherwise indicated, compounds of formula (I) are also referred to isomers, enantiomers, diastereomers, racemates, hydrates, solvates, or a salt thereof, or a mixture of same.

As used herein, the term “in vivo hydrolysable ester” is understood as meaning an in vivo hydrolysable ester of a compound of formula (I) containing a carboxy or hydroxy group, for example, a pharmaceutically acceptable ester which is hydrolysed in the human or animal body to produce the parent acid or alcohol.

Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds of formula (I). An in vivo hydrolysable ester of a compound of formula (I) containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of compounds of formula (I), either as single polymorphs, or as a mixture of more than one polymorph, in any ratio.

According to the present invention, the term Chronic Cough (CC) is understood as chronic cough lasting longer than 8 weeks, and describing a cough disease of patient populations suffering from Idiopathic Chronic Cough (ICC), synonymously known as Unexplained Chronic Cough (UCC), or Refractory Chronic Cough (RCC). Said Chronic Cough (CC) is also called Refractory or Unexplained Chronic Cough (RUCC).

According to the present invention, the term Chronic Cough (CC) is understood as chronic cough lasting longer than 8 weeks, when no cause could be identified, or Refractory Chronic Cough RCC. Said Chronic Cough (CC) is also called Refractory or Unexplained Chronic Cough (RUCC).

According to the present invention, the abbreviation RUCC is used for a chronic cough disease as defined above as Chronic Cough (CC), also called Refractory or Unexplained Chronic Cough according to the definition above (ICC also known as UCC), which is a chronic cough disease of patient populations suffering from ICC, synonymously known as UCC, or RCC.

Refractory Chronic Cough refers to chronic cough, which might be persistent after diagnosis and treatment of cough related conditions (such as gastroesophageal reflux disease, asthma (RCC).

Contrary to the above-mentioned, the term Refractory and Unexplained Chronic Cough as used herein is referred to a cough diseases in patients suffering from a chronic cough lasting longer than 8 weeks, when no cause could be identified, i.e. ICC also known as UCC, and when said cough persists after therapy with drugs commonly used for the treatment of cough (empiric therapy). This is therefore considered a subgroup of ICC also known as UCC. Said type of cough disease is described in the course of the present invention without using any abbreviation.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route, and form of administration, the judgment of the attending medical or veterinary practitioner, and other factors.

“Treating” or “treatment” of a disease state includes: (i) inhibiting the disease state, i.e. arresting the development of the disease state or its clinical symptoms, or (ii) relieving the disease state, i.e. causing temporary or permanent regression of the disease state or its clinical symptoms.

“Preventing” or “prevention” of a disease state includes causing the clinical symptoms of the disease state not to develop in a subject that may be exposed to or predisposed to the disease state, but does not yet experience or display symptoms of the disease state. For example, treating or preventing a respiratory disease or disorder includes treating or preventing the symptoms the disorder such as cough and/or urge to cough associated with a respiratory disease.

“Method of treatment” or “use of a compound of general formula (I)” or “compound of general formula (I) for the use in the treatment or prophylaxis” includes treatment of Chronic Cough (CC), Idiopathic Pulmonary Fibrosis (IPF), Chronic Obstructive Pulmonary Disorder (COPD), and asthma with compounds of general formula (I). Furthermore, it includes a long-term treatment or prophylaxis of Chronic Cough (CC), Idiopathic Pulmonary Fibrosis (IPF), Chronic Obstructive Pulmonary Disorder (COPD), and asthma with compound of general formula (I). An oral treatment or prophylaxis of Chronic Cough (CC), Idiopathic Pulmonary Fibrosis (IPF), Chronic Obstructive Pulmonary Disorder (COPD), and asthma with a compound of general formula (I) is included as well. Furthermore, it includes a long-term and oral treatment or prophylaxis of Chronic (CC), Idiopathic Pulmonary Fibrosis (IPF), Chronic Obstructive Pulmonary Disorder (COPD), and asthma with a compound of general formula (I).

In accordance with a first aspect, the present invention covers the use of compounds of general formula (I) for the treatment or prophylaxis of Chronic Cough (CC), Idiopathic Pulmonary Fibrosis (IPF), Chronic Obstructive Pulmonary Disorder (COPD), and asthma.

In accordance with a further aspect, the present invention relates to the use of compounds of general formula (I) for the treatment and prophylaxis of Chronic Cough (CC).

In accordance with a further aspect, the present invention relates to the use of compounds of general formula (I) for the treatment and prophylaxis of Refractory or Unexplained Chronic Cough (RUCC).

In accordance with a further aspect, the present invention relates to the use of compounds of general formula (I) for the treatment or prophylaxis of Idiopathic Chronic Cough (ICC) also known as Unexplained Chronic Cough (UCC).

In accordance with a further aspect, the present invention relates to the use of compounds of general formula (I) for the treatment or prophylaxis of Refractory Chronic Cough (RCC).

In accordance with a second aspect, the present invention relates to the use of compounds of general formula (I) for long-term treatment of Chronic Cough (CC), Idiopathic Pulmonary Fibrosis (IPF), Chronic Obstructive Pulmonary Disorder (COPD), and asthma.

In accordance with a third aspect, the present invention relates to the use of compounds of general formula (I) for the oral treatment or oral prophylaxis of Chronic Cough (CC), Idiopathic Pulmonary Fibrosis (IPF), Chronic Obstructive Pulmonary Disorder (COPD), and asthma.

In accordance with a fourth aspect, the present invention relates to the use of compounds of general formula (I) for long-term and oral treatment of Chronic Cough (CC), Idiopathic Pulmonary Fibrosis (IPF), Chronic Obstructive Pulmonary Disorder (COPD), and asthma.

In accordance with a fifth aspect, the present invention relates to the use of compounds of general formula (I) for long-term and oral treatment of Chronic Cough (CC).

In accordance with a further aspect, the present invention relates to the use of compounds of general formula (I) for long-term and oral treatment of Refractory or Unexplained Chronic Cough (RUCC).

In accordance with a sixth aspect, the present invention relates to the use of compounds of general formula (I) for long-term and oral treatment of Idiopathic Chronic Cough (ICC) also known as Unexplained Chronic Cough (UCC).

In accordance with a further aspect, the present invention relates to the use of compounds of general formula (I) for long-term and oral treatment of Refractory Chronic Cough (RCC).

In accordance with a seventh aspect, the present invention covers a method of treatment or prophylaxis of Chronic Cough (CC), Idiopathic Pulmonary Fibrosis (IPF), Chronic Obstructive Pulmonary Disorder (COPD), and asthma in a subject in need thereof.

In accordance with a further aspect, the present invention covers a method of treatment or prophylaxis of Chronic Cough (CC) in a subject in need thereof.

In accordance with a further aspect, the present invention covers a method of treatment or prophylaxis of Refractory or Unexplained Chronic Cough (RUCC) in a subject in need thereof.

In accordance with a further aspect, the present invention covers a method of treatment or prophylaxis of Idiopathic Chronic Cough (ICC) also known as Unexplained Chronic Cough (UCC) in a subject in need thereof.

In accordance with a further aspect, the present invention covers a method of treatment or prophylaxis of Refractory Chronic Cough (RCC) in a subject in need thereof.

In accordance with an eight aspect, the present invention relates to a method of long-term treatment of Chronic Cough (CC), Idiopathic Pulmonary Fibrosis (IPF), Chronic Obstructive Pulmonary Disorder (COPD), and asthma in a subject in need thereof.

In accordance with a further aspect, the present invention relates to a method of long-term treatment of Chronic Cough (CC) in a subject in need thereof.

In accordance with a further aspect, the present invention relates to a method of long-term treatment of Refractory or Unexplained Chronic Cough (RUCC) in a subject in need thereof.

In accordance with a further aspect, the present invention relates to a method of long-term treatment of Idiopathic Chronic Cough (ICC) also known as Unexplained Chronic Cough (UCC) in a subject in need thereof.

In accordance with a further aspect, the present invention relates to a method of long-term treatment of Refractory Chronic Cough (RCC) in a subject in need thereof.

In accordance with a ninth aspect, the present invention relates to a method of oral treatment or oral prophylaxis of Chronic Cough (CC), Idiopathic Pulmonary Fibrosis (IPF), Chronic Obstructive Pulmonary Disorder (COPD), and asthma in a subject in need thereof.

In accordance with a further aspect, the present invention relates to a method of oral treatment or oral prophylaxis of Chronic Cough (CC) in a subject in need thereof.

In accordance with a further aspect, the present invention relates to a method of oral treatment or oral prophylaxis of Refractory or Unexplained Chronic Cough (RUCC) in a subject in need thereof.

In accordance with a further aspect, the present invention relates to a method of oral treatment or oral prophylaxis of Idiopathic Chronic Cough (ICC) also known as Unexplained Chronic Cough (UCC) in a subject in need thereof.

In accordance with a further aspect, the present invention relates to a method of oral treatment or oral prophylaxis of Refractory Chronic Cough (RCC) in a subject in need thereof.

In accordance with a tenth aspect, the present invention relates to a method of long-term and oral treatment of Chronic Cough (CC), Idiopathic Pulmonary Fibrosis (IPF), Chronic Obstructive Pulmonary Disorder (COPD), and asthma in a subject in need thereof in a subject in need thereof.

In accordance with a eleventh aspect, the present invention relates to a method of long-term and oral treatment of Chronic Cough (CC) in a subject in need thereof.

In accordance with a further aspect, the present invention relates to a method of long-term and oral treatment of Refractory or Unexplained Chronic Cough (RUCC) in a subject in need thereof.

In accordance with a further aspect, the present invention relates to a method of long-term and oral treatment of Idiopathic Chronic Cough (ICC) also known as Unexplained Chronic Cough (UCC) in a subject in need thereof.

In accordance with a further aspect, the present invention relates to a method of long-term and oral treatment of Refractory Chronic Cough (RCC) in a subject in need thereof.

A method in accordance with the invention comprises administering to the subject in need thereof an effective amount of a compound of formula (I) or a pharmaceutically acceptable salt thereof. The method comprises administering an effective amount of a compound of formula (I).

A preferred embodiment of the invention relates to a method of long-term and oral treatment or prophylaxis of Refractory or Unexplained Chronic Cough (RUCC) in a subject in need thereof administering an effective amount of a compound of formula (I) or a pharmaceutically acceptable salt thereof.

Another preferred embodiment of the invention relates to the use of a compound of general formula (I) or a pharmaceutically acceptable salt thereof in the long-term and oral treatment or prophylaxis of Refractory or Unexplained Chronic Cough (RUCC).

Another preferred embodiment of the invention relates to a compound of general formula (I) or a pharmaceutically acceptable salt thereof for the use in the long-term and oral treatment or prophylaxis of Refractory or Unexplained Chronic Cough (RUCC).

Another preferred embodiment of the invention relates to a method of long-term and oral treatment or prophylaxis of a chronic cough disease defined by terms selected from the group consisting of Idiopathic Chronic Cough (ICC), Unexplained Chronic Cough (UCC), and Refractory Chronic Cough (RCC) with a compound of general formula (I) or a pharmaceutically acceptable salt thereof.

Another preferred embodiment of the invention relates to the use of a compound of general formula (I) or a pharmaceutically acceptable salt thereof in the long-term and oral treatment or prophylaxis of a chronic cough disease defined by terms selected from the group consisting of Idiopathic Chronic Cough (ICC), Unexplained Chronic Cough (UCC), and Refractory Chronic Cough (RCC).

Another preferred embodiment of the invention relates to a compound of general formula (I) or a pharmaceutically acceptable salt thereof for use in the long-term and oral treatment or prophylaxis of Refractory or Unexplained Chronic Cough (RUCC).

The present invention further relates to the use of pharmaceutical compositions and combinations comprising the compounds of general formula (I) for the treatment or prophylaxis of diseases or disorders which are associated with nerve fibre sensitization, in particular for the treatment of Chronic Cough, Idiopathic Pulmonary Fibrosis (IPF), Chronic Obstructive Pulmonary Disorder (COPD), and asthma.

The present invention further relates to the use of compounds of general formula (Ia),

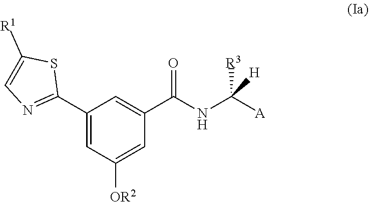

in which A, R¹, R² and R³ have the meanings as defined in formula (I), preferably R³ represents C₁-C₄-alkyl, more preferably methyl;
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment of Chronic Cough, Idiopathic Pulmonary Fibrosis (IPF), Chronic Obstructive Pulmonary Disorder (COPD), and asthma.

Another embodiment of the present invention relates to a method for using compounds of general formula (I), wherein
A represents an optionally substituted 5- or 6-membered heteroaryl, preferably an optionally substituted 6-membered heteroaryl; and

in which R¹, R² and R³ have the same meaning as defined in general formula (I), or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents an optionally substituted 5- or 6-membered heteroaryl, preferably an optionally substituted 6-membered heteroaryl; and

in which R¹, R² and R³ have the same meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Additionally, an embodiment of the present invention relates to a method for using compounds of general formula (I), wherein
R¹ represents C₁-C₄-alkyl, preferably methyl or ethyl; and

in which A, R² and R³ have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using are compounds of general formula (Ia), wherein
R¹ represents C₁-C₄-alkyl, preferably methyl or ethyl; and

in which A, R² and R³ have the same meaning as defined in general formula (Ia),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
R¹ represents a halogen atom, preferably chloro; and

in which A, R² and R³ have the same meaning as defined in general formula (I), or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
R¹ represents a halogen atom, preferably chloro; and

in which A, R² and R³ have the same meaning as defined in general formula (Ia), or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably compounds of general formula (Ia), wherein
R³ represents C₁-C₄-alkyl, preferably methyl; and

in which R¹, R² and A have the same meaning as defined in general formula (I), or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
R² represents —C₂-C₄-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl; and
   wherein said —CH₂—(C₃-C₄-cycloalkyl), C₃-C₄-cycloalkyl and —(CH₂)_{q}-(4- to 6-membered heterocycloalkyl) are optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c};
q represents an integer of 0; and

in which A, R^{c}, R¹ and R³ have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
R² represents —C₂-C₃-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl; and
   wherein said —CH₂—(C₃-C₄-cycloalkyl), C₃-C₄-cycloalkyl and —(CH₂)_{q}-(4- to 6-membered heterocycloalkyl) are optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c};
q represents an integer of 0; and

in which A, R^{c}, R¹ and R³ have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
R² represents —C₂-C₄-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl; and
   wherein said —CH₂—(C₃-C₄-cycloalkyl), C₃-C₄-cycloalkyl and —(CH₂)_{q}-(4- to 6-membered heterocycloalkyl) are optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c};
q represents an integer of 0; and

in which A, R^{c}, R¹ and R³ have the same meaning as defined in general formula (Ia),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
R² represents —C₂-C₃-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl; and
   wherein said —CH₂—(C₃-C₄-cycloalkyl), C₃-C₄-cycloalkyl and —(CH₂)_{q}-(4- to 6-membered heterocycloalkyl) are optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c};
q represents an integer of 0; and

in which A, R^{c}, R¹ and R³ have the same meaning as defined in general formula (a),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
R² represents —(CH₂)_{q}-(4- to 6-membered heterocycloalkyl); and wherein (CH₂)_{q}-(4- to 6-membered heterocycloalkyl) is optionally substituted with one or more substituents which are the same or different, at any ring carbon atom; and
   wherein independently any ring nitrogen atom, if present in said —(CH₂)_{q}-(4 to 6-membered heterocycloalkyl) is substituted with R^{c}; and wherein —(CH₂)_{q}-(4- to 6-membered heterocycloalkyl) is preferably —(CH₂)_{q}-morpholinyl; and
q represents an integer of 1; and

in which A, R^{c}, R¹ and R³ have the same meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
R² represents-(CH₂)_{q}-morpholinyl, wherein the ring nitrogen atom is substituted with R^{c}; and
R^{c} represents methyl;
q represents an integer of 1; and

in which A, R¹ and R³ have the same meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
R² represents —(CH₂)_{q}-(4- to 6-membered heterocycloalkyl); and wherein (CH₂)_{q}-(4- to 6-membered heterocycloalkyl) is optionally substituted with one or more substituents which are the same or different, at any ring carbon atom; and
   wherein independently any ring nitrogen atom, if present in said —(CH₂)_{q}-(4 to 6-membered heterocycloalkyl) is substituted with R^{c}; and wherein —(CH₂)_{q}-(4- to 6-membered heterocycloalkyl) is preferably —(CH₂)_{q}-morpholinyl; and
q represents an integer of 1; and

in which A, R^{c}, R¹ and R³ have the same meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
R² represents-(CH₂)_{q}-morpholinyl, wherein the ring nitrogen atom is substituted with R^{c}; and
R^{c} represents methyl;
q represents an integer of 1; and

in which A, R¹ and R³ have the same meaning as defined in general formula (a),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
R² represents —C₂-C₄-alkyl-OH; and

in which A, R¹ and R³ have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using to compounds of general formula (Ia), wherein
R² represents —C₂-C₄-alkyl-OH; and

in which A, R¹ and R³ have the same meaning as defined in general formula (Ia),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents an optionally substituted 5- or 6-membered heteroaryl, preferably an optionally substituted 6-membered heteroaryl; and
R¹ represents C₁-C₄-alkyl, preferably methyl or ethyl; and

in which R² and R³ have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents an optionally substituted 5- or 6-membered heteroaryl, preferably an optionally substituted 6-membered heteroaryl; and
R¹ represents C₁-C₄-alkyl, preferably methyl or ethyl; and

in which R² and R³ have the same meaning as defined in general formula (Ia),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents an optionally substituted 5- or 6-membered heteroaryl, preferably an optionally substituted 6-membered heteroaryl; and
R¹ represents a halogen atom, preferably chloro; and

in which R² and R³ have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents an optionally substituted 5- or 6-membered heteroaryl, preferably an optionally substituted 6-membered heteroaryl; and
R¹ represents a halogen atom, preferably chloro; and

in which R² and R³ have the same meaning as defined in general formula (Ia),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl; and
R³ represents C₁-C₄-alkyl, preferably methyl; and

in which R¹ and R² have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents C₁-C₄-alkyl, preferably methyl or ethyl; and
R³ represents C₁-C₄-alkyl, preferably methyl; and

in which R² has the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents a halogen atom, preferably chloro; and
R³ represents C₁-C₄-alkyl, preferably methyl; and

in which R² has the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents C₁-C₄-alkyl, preferably methyl or ethyl;
R² represents —C₂-C₄-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl; and
   wherein said —CH₂—(C₃-C₄-cycloalkyl), C₃-C₄-cycloalkyl and —(CH₂)_{q}-(4- to 6-membered heterocycloalkyl) are optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c};
R³ represents C₁-C₄-alkyl, preferably methyl; and
q represents an integer of 0,

wherein R^{c} is defined as in formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents C₁-C₄-alkyl, preferably methyl or ethyl;
R² represents —C₂-C₃-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl; and
   wherein said —CH₂—(C₃-C₄-cycloalkyl), C₃-C₄-cycloalkyl and —(CH₂)_{q}-(4- to 6-membered heterocycloalkyl) are optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c}; and
R³ represents C₁-C₄-alkyl, preferably methyl; and
q represents an integer of 0,

wherein R^{c} is defined as in formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents C₁-C₄-alkyl, preferably methyl or ethyl;
R² represents —(CH₂)_{q}-(4- to 6-membered heterocycloalkyl); and wherein (CH₂)_{q}-(4- to 6-membered heterocycloalkyl) is optionally substituted with one or more substituents which are the same or different, at any ring carbon atom; and
   wherein independently any ring nitrogen atom, if present in said —(CH₂)_{q}-(4 to 6-membered heterocycloalkyl) is substituted with R^{c}; wherein —(CH₂)_{q}-(4- to 6-membered heterocycloalkyl) is preferably —(CH₂)_{q}-morpholinyl;
R³ represents C₁-C₄-alkyl, preferably methyl; and
q represents an integer of 1;

wherein R^{c} is defined as in formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents C₁-C₄-alkyl, preferably methyl or ethyl;
R² represents —(CH₂)_{q}-morpholinyl, wherein the ring nitrogen atom is substituted with R^{c}; and
R^{c} represents methyl;
R³ represents C₁-C₄-alkyl, preferably methyl; and
q represents an integer of 1;
   or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents a halogen atom, preferably chloro;
R² represents —C₂-C₄-alkyl-OH, preferably 3-hydroxybutan-2-yl;
R³ represents C₁-C₄-alkyl, preferably methyl;
   or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably compounds of general formula (Ia), wherein
A represents 5- or 6-membered heteroaryl at least containing one or two nitrogen atom(s), preferably a 6-membered heteroaryl with one or two nitrogen atom(s),
   wherein said 5- or 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R¹ represents methyl or ethyl;
R² represents —C₂-C₃-alkyl-OR⁴, unsubstituted —CH₂—(C₃-C₄-cycloalkyl), unsubstituted C₃-C₄-cycloalkyl, unsubstituted (CH₂)_{q}-(4- to 6-membered heterocycloalkyl), or —C₂-C₄-alkynyl;
R³ represents methyl; and
q represents an integer of 0,
   or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment of and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably compounds of general formula (Ia), wherein
A represents 5- or 6-membered heteroaryl at least containing one or two nitrogen atom(s), preferably a 6-membered heteroaryl with one or two nitrogen atom(s),
   wherein said 5- or 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R¹ represents methyl or ethyl;
R² represents optionally substituted (CH₂)_{q}-(4- to 6-membered heterocycloalkyl), wherein —(CH₂)_{q}-(4- to 6-membered heterocycloalkyl) is optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c}; wherein —(CH₂)_{q}-(4- to 6-membered heterocycloalkyl) is preferably —(CH₂)_{q}-morpholinyl;
R³ represents methyl; and
q represents an integer of 1,

wherein R^{c} is defined as in formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably compounds of general formula (Ia), wherein
A represents 5- or 6-membered heteroaryl at least containing one or two nitrogen atom(s), preferably a 6-membered heteroaryl with one or two nitrogen atom(s),
   wherein said 5- or 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R¹ represents methyl or ethyl;
R² represents —(CH₂)_{q}-morpholinyl, wherein the ring nitrogen atom is substituted with R^{c} as defined in formula (I), preferably substituted with methyl;
R³ represents methyl; and
q represents an integer of 1,
   or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably compounds of general formula (Ia), wherein
A represents 5- or 6-membered heteroaryl at least containing one or two nitrogen atom(s), preferably a 6-membered heteroaryl with one or two nitrogen atom(s),
   wherein said 5- or 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R¹ represents a chloro atom;
R² represents —C₂-C₄-alkyl-OH, preferably 3-hydroxybutan-2-yl; and
R³ represents methyl,
   or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents pyrimidinyl, pyridazinyl, pyridinyl, pyrazinyl, thiazolyl or thiadiazolyl, preferably pyrimidinyl, pyridazinyl, thiazolyl or thiadiazolyl, more preferably pyrimidinyl, pyridazinyl or thiadiazolyl, wherein said pyrimidinyl, pyridazinyl, pyridinyl, pyrazinyl, thiazolyl and thiadiazolyl are optionally substituted; and

in which R¹, R² and R³ have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

In another preferred embodiment, the invention relates to use of compounds of general formula (Ia), wherein
A represents pyrimidinyl, pyridazinyl, pyridinyl, pyrazinyl, thiazolyl or thiadiazolyl, preferably pyrimidinyl, pyridazinyl, thiazolyl or thiadiazolyl, more preferably pyrimidinyl, pyridazinyl or thiadiazolyl, wherein said pyrimidinyl, pyridazinyl, pyridinyl, pyrazinyl, thiazolyl and thiadiazolyl are optionally substituted; and

in which R¹, R² and R³ have the same meaning as defined in general formula (Ia),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents CF₃-pyrimidinyl, preferably 2-CF₃-pyrimidin-5-yl; and

in which R¹, R² and R³ have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

In another preferred embodiment, the invention relates to compounds of general formula (Ia), wherein
A represents CF₃-pyrimidinyl, preferably 2-CF₃-pyrimidin-5-yl; and

in which R¹, R² and R³ have the same meaning as defined in general formula (Ia),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents CF₃-pyridazinyl, preferably 6-CF₃-pyridazin-3-yl; and

in which R¹, R² and R³ have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents CF₃-pyridazinyl, preferably 6-CF₃-pyridazin-3-yl; and

in which R¹, R² and R³ have the same meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using to compounds of general formula (I), wherein
R² represents cyclopropylmethyl, tetrahydrofuran-3-yl, tetrahydrofuran-2-ylmethyl, tetrahydrofuran-3-ylmethyl, prop-2-yn-1-yl, but-2-yn-1-yl, oxetan-3-yl, tetrahydropyran-4-yl, tetrahydro-2H-pyran-4-ylmethyl, pyridin-4-yl, pyridin-3-yl, 1,3,4-thiadiazol-2-yl, 1,3-thiazol-2-yl, 2,2-dimethyl-2-methoxyethyl, methoxyethyl, piperidin-4-yl, pyrrolidin-3-yl or azetidin-3-yl which are optionally substituted, preferably unsubstituted cyclopropylmethyl, unsubstituted oxetan-3-yl, unsubstituted tetrahydrofuran-3-yl; and

in which R¹, A and R³ have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
R² represents 3-hydroxybutan-2-yl, prop-2-yn-1-yl, but-2-yn-1-yl, 2,2-dimethyl-2-methoxyethyl, methoxyethyl; or
   cyclopropylmethyl, tetrahydrofuran-3-yl, tetrahydrofuran-2-ylmethyl, tetrahydrofuran-3-ylmethyl, oxetan-3-yl, tetrahydropyran-4-yl, tetrahydro-2H-pyran-4-ylmethyl, (4-methylmorpholin-2-yl)methyl, pyridin-4-yl, pyridin-3-yl, 1,3,4-thiadiazol-2-yl, 1,3-thiazol-2-yl, piperidin-4-yl, pyrrolidin-3-yl or azetidin-3-yl which are optionally substituted,
   preferably unsubstituted cyclopropylmethyl, unsubstituted oxetan-3-yl, unsubstituted (3R)-tetrahydrofuran-3-yl, unsubstituted (3S)-tetrahydrofuran-3-yl, [(2R)-4-methylmorpholin-2-yl]methyl, [(2S)-4-methylmorpholin-2-yl]methyl, (2R,3R)-3-hydroxybutan-2-yl, (2S,3S)-3-hydroxybutan-2-yl, (2S,3R)-3-hydroxybutan-2-yl or (2R,3S)-3-hydroxybutan-2-yl; and

in which R¹, A and R³ have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
R² represents cyclopropylmethyl, tetrahydrofuran-3-yl, tetrahydrofuran-2-ylmethyl, tetrahydrofuran-3-ylmethyl, prop-2-yn-1-yl, but-2-yn-1-yl, oxetan-3-yl, tetrahydropyran-4-yl, tetrahydro-2H-pyran-4-ylmethyl, pyridin-4-yl, pyridin-3-yl, 1,3,4-thiadiazol-2-yl, 1,3-thiazol-2-yl, 2,2-dimethyl-2-methoxyethyl, methoxyethyl, piperidin-4-yl, pyrrolidin-3-yl or azetidin-3-yl which are optionally substituted, preferably unsubstituted cyclopropylmethyl, unsubstituted oxetan-3-yl, unsubstituted tetrahydrofuran-3-yl; and

in which R¹, A and R³ have the same meaning as defined in general formula (Ia),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
R² represents 3-hydroxybutan-2-yl, prop-2-yn-1-yl, but-2-yn-1-yl, 2,2-dimethyl-2-methoxyethyl, methoxyethyl; or
   cyclopropylmethyl, tetrahydrofuran-3-yl, tetrahydrofuran-2-ylmethyl, tetrahydrofuran-3-ylmethyl, oxetan-3-yl, tetrahydropyran-4-yl, tetrahydro-2H-pyran-4-ylmethyl, (4-methylmorpholin-2-yl)methyl, pyridin-4-yl, pyridin-3-yl, 1,3,4-thiadiazol-2-yl, 1,3-thiazol-2-yl, piperidin-4-yl, pyrrolidin-3-yl or azetidin-3-yl which are optionally substituted,
   preferably unsubstituted cyclopropylmethyl, unsubstituted oxetan-3-yl, unsubstituted (3R)-tetrahydrofuran-3-yl, unsubstituted (3S)-tetrahydrofuran-3-yl, [(2R)-4-methylmorpholin-2-yl]methyl, [(2S)-4-methylmorpholin-2-yl]methyl, (2R,3R)-3-hydroxybutan-2-yl, (2S,3S)-3-hydroxybutan-2-yl, (2S,3R)-3-hydroxybutan-2-yl or (2R,3S)-3-hydroxybutan-2-yl; and

in which R¹, A and R³ have the same meaning as defined in general formula (Ia),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of formula (I), wherein
R² represents unsubstituted tetrahydrofuran-3-yl or unsubstituted oxetan-3-yl; and

in which R¹, A and R³ have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of formula (I), wherein
R² represents unsubstituted (3R)-tetrahydrofuran-3-yl, (3S)-tetrahydrofuran-3-yl or unsubstituted oxetan-3-yl; and

in which R¹, A and R³ have the same meaning as defined in general formula (I),
or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of formula (I), wherein
R² represents unsubstituted (3R)-tetrahydrofuran-3-yl; and

in which R¹, A and R³ have the same meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of formula (I), wherein
R² represents [(2R)-4-methylmorpholin-2-yl]methyl, (2R,3R)-3-hydroxybutan-2-yl, or (2S,3S)-3-hydroxybutan-2-yl; and

in which R¹, A and R³ have the same meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of formula (I), wherein
R² represents [(2R)-4-methylmorpholin-2-yl]methyl; and

in which R¹, A and R³ have the same meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of formula (I), wherein
R² represents (2R,3R)-3-hydroxybutan-2-yl, or (2S,3S)-3-hydroxybutan-2-yl; and

in which R¹, A and R³ have the same meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of formula (Ia), wherein
R² represents unsubstituted tetrahydrofuran-3-yl or unsubstituted oxetan-3-yl; and

in which R¹, A and R³ have the same meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of formula (Ia), wherein
R² represents unsubstituted (3R)-tetrahydrofuran-3-yl, (3S)-tetrahydrofuran-3-yl or unsubstituted oxetan-3-yl; and

in which R¹, A and R³ have the same meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of formula (Ia), wherein
R² represents unsubstituted (3R)-tetrahydrofuran-3-yl; and

in which R¹, A and R³ have the same meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of formula (Ia), wherein
R² represents [(2R)-4-methylmorpholin-2-yl]methyl, (2R,3R)-3-hydroxybutan-2-yl or (2S,3S)-3-hydroxybutan-2-yl; and

in which R¹, A and R³ have the same meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of formula (Ia), wherein
R² represents [(2R)-4-methylmorpholin-2-yl]methyl; and

in which R¹, A and R³ have the same meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of formula (Ia), wherein
R² represents (2R,3R)-3-hydroxybutan-2-yl, or (2S,3S)-3-hydroxybutan-2-yl; and

in which R¹, A and R³ have the same meaning as defined in general formula (a),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R¹ represents methyl or ethyl; and

in which R² and R³ have the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R¹ represents methyl or ethyl; and

in which R² and R³ have the meaning as defined in general formula (a),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably to compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R³ represents a methyl group; and

in which R¹ and R² have the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —C₂-C₄-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl;
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and

in which R^{c}, R¹ and R³ have the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —C₂-C₃-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl;
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and

in which R^{c}, R¹ and R³ have the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —C₂-C₄-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl;
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and

in which R^{c}, R¹ and R³ have the meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —C₂-C₃-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl;
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R; and
q represents an integer of 0; and

in which R^{c}, R¹ and R³ have the meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —(CH₂)_{q}-(4- to 6-membered heterocycloalkyl); and wherein (CH₂)_{q}-(4- to 6-membered heterocycloalkyl) is optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and
   wherein independently any ring nitrogen atom, if present in said —(CH₂)_{q}-(4 to 6-membered heterocycloalkyl) is substituted with R^{c}; and wherein said —(CH₂)_{q}-(4 to 6-membered heterocycloalkyl) is preferably —(CH₂)_{q}-morpholinyl; and
q represents an integer of 1; and

in which R^{c}, R¹ and R³ have the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —(CH₂)_{q}-morpholinyl, wherein the ring nitrogen atom is substituted with R^{c}; and
R^{c} represents methyl; and
q represents an integer of 1; and

in which R¹ and R³ have the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —(CH₂)_{q}-(4- to 6-membered heterocycloalkyl); and wherein (CH₂)_{q}-(4- to 6-membered heterocycloalkyl) is optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and
   wherein independently any ring nitrogen atom, if present in said —(CH₂)_{q}-(4 to 6-membered heterocycloalkyl) is substituted with R^{c}; and wherein said —(CH₂)_{q}-(4 to 6-membered heterocycloalkyl) is preferably —(CH₂)_{q}-morpholinyl; and
q represents an integer of 1; and

in which R^{c}, R¹ and R³ have the meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —(CH₂)_{q}-morpholinyl, wherein the ring nitrogen atom is substituted with R^{c}; and
R^{c} represents methyl; and
q represents an integer of 1; and

in which R¹ and R³ have the meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —C₂-C₄-alkyl-OH; and

in which R¹ and R³ have the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —C₂-C₄-alkyl-OH; and

in which R¹ and R³ have the meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably to compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R³ represents methyl; and

in which R² has the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

In another preferred embodiment, the invention relates to compounds of general formula (I), more preferably to compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents chloro;
R³ represents methyl; and

in which R² has the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably to compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R³ represents methyl;
R² represents —C₂-C₄-alkyl-OR⁴, CH₂—(C₃-C₄-cycloalkyl), C₃-C₄-cycloalkyl, —(CH₂)_{q}-(4 to 6-membered heterocycloalkyl), or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted one or two times, identically or differently, at any ring carbon atom with a substituent selected from C₁-C₄-alkyl, optionally substituted with 1-5 halogen atoms which are the same or different, a halogen atom, —NR^{a}R^{b} or —COOR⁵; and wherein independently any ring nitrogen atom, if present in said —(CH₂)_{q}-(4- to 6-membered heterocycloalkyl) is substituted with R^{c}; and
q represents an integer of 0; and

in which R^{c} and R¹ have the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably to compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R³ represents methyl;
R² represents —C₂-C₃-alkyl-OR⁴, CH₂—(C₃-C₄-cycloalkyl), C₃-C₄-cycloalkyl, —(CH₂)_{q}-(4 to 6-membered heterocycloalkyl), or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted one or two times, identically or differently, at any ring carbon atom with a substituent selected from C₁-C₄-alkyl, optionally substituted with 1-5 halogen atoms which are the same or different, a halogen atom, —NR^{a}R^{b} or —COOR⁵; and wherein independently any ring nitrogen atom, if present in said —(CH₂)_{q}-(4- to 6-membered heterocycloalkyl) is substituted with R^{c}; and

in which R^{c} and R¹ have the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably to compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R³ represents methyl;
R² represents —(CH₂)_{q}-(4- to 6-membered heterocycloalkyl); and wherein (CH₂)_{q}-(4- to 6-membered heterocycloalkyl) is optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and
   wherein independently any ring nitrogen atom, if present in said —(CH₂)_{q}-(4 to 6-membered heterocycloalkyl) is substituted with Rc; and wherein said —(CH₂)_{q}-(4 to 6-membered heterocycloalkyl) is preferably —(CH₂)_{q}-morpholinyl;
q represents an integer of 1; and

in which R^{c} and R¹ have the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably to compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R³ represents methyl;
R² represents —(CH₂)_{q}-morpholinyl, wherein the ring nitrogen atom is substituted with R^{c}; and
R^{c} represents methyl;
q represents an integer of 1; and

in which R¹ has the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably to compounds of general formula (Ia), wherein
A represents a 6-membered heteroaryl, in particular pyrimidinyl or pyridazinyl; wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R³ represents methyl;
R² represents C₂-C₄-alkyl-OH, preferably 3-hydroxybutan-2-yl; and

in which R¹ has the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —C₂-C₄-alkyl-OR⁴, —CH₂—(C₃-C₄-cycloalkyl), C₃-C₄-cycloalkyl, —(CH₂)_{q}-(4 to 6-membered heterocycloalkyl) or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and

in which R^{c} and R³ have the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using to compounds of general formula (Ia), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —C₂-C₄-alkyl-OR⁴, —CH₂—(C₃-C₄-cycloalkyl), C₃-C₄-cycloalkyl, —(CH₂)_{q}-(4 to 6-membered heterocycloalkyl) or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and

in which R^{c} and R³ have the meaning as defined in general formula (a),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —C₂-C₃-alkyl-OR⁴, —CH₂—(C₃-C₄-cycloalkyl), C₃-C₄-cycloalkyl, —(CH₂)_{q}-(4 to 6-membered heterocycloalkyl) or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and

in which R^{c} and R³ have the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —C₂-C₃-alkyl-OR⁴, —CH₂—(C₃-C₄-cycloalkyl), C₃-C₄-cycloalkyl, —(CH₂)_{q}-(4 to 6-membered heterocycloalkyl) or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and

in which R^{c} and R³ have the meaning as defined in general formula (a),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —(CH₂)_{q}-(4- to 6-membered heterocycloalkyl); and wherein (CH₂)_{q}-(4- to 6-membered heterocycloalkyl) is optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and
   wherein independently any ring nitrogen atom, if present in said —(CH₂)_{q}-(4 to 6-membered heterocycloalkyl) is substituted with R^{c}; and wherein said —(CH₂)_{q}-(4 to 6-membered heterocycloalkyl) is preferably —(CH₂)_{q}-morpholinyl;
q represents an integer of 1; and

in which R^{c} and R³ have the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —(CH₂)_{q}-morpholinyl, wherein the ring nitrogen atom is substituted with R^{c}; and
R^{c} represents methyl;
q represents an integer of 1; and

in which R³ has the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —(CH₂)_{q}-(4- to 6-membered heterocycloalkyl); and wherein (CH₂)_{q}-(4- to 6-membered heterocycloalkyl) is optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and
   wherein independently any ring nitrogen atom, if present in said —(CH₂)_{q}-(4 to 6-membered heterocycloalkyl) is substituted with R^{c}; and wherein said —(CH₂)_{q}-(4 to 6-membered heterocycloalkyl) is preferably —(CH₂)_{q}-morpholinyl;
q represents an integer of 1; and

in which R^{c} and R³ have the meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —(CH₂)_{q}-morpholinyl, wherein the ring nitrogen atom is substituted with R^{c}; and
R^{c} represents methyl;
q represents an integer of 1; and

in which R³ has the meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using to compounds of general formula (I), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents chloro;
R² represents C₂-C₄-alkyl-OH, preferably 3-hydroxybutan-2-yl; and

in which R³ has the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (Ia), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents chloro;
R² represents C₂-C₄-alkyl-OH, preferably 3-hydroxybutan-2-yl; and

in which R³ has the meaning as defined in general formula (Ia),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably to compounds of general formula (Ia), wherein
A represents a 6-membered heteroaryl, in particular pyrimidinyl or pyridazinyl; wherein said 6-membered heteroaryl is optionally substituted with one or two substituents which are the same or different, and selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —C₂-C₃-alkyl-OR⁴, CH₂—(C₃-C₄-cycloalkyl), C₃-C₄-cycloalkyl, —(CH₂)_{q}-(4- to 6-membered heterocycloalkyl), or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
R³ represents methyl; and
q represents an integer of 0, in which R^{c} has the meaning as defined in general formula (I), or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably to compounds of general formula (Ia), wherein
A represents a 6-membered heteroaryl, in particular pyrimidinyl or pyridazinyl; wherein said 6-membered heteroaryl is optionally substituted with one or two substituents which are the same or different, and selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —(CH₂)_{q}-(4- to 6-membered heterocycloalkyl); and wherein (CH₂)_{q}-(4- to 6-membered heterocycloalkyl) is optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and
   wherein independently any ring nitrogen atom, if present in said —(CH₂)_{q}-(4 to 6-membered heterocycloalkyl) is substituted with R^{c}; and wherein —(CH₂)_{q}-(4- to 6-membered heterocycloalkyl) is preferably —(CH₂)_{q}-morpholinyl;
R³ represents methyl; and
q represents an integer of 1, in which R^{c} has the meaning as defined in general formula (I),

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted with one or two substituents which are the same or different, and selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —(CH₂)_{q}-morpholinyl, wherein the ring nitrogen atom is substituted with R^{c}; and
R^{c} represents methyl;
R³ represents methyl; and
q represents an integer of 1,

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another embodiment of the present invention relates to a method for using compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted with one or two substituents which are the same or different, and selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents chloro;
R² represents —C₂-C₄-alkyl-OH, preferably 3-hydroxybutan-2-yl; and
R³ represents methyl,

or an isomer, enantiomer, diastereomer, racemate, hydrate, solvate, or a salt thereof, or a mixture of same for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

The use of following compounds for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma, is disclosed, namely use of
1) 3-(cyclopropylmethoxy)-5-(5-methyl-1,3-thiazol-2-yl)-N-{(1R)-1-[2-(trifluoromethyl)pyrimidin-5-yl]ethyl}benzamide
2) 3-(cyclopropylmethoxy)-N-[(6-methylpyridazin-3-yl)methyl]-5-(5-methyl-1,3-thiazol-2-yl)benzamide
3) 3-(cyclopropylmethoxy)-N-[(5-methylpyrazin-2-yl)methyl]-5-(5-methyl-1,3-thiazol-2-yl)benzamide
4) 3-(cyclopropylmethoxy)-N-[(1R)-1-(5-methylpyrazin-2-yl)ethyl]-5-(5-methyl-1,3-thiazol-2-yl)benzamide
5) N-[1-(3-chloro-5-fluoropyridin-2-yl)ethyl]-3-(cyclopropylmethoxy)-5-(5-methyl-1,3-thiazol-2-yl)benzamide
6) N-[1-(5-chloro-3-fluoropyridin-2-yl)ethyl]-3-(cyclopropylmethoxy)-5-(5-methyl-1,3-thiazol-2-yl)benzamide
7) 3-(cyclopropylmethoxy)-N-[(1R)-1-(6-methylpyridazin-3-yl)ethyl]-5-(5-methyl-1,3-thiazol-2-yl)benzamide
8) 3-(cyclopropylmethoxy)-5-(5-methyl-1,3-thiazol-2-yl)-N-{(1R)-1-[6-(trifluoromethyl)pyridazin-3-yl]ethyl}benzamide
9) 3-(cyclopropylmethoxy)-N-[(1R)-1-(2-methylpyrimidin-5-yl)ethyl]-5-(5-methyl-1,3-thiazol-2-yl)benzamide
10) 3-(5-methyl-1,3-thiazol-2-yl)-5-[(3R)-tetrahydrofuran-3-yloxy]-N-{(1R)-1-[2-(trifluoromethyl)pyrimidin-5-yl]ethyl}benzamide
11) N-[(5-methylpyrazin-2-yl)methyl]-3-(5-methyl-1,3-thiazol-2-yl)-5-[(3R)-tetrahydrofuran-3-yloxy]benzamide
12) N-[1-(3-chloro-5-fluoropyridin-2-yl)ethyl]-3-(5-methyl-1,3-thiazol-2-yl)-5-[(3R)-tetrahydrofuran-3-yloxy]benzamide
13) N-[1-(5-chloro-3-fluoropyridin-2-yl)ethyl]-3-(5-methyl-1,3-thiazol-2-yl)-5-[(3R)-tetrahydrofuran-3-yloxy]benzamide
14) N-[(1R)-1-(5-chloropyridin-2-yl)ethyl]-3-(5-methyl-1,3-thiazol-2-yl)-5-[(3R)-tetrahydrofuran-3-yloxy]benzamide
15) N-[(1R)-1-(5-methylpyrazin-2-yl)ethyl]-3-(5-methyl-1,3-thiazol-2-yl)-5-[(3R)-tetrahydrofuran-3-yloxy]benzamide
16) N-[(6-methylpyridazin-3-yl)methyl]-3-(5-methyl-1,3-thiazol-2-yl)-5-[(3R)-tetrahydrofuran-3-yloxy]benzamide
17) N-[(1R)-1-(6-methylpyridazin-3-yl)ethyl]-3-(5-methyl-1,3-thiazol-2-yl)-5-[(3R)-tetrahydrofuran-3-yloxy]benzamide
18) 3-(5-methyl-1,3-thiazol-2-yl)-5-[(3R)-tetrahydrofuran-3-yloxy]-N-{(1R)-1-[6-(trifluoromethyl)pyridazin-3-yl]ethyl}benzamide
19) 3-(5-methyl-1,3-thiazol-2-yl)-5-[(3R)-tetrahydrofuran-3-yloxy]-N-{[6-(trifluoromethyl)pyridazin-3-yl]methyl}benzamide
20) 3-(5-methyl-1,3-thiazol-2-yl)-5-[(3R)-tetrahydrofuran-3-yloxy]-N-{(1R)-1-[2-(trifluoromethyl)pyrimidin-5-yl]propyl}benzamide
21) N-[(1R)-1-(2-methylpyrimidin-5-yl)ethyl]-3-(5-methyl-1,3-thiazol-2-yl)-5-[(3R)-tetrahydrofuran-3-yloxy]benzamide
22) N-[(1R)-1-(6-methylpyridin-3-yl)ethyl]-3-(5-methyl-1,3-thiazol-2-yl)-5-[(3R)-tetrahydrofuran-3-yloxy]benzamide
23) N-[(1R)-1-(6-methylpyridazin-3-yl)ethyl]-3-(5-methyl-1,3-thiazol-2-yl)-5-[(3S)-tetrahydrofuran-3-yloxy]benzamide
24) 3-(5-methyl-1,3-thiazol-2-yl)-5-[(3S)-tetrahydrofuran-3-yloxy]-N-{(1R)-1-[2-(trifluoromethyl)pyrimidin-5-yl]ethyl}benzamide
25) N-[(1R)-1-(5-chloropyridin-2-yl)ethyl]-3-(5-methyl-1,3-thiazol-2-yl)-5-[(3S)-tetrahydrofuran-3-yloxy]benzamide
26) N-[(1R)-1-(5-methylpyridin-2-yl)ethyl]-3-(5-methyl-1,3-thiazol-2-yl)-5-[(3S)-tetrahydrofuran-3-yloxy]benzamide
27) N-[(1R)-1-(5-methylpyrazin-2-yl)ethyl]-3-(5-methyl-1,3-thiazol-2-yl)-5-[(3S)-tetrahydrofuran-3-yloxy]benzamide
28) N-[(1R)-1-(6-methylpyridin-3-yl)ethyl]-3-(5-methyl-1,3-thiazol-2-yl)-5-[(3S)-tetrahydrofuran-3-yloxy]benzamide
29) N-[(6-methylpyridazin-3-yl)methyl]-3-(5-methyl-1,3-thiazol-2-yl)-5-(prop-2-yn-1-yloxy)benzamide
30) N-[(5-chloro-3-fluoropyridin-2-yl)methyl]-3-(5-methyl-1,3-thiazol-2-yl)-5-(prop-2-yn-1-yloxy)benzamide
31) N-[(1R)-1-(6-methylpyridin-3-yl)ethyl]-3-(5-methyl-1,3-thiazol-2-yl)-5-(prop-2-yn-1-yloxy)benzamide
32) N-[(1R)-1-(5-methylpyrazin-2-yl)ethyl]-3-(5-methyl-1,3-thiazol-2-yl)-5-(prop-2-yn-1-yloxy)benzamide
33) N-[(5-methylpyrazin-2-yl)methyl]-3-(5-methyl-1,3-thiazol-2-yl)-5-(prop-2-yn-1-yloxy)benzamide
34) 3-(5-methyl-1,3-thiazol-2-yl)-5-(prop-2-yn-1-yloxy)-N-{(1R)-1-[2-(trifluoromethyl)pyrimidin-5-yl]ethyl}benzamide
35) N-[(1R)-1-(6-methylpyridazin-3-yl)ethyl]-3-(5-methyl-1,3-thiazol-2-yl)-5-(prop-2-yn-1-yloxy)benzamide
36) 3-(5-methyl-1,3-thiazol-2-yl)-5-(prop-2-yn-1-yloxy)-N-{(1R)-1-[6-(trifluoromethyl)pyridazin-3-yl]ethyl}benzamide
37) N-[(1R)-1-(2-methylpyrimidin-5-yl)ethyl]-3-(5-methyl-1,3-thiazol-2-yl)-5-(prop-2-yn-1-yloxy)benzamide
38) 3-(but-2-yn-1-yloxy)-5-(5-methyl-1,3-thiazol-2-yl)-N-{(1R)-1-[2-(trifluoromethyl)pyrimidin-5-yl]ethyl}benzamide
39) N-[(1R)-1-(5-methylpyrazin-2-yl)ethyl]-3-(5-methyl-1,3-thiazol-2-yl)-5-(oxetan-3-yloxy)benzamide
40) N-[(1R)-1-(2-methylpyrimidin-5-yl)ethyl]-3-(5-methyl-1,3-thiazol-2-yl)-5-(oxetan-3-yloxy)benzamide
41) N-[1-(5-chloro-3-fluoropyridin-2-yl)ethyl]-3-(5-methyl-1,3-thiazol-2-yl)-5-(oxetan-3-yloxy)benzamide
42) N-[(6-methylpyridazin-3-yl)methyl]-3-(5-methyl-1,3-thiazol-2-yl)-5-(oxetan-3-yloxy)benzamide
43) N-[(1R)-1-(5-chloropyridin-2-yl)ethyl]-3-(5-methyl-1,3-thiazol-2-yl)-5-(oxetan-3-yloxy)benzamide
44) N-[(1R)-1-(6-methylpyridazin-3-yl)ethyl]-3-(5-methyl-1,3-thiazol-2-yl)-5-(oxetan-3-yloxy)benzamide
45) 3-(5-methyl-1,3-thiazol-2-yl)-5-(oxetan-3-yloxy)-N-{(1R)-1-[2-(trifluoromethyl)pyrimidin-5-yl]ethyl}benzamide
46) N-[1-(3-chloro-5-fluoropyridin-2-yl)ethyl]-3-(5-methyl-1,3-thiazol-2-yl)-5-(oxetan-3-yloxy)benzamide
47) N-[(1R)-1-(6-methylpyridin-3-yl)ethyl]-3-(5-methyl-1,3-thiazol-2-yl)-5-(oxetan-3-yloxy)benzamide
48) 3-(5-methyl-1,3-thiazol-2-yl)-5-(oxetan-3-yloxy)-N-{[6-(trifluoromethyl)pyridazin-3-yl]methyl}benzamide
49) 3-(5-methyl-1,3-thiazol-2-yl)-5-(oxetan-3-yloxy)-N-{(1R)-1-[2-(trifluoromethyl)pyrimidin-5-yl]propyl}benzamide
50) N-[(6-methylpyridazin-3-yl)methyl]-3-(5-methyl-1,3-thiazol-2-yl)-5-[(2S)-tetrahydrofuran-2-ylmethoxy]benzamide
51) N-[(5-chloro-3-fluoropyridin-2-yl)methyl]-3-(5-methyl-1,3-thiazol-2-yl)-5-[(2S)-tetrahydrofuran-2-ylmethoxy]benzamide
52) N-[(5-methylpyrazin-2-yl)methyl]-3-(5-methyl-1,3-thiazol-2-yl)-5-[(2S)-tetrahydrofuran-2-ylmethoxy]benzamide
53) N-[(1R)-1-(5-methylpyrazin-2-yl)ethyl]-3-(5-methyl-1,3-thiazol-2-yl)-5-[(2S)-tetrahydrofuran-2-ylmethoxy]benzamide
54) 3-(5-methyl-1,3-thiazol-2-yl)-5-[(2S)-tetrahydrofuran-2-ylmethoxy]-N-{(1R)-1-[2-(trifluoromethyl)pyrimidin-5-yl]ethyl}benzamide
55) N-[(1R)-1-(6-methylpyridazin-3-yl)ethyl]-3-(5-methyl-1,3-thiazol-2-yl)-5-[(2S)-tetrahydrofuran-2-ylmethoxy]benzamide
56) 3-(5-methyl-1,3-thiazol-2-yl)-5-[(2S)-tetrahydrofuran-2-ylmethoxy]-N-{(1R)-1-[6-(trifluoromethyl)pyridazin-3-yl]ethyl}benzamide
57) N-[(1R)-1-(6-methylpyridazin-3-yl)ethyl]-3-(5-methyl-1,3-thiazol-2-yl)-5-[(2R)-tetrahydrofuran-2-ylmethoxy]benzamide
58) N-[(6-methylpyridazin-3-yl)methyl]-3-(5-methyl-1,3-thiazol-2-yl)-5-[(2R)-tetrahydrofuran-2-ylmethoxy]benzamide
59) 3-(5-methyl-1,3-thiazol-2-yl)-5-[(2R)-tetrahydrofuran-2-ylmethoxy]-N-{(1R)-1-[2-(trifluoromethyl)pyrimidin-5-yl]ethyl}benzamide
60) N-[(1R)-1-(5-methylpyrazin-2-yl)ethyl]-3-(5-methyl-1,3-thiazol-2-yl)-5-[(2R)-tetrahydrofuran-2-ylmethoxy]benzamide
61) N-[1-(5-chloro-3-fluoropyridin-2-yl)ethyl]-3-(5-methyl-1,3-thiazol-2-yl)-5-[(3S)-tetrahydrofuran-3-ylmethoxy]benzamide
62) N-[(6-methylpyridazin-3-yl)methyl]-3-(5-methyl-1,3-thiazol-2-yl)-5-[(3S)-tetrahydrofuran-3-ylmethoxy]benzamide
63) N-[(5-methylpyrazin-2-yl)methyl]-3-(5-methyl-1,3-thiazol-2-yl)-5-[(3S)-tetrahydrofuran-3-ylmethoxy]benzamide
64) N-[(1R)-1-(5-methylpyrazin-2-yl)ethyl]-3-(5-methyl-1,3-thiazol-2-yl)-5-[(3S)-tetrahydrofuran-3-ylmethoxy]benzamide
65) N-[1-(3-chloro-5-fluoropyridin-2-yl)ethyl]-3-(5-methyl-1,3-thiazol-2-yl)-5-[(3R)-tetrahydrofuran-3-ylmethoxy]benzamide
66) 3-(5-methyl-1,3-thiazol-2-yl)-5-[(3S)-tetrahydrofuran-3-ylmethoxy]-N-{(1R)-1-[2-(trifluoromethyl)pyrimidin-5-yl]ethyl}benzamide
67) N-[(1R)-1-(6-methylpyridazin-3-yl)ethyl]-3-(5-methyl-1,3-thiazol-2-yl)-5-[(3S)-tetrahydrofuran-3-ylmethoxy]benzamide
68) 3-(5-methyl-1,3-thiazol-2-yl)-5-[(3S)-tetrahydrofuran-3-ylmethoxy]-N-{(1R)-1-[6-(trifluoromethyl)pyridazin-3-yl]ethyl}benzamide
69) 3-(5-methyl-1,3-thiazol-2-yl)-5-[(3R)-tetrahydrofuran-3-ylmethoxy]-N-{(1R)-1-[2-(trifluoromethyl)pyrimidin-5-yl]ethyl}benzamide
70) N-[(1R)-1-(5-methylpyrazin-2-yl)ethyl]-3-(5-methyl-1,3-thiazol-2-yl)-5-[(3R)-tetrahydrofuran-3-ylmethoxy]benzamide
71) N-[(6-methylpyridazin-3-yl)methyl]-3-(5-methyl-1,3-thiazol-2-yl)-5-[(3R)-tetrahydrofuran-3-ylmethoxy]benzamide
72) N-[(5-methylpyrazin-2-yl)methyl]-3-(5-methyl-1,3-thiazol-2-yl)-5-[(3R)-tetrahydrofuran-3-ylmethoxy]benzamide
73) N-[(1R)-1-(6-methylpyridazin-3-yl)ethyl]-3-(5-methyl-1,3-thiazol-2-yl)-5-[(3R)-tetrahydrofuran-3-ylmethoxy]benzamide
74) N-[1-(5-chloro-3-fluoropyridin-2-yl)ethyl]-3-(5-methyl-1,3-thiazol-2-yl)-5-[(3R)-tetrahydrofuran-3-ylmethoxy] benzamide
75) 3-(5-methyl-1,3-thiazol-2-yl)-5-[(3R)-tetrahydrofuran-3-ylmethoxy]-N-{(1R)-1-[6-(trifluoromethyl)pyridazin-3-yl]ethyl}benzamide
76) N-[1-(3-chloro-5-fluoropyridin-2-yl)ethyl]-3-(5-methyl-1,3-thiazol-2-yl)-5-(tetrahydro-2H-pyran-4-yloxy)benzamide
77) N-[1-(5-chloro-3-fluoropyridin-2-yl)ethyl]-3-(5-methyl-1,3-thiazol-2-yl)-5-(tetrahydro-2H-pyran-4-yloxy)benzamide
78) N-[(6-methylpyridazin-3-yl)methyl]-3-(5-methyl-1,3-thiazol-2-yl)-5-(tetrahydro-2H-pyran-4-yloxy)benzamide
79) N-[(5-methylpyrazin-2-yl)methyl]-3-(5-methyl-1,3-thiazol-2-yl)-5-(tetrahydro-2H-pyran-4-yloxy)benzamide
80) N-[(1R)-1-(5-methylpyrazin-2-yl)ethyl]-3-(5-methyl-1,3-thiazol-2-yl)-5-(tetrahydro-2H-pyran-4-yloxy)benzamide
81) 3-(5-methyl-1,3-thiazol-2-yl)-5-(tetrahydro-2H-pyran-4-yloxy)-N-{(1R)-1-[2-(trifluoromethyl)pyrimidin-5-yl]ethyl}benzamide
82) N-[(1R)-1-(6-methoxypyridin-3-yl)ethyl]-3-(5-methyl-1,3-thiazol-2-yl)-5-(tetrahydro-2H-pyran-4-yloxy)benzamide
83) N-[(1R)-1-(6-methylpyridazin-3-yl)ethyl]-3-(5-methyl-1,3-thiazol-2-yl)-5-(tetrahydro-2H-pyran-4-yloxy)benzamide
84) N-[(6-methoxypyridazin-3-yl)methyl]-3-(5-methyl-1,3-thiazol-2-yl)-5-(tetrahydro-2H-pyran-4-yloxy)benzamide
85) 3-(5-methyl-1,3-thiazol-2-yl)-5-(tetrahydro-2H-pyran-4-yloxy)-N-{(1R)-1-[6-(trifluoromethyl)pyridazin-3-yl]ethyl}benzamide
86) 3-(5-methyl-1,3-thiazol-2-yl)-5-(tetrahydro-2H-pyran-4-yloxy)-N-{(1R)-1-[2-(trifluoromethyl)pyrimidin-5-yl]propyl}benzamide
87) N-[(1R)-1-(6-methylpyridin-3-yl)ethyl]-3-(5-methyl-1,3-thiazol-2-yl)-5-(tetrahydro-2H-pyran-4-yloxy)benzamide
88) N-[(6-methylpyridazin-3-yl)methyl]-3-(5-methyl-1,3-thiazol-2-yl)-5-(tetrahydro-2H-pyran-4-ylmethoxy)benzamide
89) N-[(1R)-1-(5-methylpyrazin-2-yl)ethyl]-3-(5-methyl-1,3-thiazol-2-yl)-5-(tetrahydro-2H-pyran-4-ylmethoxy)benzamide
90) N-[1-(5-chloro-3-fluoropyridin-2-yl)ethyl]-3-(5-methyl-1,3-thiazol-2-yl)-5-(tetrahydro-2H-pyran-4-ylmethoxy)benzamide
91) N-[1-(3-chloro-5-fluoropyridin-2-yl)ethyl]-3-(5-methyl-1,3-thiazol-2-yl)-5-(tetrahydro-2H-pyran-4-ylmethoxy)benzamide
92) N-[(5-methylpyrazin-2-yl)methyl]-3-(5-methyl-1,3-thiazol-2-yl)-5-(tetrahydro-2H-pyran-4-ylmethoxy)benzamide
93) 3-(5-methyl-1,3-thiazol-2-yl)-5-(tetrahydro-2H-pyran-4-ylmethoxy)-N-{(1R)-1-[2-(trifluoromethyl)pyrimidin-5-yl]ethyl}benzamide
94) N-[(1R)-1-(6-methylpyridazin-3-yl)ethyl]-3-(5-methyl-1,3-thiazol-2-yl)-5-(tetrahydro-2H-pyran-4-ylmethoxy)benzamide
95) N-[(1R)-1-(6-methylpyridazin-3-yl)ethyl]-3-[(2-methylpyridin-4-yl)oxy]-5-(5-methyl-1,3-thiazol-2-yl)benzamide
96) N-[(6-methylpyridazin-3-yl)methyl]-3-[(2-methylpyridin-4-yl)oxy]-5-(5-methyl-1,3-thiazol-2-yl)benzamide
97) N-[(1R)-1-(5-methylpyrazin-2-yl)ethyl]-3-[(2-methylpyridin-4-yl)oxy]-5-(5-methyl-1,3-thiazol-2-yl)benzamide
98) 3-[(2-methylpyridin-4-yl)oxy]-5-(5-methyl-1,3-thiazol-2-yl)-N-{(1R)-1-[2-(trifluoromethyl)pyrimidin-5-yl]ethyl}benzamide
99) N-[(5-methylpyrazin-2-yl)methyl]-3-[(2-methylpyridin-4-yl)oxy]-5-(5-methyl-1,3-thiazol-2-yl)benzamide
100) 3-[(2-methylpyridin-4-yl)oxy]-5-(5-methyl-1,3-thiazol-2-yl)-N-{(1R)-1-[6-(trifluoromethyl)pyridazin-3-yl]ethyl}benzamide
101) 3-[(2-methylpyridin-4-yl)oxy]-N-[(1R)-1-(2-methylpyrimidin-5-yl)ethyl]-5-(5-methyl-1,3-thiazol-2-yl)benzamide
102) N-[(1R)-1-(6-methylpyridin-3-yl)ethyl]-3-[(2-methylpyridin-4-yl)oxy]-5-(5-methyl-1,3-thiazol-2-yl)benzamide
103) 3-[(6-methylpyridin-3-yl)oxy]-5-(5-methyl-1,3-thiazol-2-yl)-N-{(1R)-1-[2-(trifluoromethyl)pyrimidin-5-yl]ethyl}benzamide
104) N-[(1R)-1-(5-methylpyrazin-2-yl)ethyl]-3-[(5-methyl-1,3,4-thiadiazol-2-yl)oxy]-5-(5-methyl-1,3-thiazol-2-yl)benzamide
105) N-[(1R)-1-(6-methylpyridazin-3-yl)ethyl]-3-[(5-methyl-1,3,4-thiadiazol-2-yl)oxy]-5-(5-methyl-1,3-thiazol-2-yl)benzamide
106) 3-[(5-methyl-1,3,4-thiadiazol-2-yl)oxy]-5-(5-methyl-1,3-thiazol-2-yl)-N-{(1R)-1-[2-(trifluoromethyl)pyrimidin-5-yl]ethyl}benzamide
107) N-[(1R)-1-(5-methylpyrazin-2-yl)ethyl]-3-(5-methyl-1,3-thiazol-2-yl)-5-(1,3-thiazol-2-yloxy)benzamide
108) N-[(1R)-1-(6-methylpyridazin-3-yl)ethyl]-3-(5-methyl-1,3-thiazol-2-yl)-5-(1,3-thiazol-2-yloxy)benzamide
109) N-[(1R)-1-(6-methylpyridin-3-yl)ethyl]-3-(5-methyl-1,3-thiazol-2-yl)-5-(1,3-thiazol-2-yloxy)benzamide
110) N-[(1R)-1-(5-chloropyridin-2-yl)ethyl]-3-(5-chloro-1,3-thiazol-2-yl)-5-(2-methoxy-2-methylpropoxy)benzamide
111) 3-(5-chloro-1,3-thiazol-2-yl)-5-(2-methoxy-2-methylpropoxy)-N-{(1R)-1-[2-(trifluoromethyl)pyrimidin-5-yl]ethyl}benzamide
112) 3-(5-chloro-1,3-thiazol-2-yl)-5-(2-methoxy-2-methylpropoxy)-N-[(1R)-1-(5-methylpyrazin-2-yl)ethyl]benzamide
113) N-[(6-methylpyridazin-3-yl)methyl]-3-(tetrahydro-2H-pyran-4-ylmethoxy)-5-[5-(trifluoromethyl)-1,3-thiazol-2-yl]benzamide
114) 3-(5-cyclobutyl-1,3-thiazol-2-yl)-N-[(6-methylpyridazin-3-yl)methyl]-5-(tetrahydro-2H-pyran-4-ylmethoxy)benzamide
115) 3-(5-cyclobutyl-1,3-thiazol-2-yl)-5-(tetrahydro-2H-pyran-4-ylmethoxy)-N-{(1R)-1-[2-(trifluoromethyl)pyrimidin-5-yl]ethyl}benzamide
116) 3-(5-cyclobutyl-1,3-thiazol-2-yl)-N-[(1R)-1-(5-methylpyrazin-2-yl)ethyl]-5-[(3S)-tetrahydrofuran-3-ylmethoxy]benzamide
117) 3-(5-cyclobutyl-1,3-thiazol-2-yl)-N-[(6-methylpyridazin-3-yl)methyl]-5-[(3S)-tetrahydrofuran-3-ylmethoxy]benzamide
118) 3-(5-cyclobutyl-1,3-thiazol-2-yl)-5-[(3S)-tetrahydrofuran-3-ylmethoxy]-N-{(1R)-1-[2-(trifluoromethyl)pyrimidin-5-yl]ethyl}benzamide
119) 3-(5-cyclobutyl-1,3-thiazol-2-yl)-N-[(1R)-1-(2-methylpyrimidin-5-yl)ethyl]-5-(tetrahydro-2H-pyran-4-ylmethoxy)benzamide
120) 3-(5-ethyl-1,3-thiazol-2-yl)-N-[(1R)-1-(2-methylpyrimidin-5-yl)ethyl]-5-(tetrahydro-2H-pyran-4-ylmethoxy)benzamide
121) N-[(1R)-1-(2-methylpyrimidin-5-yl)ethyl]-3-[5-(propan-2-yl)-1,3-thiazol-2-yl]-5-(tetrahydro-2H-pyran-4-ylmethoxy)benzamide
122) 3-(5-cyclobutyl-1,3-thiazol-2-yl)-N-[(1R)-1-(6-methylpyridazin-3-yl)ethyl]-5-(tetrahydro-2H-pyran-4-yloxy)benzamide
123) 3-(5-ethyl-1,3-thiazol-2-yl)-N-[(1R)-1-(6-methylpyridazin-3-yl)ethyl]-5-(tetrahydro-2H-pyran-4-yloxy)benzamide
124) 3-(5-ethyl-1,3-thiazol-2-yl)-5-(tetrahydro-2H-pyran-4-yloxy)-N-{(1R)-1-[2-(trifluoromethyl)pyrimidin-5-yl]ethyl}benzamide
125) N-[(1R)-1-(2-methylpyrimidin-5-yl)ethyl]-3-(5-methyl-1,3-thiazol-2-yl)-5-[(3S)-tetrahydrofuran-3-yloxy]benzamide
126) N-[(1R)-1-(5-methylpyrazin-2-yl)ethyl]-3-[(6-methylpyridin-3-yl)oxy]-5-(5-methyl-1,3-thiazol-2-yl)benzamide
127) N-[1-(3-chloro-5-fluoropyridin-2-yl)ethyl]-3-(5-methyl-1,3-thiazol-2-yl)-5-[(3S)-tetrahydrofuran-3-yloxy]benzamide
128) N-[(6-methylpyridazin-3-yl)methyl]-3-(5-methyl-1,3-thiazol-2-yl)-5-[(3S)-tetrahydrofuran-3-yloxy]benzamide
129) N-[1-(5-chloro-3-fluoropyridin-2-yl)ethyl]-3-(5-methyl-1,3-thiazol-2-yl)-5-[(3S)-tetrahydrofuran-3-yloxy]benzamide
130) 3-(5-cyclobutyl-1,3-thiazol-2-yl)-N-[(1R)-1-(6-methylpyridazin-3-yl)ethyl]-5-[(3S)-tetrahydrofuran-3-ylmethoxy]benzamide
131) 3-(2-methoxyethoxy)-N-[(1R)-1-(2-methylpyrimidin-5-yl)ethyl]-5-(5-methyl-1,3-thiazol-2-yl)benzamide
132) tert-butyl 4-[3-(5-methyl-1,3-thiazol-2-yl)-5-({(1R)-1-[2-(trifluoromethyl)pyrimidin-5-yl]ethyl}carbamoyl)phenoxy]piperidine-1-carboxylate
133) 3-(5-methyl-1,3-thiazol-2-yl)-5-(piperidin-4-yloxy)-N-{(1R)-1-[2-(trifluoromethyl)pyrimidin-5-yl]ethyl}benzamide
134) 3-[(1-methylpiperidin-4-yl)oxy]-5-(5-methyl-1,3-thiazol-2-yl)-N-{(1R)-1-[2-(trifluoromethyl)pyrimidin-5-yl]ethyl}benzamide
135) 3-(5-methyl-1,3-thiazol-2-yl)-5-{[1-(propan-2-yl)piperidin-4-yl]oxy}-N-{(1R)-1-[2-(trifluoromethyl)pyrimidin-5-yl]ethyl}benzamide
136) 3-{[(3R)-1-methylpyrrolidin-3-yl]oxy}-5-(5-methyl-1,3-thiazol-2-yl)-N-{(1R)-1-[2-(trifluoromethyl)pyrimidin-5-yl]ethyl}benzamide
137) 3-{[(3S)-1-methylpyrrolidin-3-yl]oxy}-5-(5-methyl-1,3-thiazol-2-yl)-N-{(1R)-1-[2-(trifluoromethyl)pyrimidin-5-yl]ethyl}benzamide
138) 3-[(1-methylazetidin-3-yl)oxy]-5-(5-methyl-1,3-thiazol-2-yl)-N-{(1R)-1-[2-(trifluoromethyl)pyrimidin-5-yl]ethyl}benzamide
139) 3-(5-methyl-1,3-thiazol-2-yl)-5-(prop-2-yn-1-yloxy)-N-{(1S)-1-[2-(trifluoromethyl)pyrimidin-5-yl]ethyl}benzamide
140) 3-(5-methyl-1,3-thiazol-2-yl)-5-(tetrahydro-2H-pyran-4-yloxy)-N-{(1S)-1-[2-(trifluoromethyl)pyrimidin-5-yl]ethyl}benzamide
141) tert-butyl 6-[3-(5-methyl-1,3-thiazol-2-yl)-5-({(1R)-1-[2-(trifluoromethyl)pyrimidin-5-yl]ethyl}carbamoyl)phenoxy]-2-azaspiro[3.3]heptane-2-carboxylate
142) 3-(5-methyl-1,3-thiazol-2-yl)-5-[(3R)-tetrahydrofuran-3-yloxy]-N-{(1R)-1-[5-(trifluoromethyl)pyrazin-2-yl]ethyl}benzamide
143) 3-(5-methyl-1,3-thiazol-2-yl)-5-(oxetan-3-yloxy)-N-{(1R)-1-[6-(trifluoromethyl)pyridin-3-yl]ethyl}benzamide

Also disclosed are the following compounds, namely:
144) 3-(1-azabicyclo[2.2.2]oct-4-yloxy)-5-(5-methyl-1,3-thiazol-2-yl)-N-{(1R)-1-[2-(trifluoromethyl)pyrimidin-5-yl]ethyl}benzamide
145) 3-[(1-acetylpiperidin-4-yl)oxy]-5-(5-methyl-1,3-thiazol-2-yl)-N-{(1R)-1-[2-(trifluoromethyl)pyrimidin-5-yl]ethyl}benzamide
146) N-{(1R)-1-[2-(difluoromethyl)pyrimidin-5-yl]ethyl}-3-(5-methyl-1,3-thiazol-2-yl)-5-[(3S)-tetrahydrofuran-3-yloxy]benzamide
147) N-{(1R)-1-[2-(difluoromethyl)pyrimidin-5-yl]ethyl}-3-(5-methyl-1,3-thiazol-2-yl)-5-(oxetan-3-yloxy)benzamide
148) N-{(1R)-1-[2-(difluoromethyl)pyrimidin-5-yl]ethyl}-3-(5-methyl-1,3-thiazol-2-yl)-5-[(3R)-tetrahydrofuran-3-yloxy]benzamide
149) N-{(1R)-1-[2-(difluoromethyl)pyrimidin-5-yl]ethyl}-3-(5-methyl-1,3-thiazol-2-yl)-5-(tetrahydro-2H-pyran-4-yloxy)benzamide
150) 3-{[(3S)-1-methylpiperidin-3-yl]oxy}-5-(5-methyl-1,3-thiazol-2-yl)-N-{(1R)-1-[2-(trifluoromethyl)pyrimidin-5-yl]ethyl}benzamide
151) 3-[(3-methyloxetan-3-yl)oxy]-5-(5-methyl-1,3-thiazol-2-yl)-N-{(1R)-1-[2-(trifluoromethyl)pyrimidin-5-yl]ethyl}benzamide
152) 3-(5-methyl-1,3-thiazol-2-yl)-5-[(3R)-tetrahydrofuran-3-yloxy]-N-{(1R)-1-[6-(trifluoromethyl)pyridin-3-yl]ethyl}benzamide
153) 3-{[(3R)-1-methylpiperidin-3-yl]oxy}-5-(5-methyl-1,3-thiazol-2-yl)-N-{(1R)-1-[2-(trifluoromethyl)pyrimidin-5-yl]ethyl}benzamide
154) 3-(5-methyl-1,3-thiazol-2-yl)-5-[2-(1H-1,2,4-triazol-1-yl)ethoxy]-N-{(1R)-1-[2-(trifluoromethyl)pyrimidin-5-yl]ethyl}benzamide
155) 3-(5-methyl-1,3-thiazol-2-yl)-5-[2-(1H-1,2,4-triazol-1-yl)ethoxy]-N-{(1R)-1-[6-(trifluoromethyl)pyridin-3-yl]ethyl}benzamide
156) 3-(5-methyl-1,3-thiazol-2-yl)-5-(oxetan-3-yloxy)-N-{(1R)-1-[6-(trifluoromethyl)pyridazin-3-yl]ethyl}benzamide
157) Trans Isomer 1; 3-{[3-hydroxybutan-2-yl]oxy}-5-(5-methyl-1,3-thiazol-2-yl)-N-{(1R)-1-[2-(trifluoromethyl)pyrimidin-5-yl]ethyl}benzamide
158) Trans Isomer 2; 3-{[3-hydroxybutan-2-yl]oxy}-5-(5-methyl-1,3-thiazol-2-yl)-N-{(1R)-1-[2-(trifluoromethyl)pyrimidin-5-yl]ethyl}benzamide
159) N-{(1R)-1-[6-(difluoromethyl)pyridin-3-yl]ethyl}-3-(5-methyl-1,3-thiazol-2-yl)-5-(oxetan-3-yloxy)benzamide
160) 3-{[trans-3-(dimethylamino)cyclobutyl]oxy}-5-(5-methyl-1,3-thiazol-2-yl)-N-{(1R)-1-[2-(trifluoromethyl)pyrimidin-5-yl]ethyl}benzamide
161) 3-(5-methyl-1,3-thiazol-2-yl)-5-[(3R)-tetrahydrofuran-3-yloxy]-N-{[2-(trifluoromethyl)pyrimidin-5-yl]methyl}benzamide
162) 3-(5-methyl-1,3-thiazol-2-yl)-5-(oxetan-3-yloxy)-N-{[2-(trifluoromethyl)pyrimidin-5-yl]methyl}benzamide
163) 3-[(3R)-1-azabicyclo[2.2.2]oct-3-yloxy]-5-(5-methyl-1,3-thiazol-2-yl)-N-{(1R)-1-[2-(trifluoromethyl)pyrimidin-5-yl]ethyl}benzamide
164) 3-(5-ethyl-1,3-thiazol-2-yl)-5-(oxetan-3-yloxy)-N-{(1R)-1-[2-(trifluoromethyl)pyrimidin-5-yl]ethyl}benzamide
165) 3-[(6-methylpyridazin-3-yl)oxy]-5-(5-methyl-1,3-thiazol-2-yl)-N-{(1R)-1-[2-(trifluoromethyl)pyrimidin-5-yl]ethyl}benzamide
166) N-{(1R)-1-[6-(difluoromethyl)pyridin-3-yl]ethyl}-3-(5-methyl-1,3-thiazol-2-yl)-5-[(3R)-tetrahydrofuran-3-yloxy]benzamide
167) 3-[(3R)-1-azabicyclo[2.2.2]oct-3-yloxy]-5-(5-methyl-1,3-thiazol-2-yl)-N-{(1R)-1-[6-(trifluoromethyl)pyridin-3-yl]ethyl}benzamide
168) 3-[(3S)-1-azabicyclo[2.2.2]oct-3-yloxy]-5-(5-ethyl-1,3-thiazol-2-yl)-N-{(1R)-1-[2-(trifluoromethyl)pyrimidin-5-yl]ethyl}benzamide
169) 3-[(3R)-1-azabicyclo[2.2.2]oct-3-yloxy]-5-(5-ethyl-1,3-thiazol-2-yl)-N-{(1R)-1-[2-(trifluoromethyl)pyrimidin-5-yl]ethyl}benzamide
170) 3-[(3S)-1-azabicyclo[2.2.2]oct-3-yloxy]-5-(5-methyl-1,3-thiazol-2-yl)-N-{(1R)-1-[6-(trifluoromethyl)pyridin-3-yl]ethyl}benzamide
171) 3-[(5-methyl-1,3,4-thiadiazol-2-yl)oxy]-5-(5-methyl-1,3-thiazol-2-yl)-N-{(1R)-1-[6-(trifluoromethyl)pyridin-3-yl]ethyl}benzamide
172) 3-[(2R)-1,4-dioxan-2-ylmethoxy]-5-(5-methyl-1,3-thiazol-2-yl)-N-{(1R)-1-[6-(trifluoromethyl)pyridin-3-yl]ethyl}benzamide
173) 3-[(2R)-1,4-dioxan-2-ylmethoxy]-5-(5-methyl-1,3-thiazol-2-yl)-N-{(1R)-1-[2-(trifluoromethyl)pyrimidin-5-yl]ethyl}benzamide
174) 3-[(2R)-1,4-dioxan-2-ylmethoxy]-5-(5-methyl-1,3-thiazol-2-yl)-N-{(1R)-1-[6-(trifluoromethyl)pyridazin-3-yl]ethyl}benzamide
175) 3-[(2S)-1,4-dioxan-2-ylmethoxy]-5-(5-methyl-1,3-thiazol-2-yl)-N-{(1R)-1-[6-(trifluoromethyl)pyridin-3-yl]ethyl}benzamide
176) 3-[(2S)-1,4-dioxan-2-ylmethoxy]-5-(5-methyl-1,3-thiazol-2-yl)-N-{(1R)-1-[2-(trifluoromethyl)pyrimidin-5-yl]ethyl}benzamide
177) 3-[(2S)-1,4-dioxan-2-ylmethoxy]-5-(5-methyl-1,3-thiazol-2-yl)-N-{(1R)-1-[6-(trifluoromethyl)pyridazin-3-yl]ethyl}benzamide
178) Trans Isomer 1; 3-{[3-hydroxybutan-2-yl]oxy}-5-(5-methyl-1,3-thiazol-2-yl)-N-{(1R)-1-[6-(trifluoromethyl)pyridazin-3-yl]ethyl}benzamide
179) Trans Isomer 1; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide
180) Cis Isomer 1; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide
181) Trans Isomer 1; 3-(5-chloro-1,3-thiazol-2-yl)-5-{[3-hydroxybutan-2-yl]oxy}-N-{(1R)-1-[6-(trifluoromethyl)pyridin-3-yl]ethyl}benzamide
182) Cis Isomer 2; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide
183) Trans Isomer 2; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide
184) Trans Isomer 2; 3-(5-chloro-1,3-thiazol-2-yl)-5-{[3-hydroxybutan-2-yl]oxy}-N-{(1R)-1-[6-(trifluoromethyl)pyridin-3-yl]ethyl}benzamide
185) tert-Butyl (3R)-3-[3-(5-methyl-1,3-thiazol-2-yl)-5-({(1R)-1-[2-(trifluoromethyl)pyrimidin-5-yl]ethyl}carbamoyl)phenoxy]piperidine-1-carboxylate, as a mixture of diastereoisomers
186) 3-(but-2-yn-1-yloxy)-N-[(1R)-1-(6-methylpyridazin-3-yl)ethyl]-5-(5-methyl-1,3-thiazol-2-yl)benzamide
187) 3-[(3S)-1-azabicyclo[2.2.2]oct-3-yloxy]-5-(5-methyl-1,3-thiazol-2-yl)-N-{(1R)-1-[2-(trifluoromethyl)pyrimidin-5-yl]ethyl}benzamide
188) 3-(5-methyl-1,3-thiazol-2-yl)-5-(piperidin-4-yloxy)-N-{(1R)-1-[6-(trifluoromethyl)pyridazin-3-yl]ethyl}benzamide
189) 3-(2-azaspiro[3.3]hept-6-yloxy)-5-(5-methyl-1,3-thiazol-2-yl)-N-{(1R)-1-[2-(trifluoromethyl)pyrimidin-5-yl]ethyl}benzamide
190) 3-(5-methyl-1,3-thiazol-2-yl)-5-[(3S)-pyrrolidin-3-yloxy]-N-{(1R)-1-[2-(trifluoromethyl)pyrimidin-5-yl]ethyl}benzamide
191) 3-{[3-fluoropiperidin-4-yl]oxy}-5-(5-methyl-1,3-thiazol-2-yl)-N-{(1R)-1-[2-(trifluoromethyl)pyrimidin-5-yl]ethyl}benzamide, as a mixture of cis isomers
192) Diastereoisomer 1; 3-(5-methyl-1,3-thiazol-2-yl)-5-(piperidin-3-yloxy)-N-{(1R)-1-[2-(trifluoromethyl)pyrimidin-5-yl]ethyl}benzamide
193) Diastereoisomer 2; 3-(5-methyl-1,3-thiazol-2-yl)-5-(piperidin-3-yloxy)-N-{(1R)-1-[2-(trifluoromethyl)pyrimidin-5-yl]ethyl}benzamide
194) Cis Isomer 1; 3-(5-methyl-1,3-thiazol-2-yl)-5-{[2-(trifluoromethyl)piperidin-4-yl]oxy}-N-{(1R)-1-[2-(trifluoromethyl)pyrimidin-5-yl]ethyl}benzamide
195) Cis Isomer 2; 3-(5-methyl-1,3-thiazol-2-yl)-5-{[2-(trifluoromethyl)piperidin-4-yl]oxy}-N-{(1R)-1-[2-(trifluoromethyl)pyrimidin-5-yl]ethyl}benzamide
196) 3-{[2-methyl-2-azabicyclo[2.2.1]hept-5-yl]oxy}-5-(5-methyl-1,3-thiazol-2-yl)-N-{(1R)-1-[2-(trifluoromethyl)pyrimidin-5-yl]ethyl}benzamide
197) 3-[(1-methylpiperidin-4-yl)oxy]-5-(5-methyl-1,3-thiazol-2-yl)-N-{(1R)-1-[6-(trifluoromethyl)pyridazin-3-yl]ethyl}benzamide
198) 3-[(1-methylazetidin-3-yl)oxy]-5-(5-methyl-1,3-thiazol-2-yl)-N-{(1R)-1-[6-(trifluoromethyl)pyridazin-3-yl]ethyl}benzamide
199) 3-[(3-fluoro-1-methylpiperidin-4-yl)oxy]-5-(5-methyl-1,3-thiazo-2-yl)-N-{(1R)-1-[2-(trifluoromethyl)pyrimidin-5-yl]ethyl}benzamide, as a single unknown isomer 200) 3-{[1-(dimethylamino)cyclopropyl]methoxy}-5-(5-methyl-1,3-thiazol-2-yl)-N-{(1R)-1-[2-(trifluoromethyl)pyrimidin-5-yl]ethyl}benzamide
201) 3-[(2-methyl-2-azaspiro[3.3]hept-6-yl)oxy]-5-(5-methyl-1,3-thiazol-2-yl)-N-{(1R)-1-[2-(trifluoromethyl)pyrimidin-5-yl]ethyl}benzamide
202) N-{(1R)-1-[2-(difluoromethyl)pyrimidin-5-yl]ethyl}-3-[(1-methylpiperidin-4-yl)oxy]-5-(5-methyl-1,3-thiazol-2-yl)benzamide
203) 3-{[(3-endo)-8-methyl-8-azabicyclo[3.2.1]oct-3-yl]oxy}-5-(5-methyl-1,3-thiazol-2-yl)-N-{(1R)-1-[2-(trifluoromethyl)pyrimidin-5-yl]ethyl}benzamide
204) 3-{[(3-exo)-8-methyl-8-azabicyclo[3.2.1]oct-3-yl]oxy}-5-(5-methyl-1,3-thiazol-2-yl)-N-{(1R)-1-[2-(trifluoromethyl)pyrimidin-5-yl]ethyl}benzamide
205) 3-{[(4aS,7R,7aR)-4-methyloctahydrocyclopenta[b][1,4]oxazin-7-yl]oxy}-5-(5-methyl-1,3-thiazol-2-yl)-N-{(1R)-1-[2-(trifluoromethyl)pyrimidin-5-yl]ethyl}benzamide
206) 3-{[(4aS,7S,7aR)-4-methyloctahydrocyclopenta[b][1,4]oxazin-7-yl]oxy}-5-(5-methyl-1,3-thiazol-2-yl)-N-{(1R)-1-[2-(trifluoromethyl)pyrimidin-5-yl]ethyl}benzamide
207) Diastereoisomer 1; 3-[(1-methylpiperidin-3-yl)oxy]-5-(5-methyl-1,3-thiazol-2-yl)-N-{(1R)-1-[2-(trifluoromethyl)pyrimidin-5-yl]ethyl}benzamide
208) Diastereoisomer 2; 3-[(1-methylpiperidin-3-yl)oxy]-5-(5-methyl-1,3-thiazol-2-yl)-N-{(1R)-1-[2-(trifluoromethyl)pyrimidin-5-yl]ethyl}benzamide
209) Cis Isomer 1; 3-(5-methyl-1,3-thiazol-2-yl)-5-{[1-methyl-2-(trifluoromethyl)piperidin-4-yl]oxy}-N-{(1R)-1-[2-(trifluoromethyl)pyrimidin-5-yl]ethyl}benzamide
210) Cis Isomer 2; 3-(5-methyl-1,3-thiazol-2-yl)-5-{[1-methyl-2-(trifluoromethyl)piperidin-4-yl]oxy}-N-{(1R)-1-[2-(trifluoromethyl)pyrimidin-5-yl]ethyl}benzamide
211) 3-(5-methyl-1,3-thiazol-2-yl)-5-{[1-(propan-2-yl)piperidin-4-yl]oxy}-N-{(1R)-1-[6-(trifluoromethyl)pyridazin-3-yl]ethyl}benzamide
212) 3-(5-methyl-1,3-thiazol-2-yl)-5-{[(3S)-1-(propan-2-yl)pyrrolidin-3-yl]oxy}-N-{(1R)-1-[2-(trifluoromethyl)pyrimidin-5-yl]ethyl}benzamide
213) methyl 4-[3-(5-methyl-1,3-thiazol-2-yl)-5-({(1R)-1-[2-(trifluoromethyl)pyrimidin-5-yl]ethyl}carbamoyl)phenoxy]piperidine-1-carboxylate
214) ethyl 4-[3-(5-methyl-1,3-thiazol-2-yl)-5-({(1R)-1-[2-(trifluoromethyl)pyrimidin-5-yl]ethyl}carbamoyl)phenoxy]piperidine-1-carboxylate
215) ethyl (3S)-3-[3-(5-methyl-1,3-thiazol-2-yl)-5-({(1R)-1-[2-(trifluoromethyl)pyrimidin-5-yl]ethyl}carbamoyl)phenoxy]pyrrolidine-1-carboxylate
216) 3-(5-methyl-1,3-thiazol-2-yl)-5-{[1-(propan-2-yl)azetidin-3-yl]oxy}-N-{(1R)-1-[2-(trifluoromethyl)pyrimidin-5-yl]ethyl}benzamide
217) Cis Isomer 1; 3-[(-3-hydroxybutan-2-yl)oxy]-5-(5-methyl-1,3-thiazol-2-yl)-N-{(1R)-1-[6-(trifluoromethyl)pyridazin-3-yl]ethyl}benzamide
218) Cis Isomer 2; 3-[(-3-hydroxybutan-2-yl)oxy]-5-(5-methyl-1,3-thiazol-2-yl)-N-{(1R)-1-[6-(trifluoromethyl)pyridazin-3-yl]ethyl}benzamide
219) 3-[(1,1-dioxidotetrahydro-2H-thiopyran-4-yl)oxy]-5-(5-methyl-1,3-thiazol-2-yl)-N-{(1R)-1-[2-(trifluoromethyl)pyrimidin-5-yl]ethyl}benzamide
220) 3-[(1,1-dioxidotetrahydro-2H-thiopyran-4-yl)oxy]-5-(5-methyl-1,3-thiazol-2-yl)-N-{(1R)-1-[6-(trifluoromethyl)pyridin-3-yl]ethyl}benzamide
221) 3-(5-ethyl-1,3-thiazol-2-yl)-N-[(1R)-1-(5-methylpyrazin-2-yl)ethyl]-5-(tetrahydro-2H-pyran-4-yloxy)benzamide
222) 3-(5-ethyl-1,3-thiazol-2-yl)-N-[(6-methylpyridazin-3-yl)methyl]-5-(tetrahydro-2H-pyran-4-yloxy)benzamide
223) 3-(5-cyclobutyl-1,3-thiazol-2-yl)-N-[(1R)-1-(5-methylpyrazin-2-yl)ethyl]-5-(tetrahydro-2H-pyran-4-yloxy)benzamide
224) 3-(5-cyclobutyl-1,3-thiazol-2-yl)-N-[(6-methylpyridazin-3-yl)methyl]-5-(tetrahydro-2H-pyran-4-yloxy)benzamide
225) 3-(5-cyclobutyl-1,3-thiazol-2-yl)-5-(tetrahydro-2H-pyran-4-yloxy)-N-{(1R)-1-[2-(trifluoromethyl)pyrimidin-5-yl]ethyl}benzamide
226) 3-(5-cyclobutyl-1,3-thiazol-2-yl)-5-[(3S)-tetrahydrofuran-3-ylmethoxy]-N-{(1S)-1-[2-(trifluoromethyl)pyrimidin-5-yl]ethyl}benzamide
227) 3-(5-ethyl-1,3-thiazol-2-yl)-N-[(1R)-1-(6-methylpyridazin-3-yl)ethyl]-5-[(3R)-tetrahydrofuran-3-yloxy]benzamide
228) 3-(5-ethyl-1,3-thiazol-2-yl)-N-[(1R)-1-(5-methylpyrazin-2-yl)ethyl]-5-[(3R)-tetrahydrofuran-3-yloxy]benzamide
229) 3-(5-ethyl-1,3-thiazol-2-yl)-5-[(3R)-tetrahydrofuran-3-yloxy]-N-{(1R)-1-[2-(trifluoromethyl)pyrimidin-5-yl]ethyl}benzamide
230) 3-(5-cyclobutyl-1,3-thiazol-2-yl)-N-[(1R)-1-(6-methylpyridazin-3-yl)ethyl]-5-[(3R)-tetrahydrofuran-3-yloxy]benzamide
231) 3-(5-cyclobutyl-1,3-thiazol-2-yl)-N-[(1R)-1-(5-methylpyrazin-2-yl)ethyl]-5-[(3R)-tetrahydrofuran-3-yloxy]benzamide
232) 3-(5-cyclobutyl-1,3-thiazol-2-yl)-5-[(3R)-tetrahydrofuran-3-yloxy]-N-{(1R)-1-[2-(trifluoromethyl)pyrimidin-5-yl]ethyl}benzamide
233) N-[(1R)-1-(6-methylpyridazin-3-yl)ethyl]-3-[5-(propan-2-yl)-1,3-thiazol-2-yl]-5-[(3R)-tetrahydrofuran-3-yloxy]benzamide
234) N-[(1R)-1-(5-methylpyrazin-2-yl)ethyl]-3-[5-(propan-2-yl)-1,3-thiazol-2-yl]-5-[(3R)-tetrahydrofuran-3-yloxy]benzamide
235) 3-[5-(propan-2-yl)-1,3-thiazol-2-yl]-5-[(3R)-tetrahydrofuran-3-yloxy]-N-{(1R)-1-[2-(trifluoromethyl)pyrimidin-5-yl]ethyl}benzamide
236) 3-(5-ethyl-1,3-thiazol-2-yl)-N-[(1R)-1-(6-methylpyridazin-3-yl)ethyl]-5-[(3S)-tetrahydrofuran-3-yloxy]benzamide
237) 3-(5-ethyl-1,3-thiazol-2-yl)-N-[(1R)-1-(5-methylpyrazin-2-yl)ethyl]-5-[(3S)-tetrahydrofuran-3-yloxy]benzamide
238) 3-(5-ethyl-1,3-thiazol-2-yl)-5-[(3S)-tetrahydrofuran-3-yloxy]-N-{(1R)-1-[2-(trifluoromethyl)pyrimidin-5-yl]ethyl}benzamide
239) 3-(5-cyclobutyl-1,3-thiazol-2-yl)-N-[(1R)-1-(6-methylpyridazin-3-yl)ethyl]-5-[(3S)-tetrahydrofuran-3-yloxy]benzamide
240) 3-(5-cyclobutyl-1,3-thiazol-2-yl)-N-[(1R)-1-(5-methylpyrazin-2-yl)ethyl]-5-[(3S)-tetrahydrofuran-3-yloxy]benzamide
241) 3-(5-cyclobutyl-1,3-thiazol-2-yl)-5-[(3S)-tetrahydrofuran-3-yloxy]-N-{(1R)-1-[2-(trifluoromethyl)pyrimidin-5-yl]ethyl}benzamide
242) N-[(1R)-1-(6-methylpyridazin-3-yl)ethyl]-3-[5-(propan-2-yl)-1,3-thiazol-2-yl]-5-[(3S)-tetrahydrofuran-3-yloxy]benzamide
243) N-[(1R)-1-(5-methylpyrazin-2-yl)ethyl]-3-[5-(propan-2-yl)-1,3-thiazol-2-yl]-5-[(3S)-tetrahydrofuran-3-yloxy]benzamide
244) 3-[5-(propan-2-yl)-1,3-thiazol-2-yl]-5-[(3S)-tetrahydrofuran-3-yloxy]-N-{(1R)-1-[2-(trifluoromethyl)pyrimidin-5-yl]ethyl}benzamide
245) 3-(5-ethyl-1,3-thiazol-2-yl)-N-[(1R)-1-(6-methylpyridazin-3-yl)ethyl]-5-[(3R)-tetrahydrofuran-3-ylmethoxy]benzamide
246) 3-(5-ethyl-1,3-thiazol-2-yl)-N-[(1R)-1-(5-methylpyrazin-2-yl)ethyl]-5-[(3R)-tetrahydrofuran-3-ylmethoxy]benzamide
247) 3-(5-ethyl-1,3-thiazol-2-yl)-5-[(3R)-tetrahydrofuran-3-ylmethoxy]-N-{(1R)-1-[2-(trifluoromethyl)pyrimidin-5-yl]ethyl}benzamide
248) 3-(5-cyclobutyl-1,3-thiazol-2-yl)-N-[(1R)-1-(6-methylpyridazin-3-yl)ethyl]-5-[(3R)-tetrahydrofuran-3-ylmethoxy]benzamide
249) 3-(5-cyclobutyl-1,3-thiazol-2-yl)-N-[(1R)-1-(5-methylpyrazin-2-yl)ethyl]-5-[(3R)-tetrahydrofuran-3-ylmethoxy]benzamide
250) 3-(5-cyclobutyl-1,3-thiazol-2-yl)-5-[(3R)-tetrahydrofuran-3-ylmethoxy]-N-{(1R)-1-[2-(trifluoromethyl)pyrimidin-5-yl]ethyl}benzamide
251) N-[(1R)-1-(6-methylpyridazin-3-yl)ethyl]-3-[5-(propan-2-yl)-1,3-thiazol-2-yl]-5-[(3R)-tetrahydrofuran-3-ylmethoxy]benzamide
252) N-[(1R)-1-(5-methylpyrazin-2-yl)ethyl]-3-[5-(propan-2-yl)-1,3-thiazol-2-yl]-5-[(3R)-tetrahydrofuran-3-ylmethoxy]benzamide
253) 3-[5-(propan-2-yl)-1,3-thiazol-2-yl]-5-[(3R)-tetrahydrofuran-3-ylmethoxy]-N-{(1R)-1-[2-(trifluoromethyl)pyrimidin-5-yl]ethyl}benzamide
254) 3-(5-ethyl-1,3-thiazol-2-yl)-N-[(1R)-1-(6-methylpyridazin-3-yl)ethyl]-5-[(2R)-tetrahydrofuran-2-ylmethoxy]benzamide
255) 3-(5-ethyl-1,3-thiazol-2-yl)-N-[(1R)-1-(5-methylpyrazin-2-yl)ethyl]-5-[(2R)-tetrahydrofuran-2-ylmethoxy]benzamide
256) 3-(5-ethyl-1,3-thiazol-2-yl)-5-[(2R)-tetrahydrofuran-2-ylmethoxy]-N-{(1R)-1-[2-(trifluoromethyl)pyrimidin-5-yl]ethyl}benzamide
257) 3-(5-cyclobutyl-1,3-thiazol-2-yl)-N-[(1R)-1-(6-methylpyridazin-3-yl)ethyl]-5-[(2R)-tetrahydrofuran-2-ylmethoxy]benzamide
258) 3-(5-cyclobutyl-1,3-thiazol-2-yl)-N-[(1R)-1-(5-methylpyrazin-2-yl)ethyl]-5-[(2R)-tetrahydrofuran-2-ylmethoxy]benzamide
259) 3-(5-cyclobutyl-1,3-thiazol-2-yl)-5-[(2R)-tetrahydrofuran-2-ylmethoxy]-N-{(1R)-1-[2-(trifluoromethyl)pyrimidin-5-yl]ethyl}benzamide
260) N-[(1R)-1-(6-methylpyridazin-3-yl)ethyl]-3-[5-(propan-2-yl)-1,3-thiazol-2-yl]-5-[(2R)-tetrahydrofuran-2-ylmethoxy]benzamide
261) N-[(1R)-1-(5-methylpyrazin-2-yl)ethyl]-3-[5-(propan-2-yl)-1,3-thiazol-2-yl]-5-[(2R)-tetrahydrofuran-2-ylmethoxy]benzamide
262) 3-[5-(propan-2-yl)-1,3-thiazol-2-yl]-5-[(2R)-tetrahydrofuran-2-ylmethoxy]-N-{(1R)-1-[2-(trifluoromethyl)pyrimidin-5-yl]ethyl}benzamide
263) 3-(5-ethyl-1,3-thiazol-2-yl)-N-[(1R)-1-(6-methylpyridazin-3-yl)ethyl]-5-[(3S)-tetrahydrofuran-3-ylmethoxy]benzamide
264) 3-(5-ethyl-1,3-thiazol-2-yl)-N-[(1R)-1-(5-methylpyrazin-2-yl)ethyl]-5-[(3S)-tetrahydrofuran-3-ylmethoxy]benzamide
265) 3-(5-ethyl-1,3-thiazol-2-yl)-5-[(3S)-tetrahydrofuran-3-ylmethoxy]-N-{(1R)-1-[2-(trifluoromethyl)pyrimidin-5-yl]ethyl}benzamide
266) N-[(1R)-1-(6-methylpyridazin-3-yl)ethyl]-3-[5-(propan-2-yl)-1,3-thiazol-2-yl]-5-[(3S)-tetrahydrofuran-3-ylmethoxy]benzamide
267) N-[(1R)-1-(5-methylpyrazin-2-yl)ethyl]-3-[5-(propan-2-yl)-1,3-thiazol-2-yl]-5-[(3S)-tetrahydrofuran-3-ylmethoxy]benzamide
268) 3-[5-(propan-2-yl)-1,3-thiazol-2-yl]-5-[(3S)-tetrahydrofuran-3-ylmethoxy]-N-{(1R)-1-[2-(trifluoromethyl)pyrimidin-5-yl]ethyl}benzamide
269) 3-(5-ethyl-1,3-thiazol-2-yl)-N-[(1R)-1-(6-methylpyridazin-3-yl)ethyl]-5-[(2S)-tetrahydrofuran-2-ylmethoxy]benzamide
270) 3-(5-ethyl-1,3-thiazol-2-yl)-N-[(1R)-1-(5-methylpyrazin-2-yl)ethyl]-5-[(2S)-tetrahydrofuran-2-ylmethoxy]benzamide
271) 3-(5-ethyl-1,3-thiazol-2-yl)-5-[(2S)-tetrahydrofuran-2-ylmethoxy]-N-{(1R)-1-[2-(trifluoromethyl)pyrimidin-5-yl]ethyl}benzamide
272) 3-(5-cyclobutyl-1,3-thiazol-2-yl)-N-[(1R)-1-(6-methylpyridazin-3-yl)ethyl]-5-[(2S)-tetrahydrofuran-2-ylmethoxy]benzamide
273) 3-(5-cyclobutyl-1,3-thiazol-2-yl)-N-[(1R)-1-(5-methylpyrazin-2-yl)ethyl]-5-[(2S)-tetrahydrofuran-2-ylmethoxy]benzamide
274) 3-(5-cyclobutyl-1,3-thiazol-2-yl)-5-[(2S)-tetrahydrofuran-2-ylmethoxy]-N-{(1R)-1-[2-(trifluoromethyl)pyrimidin-5-yl]ethyl}benzamide
275) N-[(1R)-1-(6-methylpyridazin-3-yl)ethyl]-3-[5-(propan-2-yl)-1,3-thiazol-2-yl]-5-[(2S)-tetrahydrofuran-2-ylmethoxy]benzamide
276) N-[(1R)-1-(5-methylpyrazin-2-yl)ethyl]-3-[5-(propan-2-yl)-1,3-thiazol-2-yl]-5-[(2S)-tetrahydrofuran-2-ylmethoxy]benzamide
277) 3-[5-(propan-2-yl)-1,3-thiazol-2-yl]-5-[(2S)-tetrahydrofuran-2-ylmethoxy]-N-{(1R)-1-[2-(trifluoromethyl)pyrimidin-5-yl]ethyl}benzamide
278) 3-(5-methyl-1,3-thiazol-2-yl)-5-[(3R)-tetrahydrofuran-3-yloxy]-N-{(1S)-1-[2-(trifluoromethyl)pyrimidin-5-yl]ethyl}benzamide
279) 3-(5-ethyl-1,3-thiazol-2-yl)-5-[(3R)-tetrahydrofuran-3-ylmethoxy]-N-{(1R)-1-[6-(trifluoromethyl)pyridazin-3-yl]ethyl}benzamide
280) 3-[5-(propan-2-yl)-1,3-thiazol-2-yl]-5-[(3R)-tetrahydrofuran-3-ylmethoxy]-N-{(1R)-1-[6-(trifluoromethyl)pyridazin-3-yl]ethyl}benzamide
281) 3-[5-(propan-2-yl)-1,3-thiazol-2-yl]-5-[(3R)-tetrahydrofuran-3-yloxy]-N-{(1R)-1-[6-(trifluoromethyl)pyridazin-3-yl]ethyl}benzamide
282) 3-(5-cyclobutyl-1,3-thiazol-2-yl)-5-[(3S)-tetrahydrofuran-3-yloxy]-N-{(1R)-1-[6-(trifluoromethyl)pyridazin-3-yl]ethyl}benzamide
283) 3-(5-ethyl-1,3-thiazol-2-yl)-5-[(3S)-tetrahydrofuran-3-ylmethoxy]-N-{(1R)-1-[6-(trifluoromethyl)pyridazin-3-yl]ethyl}benzamide
284) 3-(5-cyclobutyl-1,3-thiazol-2-yl)-5-[(3R)-tetrahydrofuran-3-ylmethoxy]-N-{(1R)-1-[6-(trifluoromethyl)pyridazin-3-yl]ethyl}benzamide
285) 3-[5-(propan-2-yl)-1,3-thiazol-2-yl]-5-[(3S)-tetrahydrofuran-3-ylmethoxy]-N-{(1R)-1-[6-(trifluoromethyl)pyridazin-3-yl]ethyl}benzamide
286) 3-[5-(propan-2-yl)-1,3-thiazol-2-yl]-5-[(2R)-tetrahydrofuran-2-ylmethoxy]-N-{(1R)-1-[6-(trifluoromethyl)pyridazin-3-yl]ethyl}benzamide
287) 3-(5-cyclobutyl-1,3-thiazol-2-yl)-5-[(2S)-tetrahydrofuran-2-ylmethoxy]-N-{(1R)-1-[6-(trifluoromethyl)pyridazin-3-yl]ethyl}benzamide
288) 3-[5-(propan-2-yl)-1,3-thiazol-2-yl]-5-[(2S)-tetrahydrofuran-2-ylmethoxy]-N-{(1R)-1-[6-(trifluoromethyl)pyridazin-3-yl]ethyl}benzamide
289) 3-(5-ethyl-1,3-thiazol-2-yl)-5-[(2S)-tetrahydrofuran-2-ylmethoxy]-N-{(1R)-1-[6-(trifluoromethyl)pyridazin-3-yl]ethyl}benzamide
290) 3-(5-methyl-1,3-thiazol-2-yl)-5-{[1-(2,2,2-trifluoroethyl)piperidin-4-yl]oxy}-N-{(1R)-1-[2-(trifluoromethyl)pyrimidin-5-yl]ethyl}benzamide
291) 3-{[1-(2,2-difluoroethyl)piperidin-4-yl]oxy}-5-(5-methyl-1,3-thiazol-2-yl)-N-{(1R)-1-[2-(trifluoromethyl)pyrimidin-5-yl]ethyl}benzamide
292) 3-{[1-(2,2-difluoroethyl)piperidin-4-yl]oxy}-N-[(1R)-1-(6-methylpyridazin-3-yl)ethyl]-5-(5-methyl-1,3-thiazol-2-yl)benzamide
293) 3-{[1-(2,2-difluoroethyl)piperidin-4-yl]oxy}-N-[(1R)-1-(5-methylpyrazin-2-yl)ethyl]-5-(5-methyl-1,3-thiazol-2-yl)benzamide
294) 3-{[1-(2,2-difluoroethyl)piperidin-4-yl]oxy}-N-[(6-methylpyridazin-3-yl)methyl]-5-(5-methyl-1,3-thiazol-2-yl)benzamide
295) 3-{[1-(2,2-difluoroethyl)piperidin-4-yl]oxy}-5-(5-methyl-1,3-thiazol-2-yl)-N-{(1S)-1-[2-(trifluoromethyl)pyrimidin-5-yl]ethyl}benzamide
296) 3-{[1-(2,2-difluoroethyl)piperidin-4-yl]oxy}-N-[(1R)-1-(2-methylpyrimidin-5-yl)ethyl]-5-(5-methyl-1,3-thiazol-2-yl)benzamide
297) 3-(5-methyl-1,3-thiazol-2-yl)-5-{[1-(2,2,2-trifluoroethyl)piperidin-4-yl]oxy}-N-{(1S)-1-[2-(trifluoromethyl)pyrimidin-5-yl]ethyl}benzamide
298) N-[(1R)-1-(6-methylpyridazin-3-yl)ethyl]-3-(5-methyl-1,3-thiazol-2-yl)-5-{[1-(2,2,2-trifluoroethyl)piperidin-4-yl]oxy}benzamide
299) 3-(5-methyl-1,3-thiazol-2-yl)-5-{[1-(2,2,2-trifluoroethyl)piperidin-4-yl]oxy}-N-{(1R)-1-[6-(trifluoromethyl)pyridazin-3-yl]ethyl}benzamide
300) N-[(1R)-1-(5-methylpyrazin-2-yl)ethyl]-3-(5-methyl-1,3-thiazol-2-yl)-5-{[1-(2,2,2-trifluoroethyl)piperidin-4-yl]oxy}benzamide
301) N-[(6-methylpyridazin-3-yl)methyl]-3-(5-methyl-1,3-thiazol-2-yl)-5-{[1-(2,2,2-trifluoroethyl)piperidin-4-yl]oxy}benzamide
302) N-[(1R)-1-(2-methylpyrimidin-5-yl)ethyl]-3-(5-methyl-1,3-thiazol-2-yl)-5-{[1-(2,2,2-trifluoroethyl)piperidin-4-yl]oxy}benzamide
303) 3-(5-chloro-1,3-thiazol-2-yl)-N-[(1R)-1-(6-methylpyridazin-3-yl)ethyl]-5-[(3S)-tetrahydrofuran-3-ylmethoxy]benzamide
304) 3-(5-chloro-1,3-thiazol-2-yl)-N-[(1R)-1-(6-methylpyridazin-3-yl)ethyl]-5-[(3R)-tetrahydrofuran-3-yloxy]benzamide
305) 3-(5-chloro-1,3-thiazol-2-yl)-5-[(3R)-tetrahydrofuran-3-yloxy]-N-{(1R)-1-[6-(trifluoromethyl)pyridazin-3-yl]ethyl}benzamide
306) 3-(5-chloro-1,3-thiazol-2-yl)-N-[(1R)-1-(5-methylpyrazin-2-yl)ethyl]-5-[(3R)-tetrahydrofuran-3-yloxy]benzamide
307) 3-(5-chloro-1,3-thiazol-2-yl)-5-[(3S)-tetrahydrofuran-3-yloxy]-N-{(1R)-1-[6-(trifluoromethyl)pyridazin-3-yl]ethyl}benzamide
308) 3-(5-chloro-1,3-thiazol-2-yl)-N-[(1R)-1-(5-methylpyrazin-2-yl)ethyl]-5-[(3S)-tetrahydrofuran-3-yloxy]benzamide
309) 3-(5-chloro-1,3-thiazol-2-yl)-N-[(1R)-1-(6-methylpyridazin-3-yl)ethyl]-5-[(3S)-tetrahydrofuran-3-yloxy]benzamide
310) 3-(5-chloro-1,3-thiazol-2-yl)-5-[(3S)-tetrahydrofuran-3-ylmethoxy]-N-{(1R)-1-[2-(trifluoromethyl)pyrimidin-5-yl]ethyl}benzamide
311) 3-(5-chloro-1,3-thiazol-2-yl)-N-[(1R)-1-(5-methylpyrazin-2-yl)ethyl]-5-(tetrahydro-2H-pyran-4-yloxy)benzamide
312) 3-(5-chloro-1,3-thiazol-2-yl)-N-[(1R)-1-(6-methylpyridazin-3-yl)ethyl]-5-(tetrahydro-2H-pyran-4-yloxy)benzamide
313) 3-(5-chloro-1,3-thiazol-2-yl)-5-(tetrahydro-2H-pyran-4-yloxy)-N-{(1R)-1-[6-(trifluoromethyl)pyridazin-3-yl]ethyl}benzamide
314) 3-[(3-methyloxetan-3-yl)methoxy]-5-(5-methyl-1,3-thiazol-2-yl)-N-{(1R)-1-[2-(trifluoromethyl)pyrimidin-5-yl]ethyl}benzamide
315) 3-(2-hydroxy-2-methylpropoxy)-5-(5-methyl-1,3-thiazol-2-yl)-N-{(1R)-1-[2-(trifluoromethyl)pyrimidin-5-yl]ethyl}benzamide
316) 3-[(2-methyltetrahydrofuran-2-yl)methoxy]-5-(5-methyl-1,3-thiazol-2-yl)-N-{(1R)-1-[2-(trifluoromethyl)pyrimidin-5-yl]ethyl}benzamide, as a mixture of two diastereoisomers
317) Diastereoisomer 1; 3-[(2-methyltetrahydrofuran-2-yl)methoxy]-5-(5-methyl-1,3-thiazol-2-yl)-N-{(1R)-1-[2-(trifluoromethyl)pyrimidin-5-yl]ethyl}benzamide
318) Diastereoisomer 2; 3-[(2-methyltetrahydrofuran-2-yl)methoxy]-5-(5-methyl-1,3-thiazol-2-yl)-N-{(1R)-1-[2-(trifluoromethyl)pyrimidin-5-yl]ethyl}benzamide
319) 3-[(3-methyltetrahydrofuran-3-yl)methoxy]-5-(5-methyl-1,3-thiazol-2-yl)-N-{(1R)-1-[2-(trifluoromethyl)pyrimidin-5-yl]ethyl}benzamide, as a mixture of two diastereoisomers
320) Diastereoisomer 1; 3-[(3-methyltetrahydrofuran-3-yl)methoxy]-5-(5-methyl-1,3-thiazol-2-yl)-N-{(1R)-1-[2-(trifluoromethyl)pyrimidin-5-yl]ethyl}benzamide
321) Diastereoisomer 2; 3-[(3-methyltetrahydrofuran-3-yl)methoxy]-5-(5-methyl-1,3-thiazol-2-yl)-N-{(1R)-1-[2-(trifluoromethyl)pyrimidin-5-yl]ethyl}benzamide
322) 3-[(1-methyl-6-oxopiperidin-3-yl)oxy]-5-(5-methyl-1,3-thiazol-2-yl)-N-{(1R)-1-[2-(trifluoromethyl)pyrimidin-5-yl]ethyl}benzamide, as a mixture of two diastereoisomers
323) Diastereoisomer 1; 3-[(1-methyl-6-oxopiperidin-3-yl)oxy]-5-(5-methyl-1,3-thiazol-2-yl)-N-{(1R)-1-[2-(trifluoromethyl)pyrimidin-5-yl]ethyl}benzamide
324) Diastereoisomer 2; 3-[(1-methyl-6-oxopiperidin-3-yl)oxy]-5-(5-methyl-1,3-thiazol-2-yl)-N-{(1R)-1-[2-(trifluoromethyl)pyrimidin-5-yl]ethyl}benzamide
325) 3-[(3-hydroxybutan-2-yl)oxy]-5-(5-methyl-1,3-thiazol-2-yl)-N-{(1R)-1-[2-(trifluoromethyl)pyrimidin-5-yl]ethyl}benzamide, as a mixture of cis isomers
326) Cis Isomer 1; 3-[(3-hydroxybutan-2-yl)oxy]-5-(5-methyl-1,3-thiazol-2-yl)-N-{(1R)-1-[2-(trifluoromethyl)pyrimidin-5-yl]ethyl}benzamide
327) Cis Isomer 2; 3-[(3-hydroxybutan-2-yl)oxy]-5-(5-methyl-1,3-thiazol-2-yl)-N-{(1R)-1-[2-(trifluoromethyl)pyrimidin-5-yl]ethyl}benzamide
328) 3-[(7-methyl-3-oxa-7-azabicyclo[3.3.1]non-9-yl)oxy]-5-(5-methyl-1,3-thiazol-2-yl)-N-{(1R)-1-[2-(trifluoromethyl)pyrimidin-5-yl]ethyl}benzamide, as a mixture of two stereoisomers
329) Stereoisomer 1; 3-[(7-methyl-3-oxa-7-azabicyclo[3.3.1]non-9-yl)oxy]-5-(5-methyl-1,3-thiazol-2-yl)-N-{(1R)-1-[2-(trifluoromethyl)pyrimidin-5-yl]ethyl}benzamide
330) Stereoisomer 2; 3-[(7-methyl-3-oxa-7-azabicyclo[3.3.1]non-9-yl)oxy]-5-(5-methyl-1,3-thiazol-2-yl)-N-{(1R)-1-[2-(trifluoromethyl)pyrimidin-5-yl]ethyl}benzamide
331) 3-[(7-isopropyl-3-oxa-7-azabicyclo[3.3.1]non-9-yl)oxy]-5-(5-methyl-1,3-thiazol-2-yl)-N-{(1R)-1-[2-(trifluoromethyl)pyrimidin-5-yl]ethyl}benzamide, as a mixture of two stereoisomers
332) methyl 9-[3-(5-methyl-1,3-thiazol-2-yl)-5-({(1R)-1-[2-(trifluoromethyl)pyrimidin-5-yl]ethyl}carbamoyl)phenoxy]-3-oxa-7-azabicyclo[3.3.1]nonane-7-carboxylate, as a mixture of two stereoisomers
333) tert-buty (2R)-2-{[3-(5-methyl-1,3-thiazol-2-yl)-5-({(1R)-1-[2-(trifluoromethyl)pyrimidin-5-yl]ethyl}carbamoyl)phenoxy]methyl}morpholine-4-carboxylate
334) 3-(5-methyl-1,3-thiazol-2-yl)-5-[(2R)-morpholin-2-ylmethoxy]-N-{(1R)-1-[2-(trifluoromethyl)pyrimidin-5-yl]ethyl}benzamide
335) 3-{[(2R)-4-methylmorpholin-2-yl]methoxy}-5-(5-methyl-1,3-thiazol-2-yl)-N-{(1R)-1-[2-(trifluoromethyl)pyrimidin-5-yl]ethyl}benzamide
336) tert-butyl (2S)-2-{[3-(5-methyl-1,3-thiazol-2-yl)-5-({(1R)-1-[2-(trifluoromethyl)pyrimidin-5-yl]ethyl}carbamoyl)phenoxy]methyl}morpholine-4-carboxylate
337) 3-(5-methyl-1,3-thiazol-2-yl)-5-[(2S)-morpholin-2-ylmethoxy]-N-{(1R)-1-[2-(trifluoromethyl)pyrimidin-5-yl]ethyl}benzamide
338) 3-{[(2S)-4-methylmorpholin-2-yl]methoxy}-5-(5-methyl-1,3-thiazol-2-yl)-N-{(1R)-1-[2-(trifluoromethyl)pyrimidin-5-yl]ethyl}benzamide
339) 3-(5-methyl-1,3-thiazol-2-yl)-5-[morpholin-2-ylmethoxy]-N-{(1R)-1-[2-(trifluoromethyl)pyrimidin-5-yl]ethyl}benzamide, as a mixture of diastereoisomers
340) 3-{[4-methylmorpholin-2-yl]methoxy}-5-(5-methyl-1,3-thiazol-2-yl)-N-{(1R)-1-[2-(trifluoromethyl)pyrimidin-5-yl]ethyl}benzamide, as a mixture of diastereoisomers
341) Diastereoisomer 1; 3-(fluoropiperidin-3-yl)methoxy}-5-(5-methyl-1,3-thiazol-2-yl)-N-{(1R)-1-[2-(trifluoromethyl)pyrimidin-5-yl]ethyl}benzamide
342) Diastereoisomer 2; 3-(fluoropiperidin-3-yl)methoxy}-5-(5-methyl-1,3-thiazol-2-yl)-N-{(1R)-1-[2-(trifluoromethyl)pyrimidin-5-yl]ethyl}benzamide
343) Diastereoisomer 1; 3-{[3-fluoro-1-methylpiperidin-3-yl]methoxy}-5-(5-methyl-1,3-thiazol-2-yl)-N-{(1R)-1-[2-(trifluoromethyl)pyrimidin-5-yl]ethyl}benzamide
344) Diastereoisomer 2; 3-{[3-fluoro-1-methylpiperidin-3-yl]methoxy}-5-(5-methyl-1,3-thiazol-2-yl)-N-{(1R)-1-[2-(trifluoromethyl)pyrimidin-5-yl]ethyl}benzamide
345) 3-[(3-fluoroazetidin-3-yl)methoxy]-5-(5-methyl-1,3-thiazol-2-yl)-N-{(1R)-1-[2-(trifluoromethyl)pyrimidin-5-yl]ethyl}benzamide
346) 3-{[4,4-difluoropiperidin-3-yl]methoxy}-5-(5-methyl-1,3-thiazol-2-yl)-N-{(1R)-1-[2-(trifluoromethyl)pyrimidin-5-yl]ethyl}benzamide, as a mixture of 2 diastereoisomers
347) 3-{[(3R)-4-methylmorpholin-3-yl]methoxy}-5-(5-methyl-1,3-thiazol-2-yl)-N-{(1R)-1-[2-(trifluoromethyl)pyrimidin-5-yl]ethyl}benzamide
348) 3-{[(3S)-4-methylmorpholin-3-yl]methoxy}-5-(5-methyl-1,3-thiazol-2-yl)-N-{(1R)-1-[6-(trifluoromethyl)pyridin-3-yl]ethyl}benzamide
349) 3-{[(3S)-4-methylmorpholin-3-yl]methoxy}-5-(5-methyl-1,3-thiazol-2-yl)-N-{(1R)-1-[2-(trifluoromethyl)pyrimidin-5-yl]ethyl}benzamide
350) 3-{[(3R)-4-methylmorpholin-3-yl]methoxy}-5-(5-methyl-1,3-thiazol-2-yl)-N-{(1R)-1-[6-(trifluoromethyl)pyridin-3-yl]ethyl}benzamide
351) 3-{[4-fluoro-1-methylpyrrolidin-2-yl]methoxy}-5-(5-methyl-1,3-thiazol-2-yl)-N-{(1R)-1-[2-(trifluoromethyl)pyrimidin-5-yl]ethyl}benzamide, as a mixture of stereoisomers
352) 3-{[4-fluoro-1-methylpyrrolidin-2-yl]methoxy}-5-(5-methyl-1,3-thiazol-2-yl)-N-{(1R)-1-[6-(trifluoromethyl)pyridin-3-yl]ethyl}benzamide, as a mixture of stereoisomers
353) 3-{[(2R)-4-methylmorpholin-2-yl]methoxy}-5-(5-methyl-1,3-thiazol-2-yl)-N-{(1R)-1-[6-(trifluoromethyl)pyridin-3-yl]ethyl}benzamide
354) 3-(5-chloro-1,3-thiazol-2-yl)-5-{[(2R)-4-methylmorpholin-2-yl]methoxy}-N-{(1R)-1-[2-(trifluoromethyl)pyrimidin-5-yl]ethyl}benzamide
355) 3-{[(2S)-4-methylmorpholin-2-yl]methoxy}-5-(5-methyl-1,3-thiazol-2-yl)-N-{[2-(trifluoromethyl)pyrimidin-5-yl]methyl}benzamide
356) N-{(1R)-1-[6-(difluoromethyl)pyridin-3-yl]ethyl}-3-{[(2R)-4-methylmorpholin-2-yl]methoxy}-5-(5-methyl-1,3-thiazol-2-yl)benzamide
357) 3-{[(2S)-4-methylmorpholin-2-yl]methoxy}-5-(5-methyl-1,3-thiazol-2-yl)-N-{(1R)-1-[6-(trifluoromethyl)pyridin-3-yl]ethyl}benzamide
358) 3-[(3-fluoro-1-methylazetidin-3-yl)methoxy]-5-(5-methyl-1,3-thiazol-2-yl)-N-{(1R)-1-[2-(trifluoromethyl)pyrimidin-5-yl]ethyl}benzamide
359) 3-{[(2R)-4-methylmorpholin-2-yl]methoxy}-5-(5-methyl-1,3-thiazol-2-yl)-N-{[2-(trifluoromethyl)pyrimidin-5-yl]methyl}benzamide
360) 3-{[(2R)-4-methylmorpholin-2-yl]methoxy}-5-(5-methyl-1,3-thiazol-2-yl)-N-{(1R)-1-[6-(trifluoromethyl)pyridazin-3-yl]ethyl}benzamide
361) 3-{[(2S)-4-methylmorpholin-2-yl]methoxy}-5-(5-methyl-1,3-thiazol-2-yl)-N-{(1R)-1-[6-(trifluoromethyl)pyridazin-3-yl]ethyl}benzamide
362) 3-(5-ethyl-1,3-thiazol-2-yl)-5-{[(2S)-4-methylmorpholin-2-yl]methoxy}-N-{(1R)-1-[2-(trifluoromethyl)pyrimidin-5-yl]ethyl}benzamide
363) 3-(5-chloro-1,3-thiazol-2-yl)-5-{[(2S)-4-methylmorpholin-2-yl]methoxy}-N-{(1R)-1-[2-(trifluoromethyl)pyrimidin-5-yl]ethyl}benzamide
364) 3-(5-ethyl-1,3-thiazol-2-yl)-5-{[(2R)-4-methylmorpholin-2-yl]methoxy}-N-{(1R)-1-[2-(trifluoromethyl)pyrimidin-5-yl]ethyl}benzamide
365) 3-{[(2S)-1-methylpyrrolidin-2-yl]methoxy}-5-(5-methyl-1,3-thiazol-2-yl)-N-{(1R)-1-[2-(trifluoromethyl)pyrimidin-5-yl]ethyl}benzamide
366) 3-{[(2R)-1-methylpyrrolidin-2-yl]methoxy}-5-(5-methyl-1,3-thiazol-2-yl)-N-{(1R)-1-[2-(trifluoromethyl)pyrimidin-5-yl]ethyl}benzamide
367) 3-[(1-methylpiperidin-4-yl)methoxy]-5-(5-methyl-1,3-thiazol-2-yl)-N-{(1R)-1-[2-(trifluoromethyl)pyrimidin-5-yl]ethyl}benzamide
368) 3-(5-methyl-1,3-thiazol-2-yl)-5-{[(2R)-4-(propan-2-yl)morpholin-2-yl]methoxy}-N-{(1R)-1-[2-(trifluoromethyl)pyrimidin-5-yl]ethyl}benzamide
369) 3-(5-methyl-1,3-thiazol-2-yl)-5-{[(2S)-4-(propan-2-yl)morpholin-2-yl]methoxy}-N-{(1R)-1-[2-(trifluoromethyl)pyrimidin-5-yl]ethyl}benzamide
370) 3-{[4,4-difluoro-1-methylpiperidin-3-yl]methoxy}-5-(5-methyl-1,3-thiazol-2-yl)-N-{(1R)-1-[2-(trifluoromethyl)pyrimidin-5-yl]ethyl}benzamide, as a mixture of 2 diastereoisomers
371) Diastereoisomer 1; 3-{[4,4-difluoro-1-methylpiperidin-3-yl]methoxy}-5-(5-methyl-1,3-thiazol-2-yl)-N-{(1R)-1-[2-(trifluoromethyl)pyrimidin-5-yl]ethyl}benzamide
372) Diastereoisomer 2; 3-{[4,4-difluoro-1-methylpiperidin-3-yl]methoxy}-5-(5-methyl-1,3-thiazol-2-yl)-N-{(1R)-1-[2-(trifluoromethyl)pyrimidin-5-yl]ethyl}benzamide
373) 3-[(3-fluoro-1-methylazetidin-3-yl)methoxy]-5-(5-methyl-1,3-thiazol-2-yl)-N-{(1R)-1-[6-(trifluoromethyl)pyridin-3-yl]ethyl}benzamide
374) 3-(5-ethyl-1,3-thiazol-2-yl)-5-[(3-fluoro-1-methylazetidin-3-yl)methoxy]-N-{(1R)-1-[6-(trifluoromethyl)pyridin-3-yl]ethyl}benzamide
375) 3-{[(3R)-4-methylmorpholin-3-yl]methoxy}-5-(5-methyl-1,3-thiazol-2-yl)-N-{(1R)-1-[6-(trifluoromethyl)pyridazin-3-yl]ethyl}benzamide
376) 3-{[(3S)-4-methylmorpholin-3-yl]methoxy}-5-(5-methyl-1,3-thiazol-2-yl)-N-{(1R)-1-[6-(trifluoromethyl)pyridazin-3-yl]ethyl}benzamide
377) 3-{[(2R)-4-ethylmorpholin-2-yl]methoxy}-5-(5-methyl-1,3-thiazol-2-yl)-N-{(1R)-1-[2-(trifluoromethyl)pyrimidin-5-yl]ethyl}benzamide
378) 3-{[(2R)-4-(2,2-difluoroethyl)morpholin-2-yl]methoxy}-5-(5-methyl-1,3-thiazol-2-yl)-N-{(1R)-1-[2-(trifluoromethyl)pyrimidin-5-yl]ethyl}benzamide
379) methyl (2R)-2-{[3-(5-methyl-1,3-thiazol-2-yl)-5-({(1R)-1-[2-(trifluoromethyl)pyrimidin-5-yl]ethyl}carbamoyl)phenoxy]methyl}morpholine-4-carboxylate
380) methyl (2S)-2-{[3-(5-methyl-1,3-thiazol-2-yl)-5-({(1R)-1-[2-(trifluoromethyl)pyrimidin-5-yl]ethyl}carbamoyl)phenoxy]methyl}morpholine-4-carboxylate
381) 3-(azetidin-3-ylmethoxy)-5-(5-methyl-1,3-thiazol-2-yl)-N-{(1R)-1-[2-(trifluoromethyl)pyrimidin-5-yl]ethyl}benzamide
382) 3-{[(3R)-4-methyl-5-oxomorpholin-3-yl]methoxy}-5-(5-methyl-1,3-thiazol-2-yl)-N-{(1R)-1-[2-(trifluoromethyl)pyrimidin-5-yl]ethyl}benzamide
383) 3-(5-methyl-1,3-thiazol-2-yl)-5-{[(3R)-5-oxomorpholin-3-yl]methoxy}-N-{(1R)-1-[2-(trifluoromethyl)pyrimidin-5-yl]ethyl}benzamide
384) 3-{[(5S)-3-methyl-2-oxo-1,3-oxazolidin-5-yl]methoxy}-5-(5-methyl-1,3-thiazol-2-yl)-N-{(1R)-1-[2-(trifluoromethyl)pyrimidin-5-yl]ethyl}benzamide
385) 3-{[(5R)-3-methyl-2-oxo-1,3-oxazolidin-5-yl]methoxy}-5-(5-methyl-1,3-thiazol-2-yl)-N-{(1R)-1-[2-(trifluoromethyl)pyrimidin-5-yl]ethyl}benzamide
386) 3-{[(2R)-4-methyl-5-oxomorpholin-2-yl]methoxy}-5-(5-methyl-1,3-thiazol-2-yl)-N-{(1R)-1-[2-(trifluoromethyl)pyrimidin-5-yl]ethyl}benzamide
387) 3-(5-methyl-1,3-thiazol-2-yl)-5-{[(2S)-5-oxomorpholin-2-yl]methoxy}-N-{(1R)-1-[2-(trifluoromethyl)pyrimidin-5-yl]ethyl}benzamide
388) 3-{[(2S)-4-methyl-5-oxomorpholin-2-yl]methoxy}-5-(5-methyl-1,3-thiazol-2-yl)-N-{(1R)-1-[2-(trifluoromethyl)pyrimidin-5-yl]ethyl}benzamide
389) 3-{[(3S)-4-methyl-5-oxomorpholin-3-yl]methoxy}-5-(5-methyl-1,3-thiazol-2-yl)-N-{(1R)-1-[2-(trifluoromethyl)pyrimidin-5-yl]ethyl}benzamide
390) 3-(5-methyl-1,3-thiazol-2-yl)-5-{[(3S)-5-oxomorpholin-3-yl]methoxy}-N-{(1R)-1-[2-(trifluoromethyl)pyrimidin-5-yl]ethyl}benzamide
391) tert-butyl 1-{[3-(5-methyl-1,3-thiazol-2-yl)-5-({(1R)-1-[2-(trifluoromethyl)pyrimidin-5-yl]ethyl}carbamoyl)phenoxy]methyl}-2-oxa-5-azabicyclo[2.2.1]heptane-5-carboxylate, as a mixture of 2 diastereoisomers
392) 3-[(5-isopropyl-2-oxa-5-azabicyclo[2.2.1]hept-1-yl)methoxy]-5-(5-methyl-1,3-thiazol-2-yl)-N-{(1R)-1-[2-(trifluoromethyl)pyrimidin-5-yl]ethyl}benzamide, as a mixture of 2 diastereoisomers
393) 3-[(5-methyl-2-oxa-5-azabicyclo[2.2.1]hept-1-yl)methoxy]-5-(5-methyl-1,3-thiazol-2-yl)-N-{(1R)-1-[2-(trifluoromethyl)pyrimidin-5-yl]ethyl}benzamide, as a mixture of 2 diastereoisomers
394) 3-(5-methyl-1,3-thiazol-2-yl)-5-[(1S,4S)-2-oxa-5-azabicyclo[2.2.1]hept-1-ylmethoxy]-N-{(1R)-1-[2-(trifluoromethyl)pyrimidin-5-yl]ethyl}benzamide, as a mixture of 2 diastereoisomers
395) 3-(5-methyl-1,3-thiazol-2-yl)-5-[(5-propyl-2-oxa-5-azabicyclo[2.2.1]hept-1-yl)methoxy]-N-{(1R)-1-[2-(trifluoromethyl)pyrimidin-5-yl]ethyl}benzamide, as a mixture of 2 diastereoisomers
396) methyl 1-{[3-(5-methyl-1,3-thiazol-2-yl)-5-({(1R)-1-[2-(trifluoromethyl)pyrimidin-5-yl]ethyl}carbamoyl)phenoxy]methyl}-2-oxa-5-azabicyclo[2.2.1]heptane-5-carboxylate, as a mixture of 2 diastereoisomers
397) ethyl 1-{[3-(5-methyl-1,3-thiazol-2-yl)-5-({(1R)-1-[2-(trifluoromethyl)pyrimidin-5-yl]ethyl}carbamoyl)phenoxy]methyl}-2-oxa-5-azabicyclo[2.2.1]heptane-5-carboxylate, as a mixture of 2 diastereoisomers
398) 3-{[(2S)-4-ethylmorpholin-2-yl]methoxy}-5-(5-methyl-1,3-thiazol-2-yl)-N-{(1R)-1-[2-(trifluoromethyl)pyrimidin-5-yl]ethyl}benzamide
399) tert-butyl (2R)-2-{[3-(5-methyl-1,3-thiazol-2-yl)-5-({(1S)-1-[2-(trifluoromethyl)pyrimidin-5-yl]ethyl}carbamoyl)phenoxy]methyl}morpholine-4-carboxylate
400) 3-(5-methyl-1,3-thiazol-2-yl)-5-[(2R)-morpholin-2-ylmethoxy]-N-{(1S)-1-[2-(trifluoromethyl)pyrimidin-5-yl]ethyl}benzamide
401) 3-(5-ethyl-1,3-thiazol-2-yl)-5-[(2S)-morpholin-2-ylmethoxy]-N-{(1R)-1-[6-(trifluoromethyl)pyridazin-3-yl]ethyl}benzamide
402) 3-(5-ethyl-1,3-thiazol-2-yl)-5-[(2R)-morpholin-2-ylmethoxy]-N-{(1R)-1-[6-(trifluoromethyl)pyridazin-3-yl]ethyl}benzamide
403) 3-{[(2R)-4-methylmorpholin-2-yl]methoxy}-5-(5-methyl-1,3-thiazol-2-yl)-N-{(1S)-1-[2-(trifluoromethyl)pyrimidin-5-yl]ethyl}benzamide
404) 3-{[(2S)-4-methylmorpholin-2-yl]methoxy}-5-(5-methyl-1,3-thiazol-2-yl)-N-{(1S)-1-[2-(trifluoromethyl)pyrimidin-5-yl]ethyl}benzamide
405) 3-(5-ethyl-1,3-thiazol-2-yl)-5-{[(2S)-4-methylmorpholin-2-yl]methoxy}-N-{(1R)-1-[6-(trifluoromethyl)pyridazin-3-yl]ethyl}benzamide
406) 3-(5-ethyl-1,3-thiazol-2-yl)-5-{[(2R)-4-methylmorpholin-2-yl]methoxy}-N-{(1R)-1-[6-(trifluoromethyl)pyridazin-3-yl]ethyl}benzamide.

Also disclosed is the use of following compounds for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma,
3-(5-Methyl-1,3-thiazol-2-yl)-5-[(3R)-tetrahydrofuran-3-yloxy]-N-{(1R)-1-[2-(trifluoromethyl)pyrimidin-5-yl]ethyl}benzamide;
3-(5-Methyl-1,3-thiazol-2-yl)-5-[(3S)-tetrahydrofuran-3-yloxy]-N-{(1R)-1-[2-(trifluoromethyl)pyrimidin-5-yl]ethyl}benzamide;
3-(5-Methyl-1,3-thiazol-2-yl)-5-(oxetan-3-yloxy)-N-{(1R)-1-[2-(trifluoromethyl)pyrimidin-5-yl]ethyl}benzamide;
3-(5-Ethyl-1,3-thiazol-2-yl)-5-[(3R)-tetrahydrofuran-3-yloxy]-N-{(1R)-1-[2-(trifluoromethyl)pyrimidin-5-yl]ethyl}benzamide;
3-(5-Ethyl-1,3-thiazol-2-yl)-5-[(3S)-tetrahydrofuran-3-yloxy]-N-{(1R)-1-[2-(trifluoromethyl)pyrimidin-5-yl]ethyl}benzamide;
3-(5-Ethyl-1,3-thiazol-2-yl)-5-(oxetan-3-yloxy)-N-{(1R)-1-[2-(trifluoromethyl)pyrimidin-5-yl]ethyl}benzamide;
3-(5-Methyl-1,3-thiazol-2-yl)-5-[(3R)-tetrahydrofuran-3-yloxy]-N-{(1R)-1-[6-(trifluoromethyl)pyridazin-3-yl]ethyl}benzamide;
3-(5-Methyl-1,3-thiazol-2-yl)-5-[(3S)-tetrahydrofuran-3-yloxy]-N-{(1R)-1-[6-(trifluoromethyl)pyridazin-3-yl]ethyl}benzamide.

Preferred embodiment of the present invention is the use of following compounds for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma, namely
3-(5-methyl-1,3-thiazol-2-yl)-5-[(3R)-tetrahydrofuran-3-yloxy]-N-{(1R)-1-[2-(trifluoromethyl)pyrimidin-5-yl]ethyl}benzamide;
3-(5-methyl-1,3-thiazol-2-yl)-5-[(3S)-tetrahydrofuran-3-yloxy]-N-{(1R)-1-[2-(trifluoromethyl)pyrimidin-5-yl]ethyl}benzamide.

An even more preferred embodiment of the present invention is the use of 3-(5-methyl-1,3-thiazol-2-yl)-5-[(3R)-tetrahydrofuran-3-yloxy]-N-{(1R)-1-[2-(trifluoro-methyl)pyrimidin-5-yl]ethyl}benzamide for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the treatment or prophylaxis of Chronic Cough (CC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the treatment or prophylaxis of Refractory or Unexplained Chronic Cough (RUCC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the treatment or prophylaxis of Idiopathic Chronic Cough (ICC) also known as Unexplained Chronic Cough (UCC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the treatment or prophylaxis of Refractory Chronic Cough (RCC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the oral treatment or oral prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the oral treatment or oral prophylaxis of Chronic Cough (CC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the oral treatment or oral prophylaxis of Refractory or Unexplained Chronic Cough (RUCC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the oral treatment or oral prophylaxis of Idiopathic Chronic Cough (ICC) also known as Unexplained Chronic Cough (UCC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the oral treatment or oral prophylaxis of Refractory Chronic Cough (RCC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the long-term treatment of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the long-term treatment of Chronic Cough (CC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the long-term treatment of Refractory or Unexplained Chronic Cough (RUCC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the long-term treatment of Idiopathic Chronic Cough (ICC) also known as Unexplained Chronic Cough (UCC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the long-term treatment of Refractory Chronic Cough (RCC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the oral and long-term treatment of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the oral and long-term treatment of Chronic Cough (CC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the oral and long-term treatment of Refractory or Unexplained Chronic Cough (RUCC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the oral and long-term treatment of Idiopathic Chronic Cough (ICC) also known as Unexplained Chronic Cough (UCC).

Another preferred embodiment of the invention relates to the use of 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide for the oral and long-term treatment of Refractory Chronic Cough (RCC).

Another preferred embodiment of the present invention is the use of the following compounds, namely
3-(5-ethyl-1,3-thiazol-2-yl)-5-{[(2R)-4-methylmorpholin-2-yl]methoxy}-N-{(1R)-1-[2-(trifluoromethyl)pyrimidin-5-yl]ethyl}benzamide;
3-{[(2R)-4-methylmorpholin-2-yl]methoxy}-5-(5-methyl-1,3-thiazol-2-yl)-N-{(1R)-1-[2-(trifluoromethyl)pyrimidin-5-yl]ethyl}benzamide;
3-(5-ethyl-1,3-thiazol-2-yl)-5-{[(2R)-4-methylmorpholin-2-yl]methoxy}-N-{(1R)-1-[6-(trifluoromethyl)pyridazin-3-yl]ethyl}benzamide
   for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

An even more preferred embodiment of the present invention is the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the treatment or prophylaxis of Chronic Cough (CC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the treatment or prophylaxis of Refractory or Unexplained Chronic Cough (RUCC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the treatment or prophylaxis of Idiopathic Chronic Cough (ICC) also known as Unexplained Chronic Cough (UCC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the treatment or prophylaxis of Refractory Chronic Cough (RCC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the oral treatment or oral prophylaxis of diseases or disorders which are associated with nerve fibre sensitization, in particular for the oral treatment and prophylaxis of Chronic Cough (CC), Idiopathic Pulmonary Fibrosis (IPF), Chronic Obstructive Pulmonary Disorder (COPD), and asthma.

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the oral treatment or oral prophylaxis of Chronic Cough (CC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the oral treatment or oral prophylaxis of Refractory or Unexplained Chronic Cough (RUCC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the oral treatment or oral prophylaxis of Idiopathic Chronic Cough (ICC) also known as Unexplained Chronic Cough (UCC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the oral treatment or oral prophylaxis of Refractory Chronic Cough (RCC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl), pyrimidin-5-yl]ethyl}benzamide for the long-term treatment of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the long-term treatment of Chronic Cough (CC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the long-term treatment of Refractory or Unexplained Chronic Cough (RUCC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the long-term treatment of Idiopathic Chronic Cough (ICC) also known as Unexplained Chronic Cough (UCC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the long-term treatment of Refractory Chronic Cough (RCC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the oral and long-term treatment of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the oral and long-term treatment of Chronic Cough (CC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the oral and long-term treatment of Refractory or Unexplained Chronic Cough (RUCC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the oral and long-term treatment of Idiopathic Chronic Cough (ICC) also known as Unexplained Chronic Cough (UCC).

Another preferred embodiment of the invention relates to the use of 3-{[(2R)-4-methylmorpholin-2-yl]methoxy}-5-(5-methyl-1,3-thiazol-2-yl)-N-{(1R)-1-[2-(tri-fluoromethyl)pyrimidin-5-yl]ethyl}benzamide for the oral and long-term treatment of Refractory Chronic Cough (RCC).

Another preferred embodiment of the present invention is the use of the following compounds, namely
Trans Isomer 2; 3-{[3-hydroxybutan-2-yl]oxy}-5-(5-methyl-1,3-thiazol-2-yl)-N-{(1R)-1-[2-(trifluoromethyl)pyrimidin-5-yl]ethyl}benzamide;
Trans Isomer 1; 3-{[3-hydroxybutan-2-yl]oxy}-5-(5-methyl-1,3-thiazol-2-yl)-N-{(1R)-1-[6-(trifluoromethyl)pyridazin-3-yl]ethyl}benzamide;
Trans Isomer 1; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide;
Cis Isomer 1; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide;
Cis Isomer 2; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide;
Trans Isomer 2; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide;
Cis Isomer 1; 3-[(-3-hydroxybutan-2-yl)oxy]-5-(5-methyl-1,3-thiazol-2-yl)-N-{(1R)-1-[6-(trifluoromethyl)pyridazin-3-yl]ethyl}benzamide;
Cis Isomer 2; 3-[(-3-hydroxybutan-2-yl)oxy]-5-(5-methyl-1,3-thiazol-2-yl)-N-{(1R)-1-[6-(trifluoromethyl)pyridazin-3-yl]ethyl}benzamide;
Cis Isomer 1; 3-[(3-hydroxybutan-2-yl)oxy]-5-(5-methyl-1,3-thiazol-2-yl)-N-{(1R)-1-[2-(trifluoromethyl)pyrimidin-5-yl]ethyl}benzamide;
Cis Isomer 2; 3-[(3-hydroxybutan-2-yl)oxy]-5-(5-methyl-1,3-thiazol-2-yl)-N-{(1R)-1-[2-(trifluoromethyl)pyrimidin-5-yl]ethyl}benzamide
   for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.
   An even more preferred embodiment of the present invention is the use of Cis Isomer 1; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide for the treatment or prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.
   It is to be understood that the present invention relates also to the use of any combination of the preferred embodiments described above.

The synthesis of compounds of general formula (I) is described in WO2016/091776.

Pharmaceutical Compositions of the Compounds of the Invention

This invention also relates to the use of pharmaceutical compositions containing one or more compounds of the general formula (I). These compositions can be utilised to achieve the desired pharmacological effect by administration to a patient in need thereof. A patient, for the purpose of the present invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. A compound of general formula (I) can be administered with pharmaceutically acceptable carriers well known in the art using any effective conventional dosage unit forms, including immediate, slow and timed release preparations, orally, parenterally, topically, inhaled, nasally, sublingually, intravescially, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known in the art for the manufacture of pharmaceutical compositions. The solid unit dosage forms can be a capsule that can be of the ordinary hard- or soft-shelled gelatine type containing, for example, surfactants, lubricants, and inert fillers such as lactose, sucrose, calcium phosphate, and corn starch.

In another embodiment, a compound of general formula (I) may be tableted with conventional tablet bases such as lactose, sucrose and cornstarch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavouring and colouring agents described above, may also be present.

The pharmaceutical compositions containing a compound of general formula (I) may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more colouring agents; one or more flavouring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, such as methyl and propyl parabens and flavouring and colouring agents.

A compound of general formula (I) may also be administered parenterally, that is, subcutaneously, intravenously, intravescially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of the present invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxylpropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived from a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition containing a compound of general formula (I) may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of a compound of general formula (I) in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

A composition containing a compound of general formula (I) may also be administered in the form of controlled release formulations for parenteral and intravesical administration include liposomal, polymeric microsphere and polymeric gel formulations which are known in the art.

A composition containing a compound of general formula (I) may also be administered in the form of prolonged release, implant, or depot formulation.

Another formulation employed in the methods of the present invention employs an aerosol, which enables the delivery of the compound of the general formula (I) to the airways with minimal systemic drug exposure. There are several aerosol formulations known, which can be used for that purpose, like for example liquid or dry particles generated by nebulizers or dry powder inhalers.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions containing a compound of general formula (I) can also contain other conventional pharmaceutically acceptable compounding ingredients, generally referred to as carriers or diluents, as necessary or desired. Conventional procedures for preparing such compositions in appropriate dosage forms can be utilized.

Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)—Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine); adsorbents (examples include but are not limited to powdered cellulose and activated charcoal);

aerosol propellants (examples include but are not limited to carbon dioxide, CCl₂F₂, F₂ClC—CClF₂ and CClF₃);

air displacement agents (examples include but are not limited to nitrogen and argon);

antifungal preservatives (examples include but are not limited to benzoic acid, butylparaben, ethylparaben, methylparaben, propylparaben, sodium benzoate); antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate);

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);

chelating agents (examples include but are not limited to edetate disodium and edetic acid);

colourants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite); emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate);

flavourants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose); tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and wetting agents (examples include but are not limited to heptadecaethylene oxycetanol, lecithins, sorbitol monooleate, polyoxyethylene sorbitol monooleate, and polyoxyethylene stearate).

Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

A compound of general formula (I) can be administered as the sole pharmaceutical agent or in combination with one or more other pharmaceutical agents where the combination causes no unacceptable adverse effects. The present invention relates also to the use of such combinations containing a compound of general formula (I) for the use in treating and/or for prophylaxis of diseases or disorders which are associated with nerve fibre sensitization, in particular for the treatment and prophylaxis of Chronic Cough (CC), Idiopathic Pulmonary Fibrosis (IPF), Chronic Obstructive Pulmonary Disorder (COPD), and asthma.

A compound of general formula (I) can be combined with therapeutic agents or active ingredients, that are already approved or that are still under development for the treatment and/or prophylaxis of diseases, which are related to or mediated by P2X3 receptor.

For the treatment and/or prophylaxis of Chronic Cough and symptoms related to Chronic Cough, as well as for the treatment and/or prophylaxis of Idiopathic Pulmonary Fibrosis (IPF), Chronic Obstructive Pulmonary Disorder (COPD), and asthma, a compound of general formula (I) can be administered in combination or as comedication with cough suppressants like dextromethorphan, benzonatate, codeine or hydrocodone; neuromodulators such as gabapentine, pregabalin, amitriptyline, baclofen, morphine, with inhalative agents to treat eosinophilic bronchitis, COPD or asthma like budesonide, beclomethasone, fluticasone, theophylline, ipatropiumbromid, montelukast or salbutamol; with drugs like proton pump inhibitors which are used to treat acid reflux, for example omeprazole, esomeprazole, lansoprazole, ranitidine, famotidine, cimetidine; and promotility agents such as metoclopramide; with nasal or topical glucocorticoids like fluticasone or mometasone or triamcinolone; or with oral antihistamines like loratadine, fexofenadine or cetirizine.

Methods of Treating

The present invention relates to a method for using a compound of general formula (I) and compositions thereof, to inhibit the P2X3 receptor and therefore achieve an efficacious treatment of Chronic Cough, Idiopathic Pulmonary Fibrosis (IPF), Chronic Obstructive Pulmonary Disorder (COPD), and asthma.

The present invention also provides a method for using a compound of general formula (I) and compositions thereof, to selectively inhibit the P2X3 receptor over the P2X2/3 receptor which means at least 3-fold selectivity over the P2X2/3 receptor. The advantage of having such selective compounds is the access to a method of treating Chronic Cough, Idiopathic Pulmonary Fibrosis (IPF), Chronic Obstructive Pulmonary Disorder (COPD), and asthma with less or no effect to the taste sensitivity of a patient. This provides the advantage of having an effective treatment for Chronic Cough, Idiopathic Pulmonary Fibrosis (IPF), Chronic Obstructive Pulmonary Disorder (COPD), and asthma available with fewer side effects like physical dependency, increased heart rate, xerostomia, constipation, nausea, drowsiness, or sedation, which may be used as chronic treatment if necessary.

Therefore, the quality of life of patients with Chronic Cough, Idiopathic Pulmonary Fibrosis (IPF), Chronic Obstructive Pulmonary Disorder (COPD), and asthma, may be highly improved.

A compound of general formula (I) is also useful in a method for selectively inhibiting the P2X3 receptor over the P2X2/3 receptor with at least 10-fold selectivity over the P2X2/3 receptor. In addition to that, the present invention also provides a method of treating mammalian including human disorders and diseases, i.e. Chronic Cough, Idiopathic Pulmonary Fibrosis (IPF), Chronic Obstructive Pulmonary Disorder (COPD), and asthma, with a compound of formula (I).

These disorders have been well characterized in humans, but also exist with a similar etiology in other mammals, and can be treated by administering pharmaceutical compositions containing a compound of general formula (I.)

Dose and Administration

Based upon standard laboratory techniques known to evaluate compounds useful for the treatment of disorders and/or disease which are mediated by the P2X3 receptor, by standard toxicity tests and by standard pharmacological assays for the determination of treatment of the conditions identified above in mammals, and by comparison of these results with the results of known medicaments that are used to treat these conditions, the effective dosage of a compound of general formula (I) can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, preferably from about 0.01 mg/kg to about 50 mg/kg body weight per day. A preferred administration of present compounds includes but is not limited to 0.01 mg/kg to about 6 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 400 mg of active ingredient, and can be administered one or more times per day or less than once a day. A preferred oral unit dosage for an administration of a compound of general formula (I) includes but is not limited to 0.5 mg to about 400 mg one to three times a day to once a week. The average daily dosage for administration by injection, including intravenous, intravesical, intramuscular, subcutaneous, and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of general formula (I) or a pharmaceutically acceptable salt or ester or composition thereof can be ascertained by those skilled in the art using conventional treatment tests.

Methods of testing for a particular pharmacological or pharmaceutical property are well known to persons skilled in the art.

The example testing experiments described herein serve to illustrate the present invention and the invention is not limited to the examples given.

Biological Assays

Examples were tested in selected biological assays one or more times. Unless stated otherwise, when tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

Data of compounds of formula (I) obtained from intracellular calcium measurement to assess antagonist activity at human P2X3 and human P2X2/3 receptors are described in WO2016/091776.

Vagal Depolarization—Inhibition of Human Vagus Nerve by P2X3 Inhibitors

The aim of the study is to test the effects of P2X3 antagonists on the deoplarisation of human vagus nerve as an ex vivo measure of blocking Chronic Cough. Human vagal tissue was dissected from 4 human lungs (obtained from healthy tissue donors).

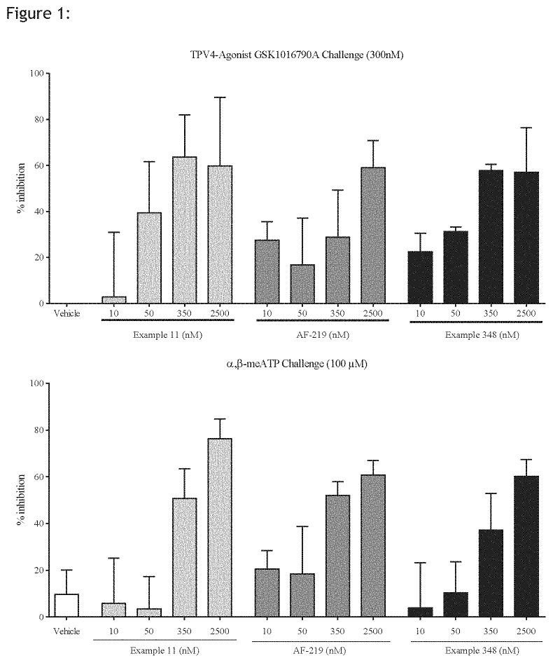

The tissue was assayed using an O₂/CO₂ gassed, grease-gap recording system as described in Birrell et al, Am J Respir Crit Care Med. 2009, 180:1042-1047; Bonvini et al, J Allergy Clin Immunol. 2016, 138 (1), 249-261, Bonvini et al, Naunyn Schmiedebergs Arch Pharmacol. 2015, 388: 401-420. Briefly, after the tissue had stabilised it was exposed to two 2 minute challenges with a sub-maximal concentration of agonists (TRPV4 agonist GSK 1016790A, 300 nM). Sub-maximal concentration means a concentration where no full efficacy is observed, i.e. stimulation with the agonist within the dynamic range. Typical values are 80% of the maximal efficacy that can be achieved. The tissue was then exposed to vehicle (0.1 Vol % DMSO solution in Krebs buffer) or test compound for 10 minutes and then the tissue was re-challenged with agonist in the presence of the test compound. Following a wash step with Krebs buffer, the vagus tissue was re-challenged with the agonist to demonstrate recovery of the response/tissue viability. Only one experiment was performed on each piece of tissue and n=4 means tissue from 4 different lungs/guinea pigs. The data was electrophysiologically recorded as actual depolarisation levels and presented as mean percentage inhibition. Results can be found in FIG. 1. Both compounds (examples 11 and 348 as described in WO2016/091776) showed dose-dependent inhibition of currents. Importantly, the effect size observed was the same for both examples and AF-219 demonstrating that P2X3-homomer selective compounds such as examples 11 and 348 have the same efficacy in blocking vagus nerve depolarization as a P2X3 and P2X2/3 non-selective compound such as AF-219.

Taste Assessment

Taste is the sensation produced when nutrients and other compounds in the mouth react chemically with taste receptor cells located on taste buds. Taste buds are aggregates of 50-100 polarized neuroepithelial cells and express multiple receptors known for transmission of taste stimuli, in case of the bitter taste there are 50 different taste receptors described. Five recognized taste qualities—sweet, sour, bitter, salty, and umami are detected by taste buds. Numerous drugs have the potential to adversely influence a patient's sense of taste, either by decreasing function or producing perceptual distortions or phantom tastes. In case of suspected, drug mediated disturbance of taste perception, a quantitative assessment of gustatory sensitivity is needed to evaluate a drug exposure effect relationship in a clinical setting.

The Taste Strips (Burghart Messtechnik GmbH) are a validated test method for ascertaining tasting performance (ISO certified). The following concentrations have been applied on the taste strips: sweet: 0.4, 0.2, 0.1, 0.05 g/ml sucrose; sour: 0.3, 0.165, 0.09, 0.05 g/ml citric acid; salty: 0.25, 0.1, 0.04, 0.016 g/ml sodium chloride; bitter: 0.006, 0.0024, 0.0009, 0.0004 g/ml quinine hydrochloride. The Strips were used to check the tasting performance of the whole mouth by placing a Taste Strip on the tongue and closing the mouth (the subject can move the tongue). Before presenting randomized taste strips to subject, the subject had received four taste strips soaked with the second highest concentration of one of the four compounds and a control strip each to experience the quality of sweet, sour, salt, bitter and the paper itself. Afterwards and between the 4 taste strips the mouth was rinsed with a sip of tap water according to individual needs. After applying the initial reference taste strips subjects had to wait at least 10 min before the actual time was recorded and randomized testing started. The taste strips were presented in a pseudo-randomized order (increasing concentration for each quality) and placed on the tongue. The subject's task was to choose one of five possible answers on a form (sweet, sour, salty, bitter, no taste). Before and after each testing, the mouth was rinsed with a sip of tap water according to individual needs. The lowest concentrations of each taste quality should have been identified by half of the healthy subjects only; the highest concentration should have been identified by approximately 100% of the subjects.

The taste strips were tested under blinded conditions to the subject. The number of correctly identified taste strips per subjects was counted with the maximum number achievable being 18 points (4 concentration/taste quality plus to blank paper strips).

Taste Related Adverse Events:

Taste related adverse events describe adverse events that lead to a change in taste perception, e.g. dysgeusia (distortion of taste), hypogeusia (decrease in taste sensitivity) and ageusia (complete lack of taste).

Adverse events (AE) in the context of a clinical study are defined as any untoward medical occurrence (i.e. any unfavorable and unintended sign including abnormal laboratory findings, symptom or disease) in a patient or clinical investigation subject after providing written informed consent for participation in the study. Therefore, an AE may or may not be temporally or causally associated with the use of a medicinal (investigational) product.

Taste related adverse events were recorded and taste assessment tests as described above were applied during clinical studies with 3-(5-Methyl-1,3-thiazol-2-yl)-5-[(3R)-tetrahydrofuran-3-yloxy]-N-{(1R)-1-[2-(trifluoromethyl)pyrimidin-5-yl]ethyl}benzamide as well as during clinical studies with 3-{[(2R)-4-methylmorpholin-2-yl]methoxy}-5-(5-methyl-1,3-thiazol-2-yl)-N-{(1R)-1-[2-(trifluoromethyl)-pyrimidin-5-yl]ethyl}benzamide, in humans.

3-(5-Methyl-1,3-thiazol-2-yl)-5-[(3R)-tetrahydrofuran-3-yloxy]-N-{(1R)-1-[2-(trifluoromethyl)pyrimidin-5-yl]ethyl}benzamide has been administered as single oral dose with increasing amounts starting from 10 mg per subject to 800 mg per subject in comparison with placebo to healthy young males.

No taste related adverse were observed. In addition, no relevant effects on taste perception were observed based on the taste assessment test. Taste tests were conducted at baseline (i.e. 24 hrs prior to first drug administration), 3 hrs after drug administration and 6 days after drug administration. Mean overall taste score in the different treatment groups at baseline ranged between 11.2 and 14.8 points, which represents normal values for the investigated population of healthy young males. Under treatment no significant or relevant changes in taste scores were observed. Changes from baseline in the taste scores ranged between −1.7 points to +2.0 points and thus varied around zero and did not show any difference between actively and placebo treated subjects. No statistical significant changes were found when a non-parametric test was applied.

Investigations on the Efficacy of Example Compounds

### 3-(5-Methyl-1,3-thiazol-2-yl)-5-[(3R)-tetrahydrofuran-3-yloxy]-N-{(1R)-1-[2-(tri-fluoromethyl)pyrimidin-5-yl]ethyl}benzamide

In a first part of a two-part, double-blind, placebo-controlled, randomized, parallel-group study safety and tolerability of ascending repeated oral doses of the drug to be tested are assessed in 47 healthy male volunteers, followed by a second two-way crossover administration of four different doses in 40 patients with refractory chronic cough to assess safety, tolerability and efficacy for proof of concept.

In this study, the effect of the drug on cough is measured using an electronic 24 hour cough monitor. The device is stored in a pouch worn around the waist or attached to the belt of the subject. There is one microphone and one chest sensor connected to the cough monitor. The microphone is clipped to the clothes of the subject and the sensor is put on the chest with a sticker. The device records the number of coughs produced during a 24 hour monitoring period and is removed once the recording period is finished. The monitor is worn for 24 hours—including during the night. At the end of the 24 hours, the monitor is automatically stopped. The cough monitor records not only cough sounds, but also other background sounds including the own conversation and those of people around the subject, particularly if they are very close. The recordings made by the cough monitor is then analysed by technicians who count the number of coughs.

In the first part of the above-described protocol, 3-(5-Methyl-1,3-thiazol-2-yl)-5-[(3R)-tetrahydrofuran-3-yloxy]-N-{(1R)-1-[2-(trifluoromethyl)pyrimidin-5-yl]ethyl}-benzamide has been administered as multiple oral dose with increasing amounts starting from 10 mg per subject twice daily to 750 mg per subject twice daily in comparison with placebo to healthy young males. At each of the dose groups (10 mg, 50 mg, 200 mg and 750 mg) 9 subjects were treated with active drug while 3 subjects were treated with placebo with the exception of the 750 mg dose group where only 8 subjects were treated with active drug. The treatment duration was 14 days. 3-(5-Methyl-1,3-thiazol-2-yl)-5-[(3R)-tetrahydrofuran-3-yloxy]-N-{(1R)-1-[2-(tri-fluoromethyl)pyrimidin-5-yl]ethyl}benzamide had an influence on perception of taste in few subjects only based on spontaneously reported adverse events (AE).

The frequency of those taste AEs was evenly distributed between subjects receiving active treatment (3 subjects; 8.6%) and subjects receiving placebo (1 subject; 8.3%). These AEs occurred mostly only once during treatment period, were short-lasting and transient AEs (1 subject treated with 200 mg 3-(5-Methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide—AE duration of 2 hours on day 11; one subject treated with 750 mg 3-(5-Methyl-1,3-thiazol-2-yl)-5-[(3R)-tetrahydrofuran-3-yloxy]-N-{(1R)-1-[2-(tri-fluoromethyl)pyrimidin-5-yl]ethyl}benzamide—AE duration of 0.5 hours on day 3; one subject treated with placebo—AE duration of 1.5 hours on day 11; only in one subject treated with 200 mg 3-(5-Methyl-1,3-thiazol-2-yl)-5-[(3R)-tetrahydrofuran-3-yloxy]-N-{(1R)-1-[2-(trifluoro-methyl)pyrimidin-5-yl]ethyl}benzamide), the change in taste perception was repeatedly occurring during continued treatment starting on treatment day 4 and lasting until 5 days after end of treatment.

In addition to the spontaneous AE reporting, no relevant effects on taste perception were observed based on the taste assessment test.

In comparison to known treatment approaches for Chronic Cough as well as for other chronic upper respiratory diseases like Idiopathic Pulmonary Fibrosis (IPF), Chronic Obstructive Pulmonary Disorder (COPD), and asthma with ACE inhibitors, beta blockers or cortisone, which have undesired side effect profiles like physical dependency, increased heart rate, xerostomia, constipation, nausea, drowsiness or sedation, no such adverse events (physical dependency, increased heart rate, xerostomia, constipation, drowsiness and sedation) were observed in the above-mentioned multiple dose study with the exception of one case of nausea at the 50 mg dose.

### 3-{[(2R)-4-methymorpholin-2-yl]methoxy}-5-(5-methyl-1,3-thiazol-2-yl)-N-{(1R)-1-[2-(trifluoromethyl)-pyrimidin-5-yl]ethyl}benzamide

In a first part of a two-part, double-blind, placebo-controlled, randomized, parallel-group study, the safety and tolerability of ascending repeated oral doses of the drug to be tested are assessed in 36 healthy male volunteers, followed by a second part with a two-way crossover administration of four different doses in patients with refractory chronic cough to assess safety, tolerability and efficacy for proof of concept.

In this study, the effect of the drug on cough is measured using an electronic 24 hour cough monitor. The device is stored in a pouch worn around the waist or attached to the belt of the subject. There is one microphone and one chest sensor connected to the cough monitor. The microphone is clipped to the clothes of the subject and the sensor is put on the chest with a sticker. The device records the number of coughs produced during a 24 hour monitoring period and is removed once the recording period is finished. The monitor is worn for 24 hours—including during the night. At the end of the 24 hours, the monitor is automatically stopped. The cough monitor records not only cough sounds, but also other background sounds including the own conversation and those of people around the subject, particularly if they are very close. The recordings made by the cough monitor is then analysed by technicians who count the number of coughs.

In the first part of the above-described protocol, 3-{[(2R)-4-methylmorpholin-2-yl]methoxy}-5-(5-methyl-1,3-thiazol-2-yl)-N-{(1R)-1-[2-(trifluoromethyl)-pyrimidin-5-yl]ethyl}benzamide has been administered as multiple oral dose with increasing amounts starting from 20 mg per subject twice daily to 250 mg per subject twice daily in comparison with placebo to healthy young males. At each of the dose groups 9 subjects were treated with active drug while 3 subjects were treated with placebo. The treatment duration was 14 days.

Only a few taste related adverse events were reported as dysgeusia in the dose groups expected to be therapeutic (20 mg and 80 mg). One subject each at the dose groups 20 mg and 80 mg reported a taste related adverse event. These AE occurred only once during the treatment period, were short-lasting and transient, i.e. resolved under continuous treatment (AE in the 20 mg dose group, one subject, starting on day 4 about 90 minutes after the morning dose and lasting for 1 hour; AE in the 80 mg dose group, one subject, starting on day 10 about 12 hours after the morning dose and lasting for about 2 hours).

In addition to the spontaneous AE reporting, no relevant effects on taste perception were observed based on the taste assessment test.

In comparison to known treatment approaches for Chronic Cough as well as for other chronic upper respiratory diseases like Idiopathic Pulmonary Fibrosis (IPF), Chronic Obstructive Pulmonary Disorder (COPD), and asthma with ACE inhibitors, beta blockers or cortisone, which have undesired side effect profiles like physical dependency, increased heart rate, xerostomia, constipation, nausea, drowsiness or sedation, no such adverse events (physical dependency, increased heart rate, constipation, drowsiness and sedation) were observed in the above-mentioned multiple dose study at expected therapeutic doses of 20 mg and 80 mg with the exception of one case of nausea at the 20 mg dose. This AE have been resolved after treatment.

No relevant effects on taste perception in the context of the present invention mean that no effect on taste perception has been observed in at least 80% of subjects under treatment according to the above-described protocol over a period of 14 days.

1: A method for treatment or prophylaxis of a disease or a disorder associated with nerve fibre sensitization in a subject in need thereof, in particular for the treatment and prophylaxis of Chronic Cough (CC), Idiopathic Pulmonary Fibrosis (IPF), Chronic Obstructive Pulmonary Disorder (COPD), and asthma, comprising administering to the subject an effective amount of a compound of formula (I): 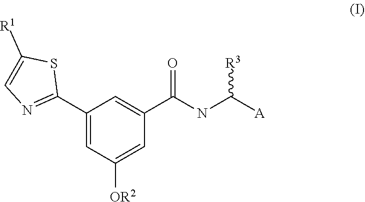
wherein
R¹ is a halogen atom, C₁-C₄-alkyl or C₃-C₆-cycloalkyl,
wherein said C₁-C₄-alkyl is optionally substituted with 1-5 halogen atoms which are the same or different;
R² is —C₂-C₆-alkyl-OR⁴, —(CH₂)_{q}—(C₃-C₇-cycloalkyl), —(CH₂)_{q}-(6- to 12-membered heterobicycloalkyl), —(CH₂)_{q}-(4- to 7-membered heterocycloalkyl), —(CH₂)_{q}-(5- to 10-membered heteroaryl) or —C₂-C₆-alkynyl,
wherein said —(CH₂)_{q}—(C₃-C₇-cycloalkyl), —(CH₂)_{q}-(6- to 12-membered heterobicycloalkyl) and —(CH₂)_{q}-(4- to 7-membered heterocycloalkyl) are optionally substituted with one or more substituents which are the same or different, at any ring carbon atom and selected from the group consisting of C₁-C₄ alkyl, optionally substituted with 1-5 halogen atoms which are the same or different, a halogen atom, —NR^{a}R^{b}, COOR⁵ and oxo (═O),
wherein any ring nitrogen atom, if present in said —(CH₂)_{q}-(6- to 12-membered heterobicycloalkyl) and —(CH₂)_{q}-(4- to 7-membered heterocycloalkyl) is independently substituted with R′; and
wherein said —(CH₂)_{q}-(5- to 10-membered heteroaryl) is optionally substituted with one or more substituents which are the same or different, and selected from the group consisting of C₁-C₄-alkyl optionally substituted with 1-5 halogen atoms which are the same or different, a halogen atom, —NR^{a}R^{b} and —COOR⁵;
R³ is hydrogen or C₁-C₄-alkyl optionally substituted with 1-5 halogen atoms which are the same or different;
R⁴ and R⁵ are independently hydrogen or C₁-C₄-alkyl;
R^{a} and R^{b} are independently hydrogen or C₁-C₄-alkyl;
R^{c} is hydrogen, C₁-C₄-alkyl optionally substituted with 1-5 halogen atoms which are the same or different, —C(O)O—C₁-C₄-alkyl, or —C(O)—C₁-C₄-alkyl;
A is 5- to 10-membered heteroaryl which is optionally substituted with one or more substituents, which are the same or different, and selected from the group consisting of a halogen atom, C₁-C₃-alkyl, and C₁-C₃-alkoxy,
wherein said C₁-C₃-alkyl and C₁-C₃-alkoxy are optionally substituted with 1-5 halogen atoms which are the same or different; and
q represents an integer of 0, 1, or 2,
or an isomer, enantiomer, diastereomer, racemate, hydrate, solvate, or salt thereof, or a mixture of any of the foregoing.

2: The method according to claim 1, wherein the compounds have general formula (Ia): 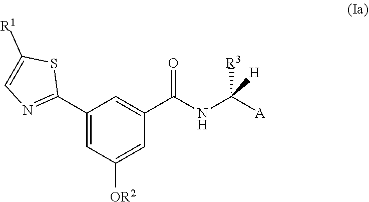
wherein A, R¹, R² and R³ have the meanings are as defined in claim 1
or an isomer, enantiomer, diastereomer, racemate, hydrate, solvate, or salt thereof, or a mixture of any of the foregoing.

3: The method according to claim 1, wherein the compound is
3-(5-methyl-1,3-thiazol-2-yl)-5-[(3R)-tetrahydrofuran-3-yloxy]-N-{(1R)-1-[2-(trifluoromethyl)pyrimidin-5-yl]ethyl}benzamide; or
3-(5-methyl-1,3-thiazol-2-yl)-5-[(3S)-tetrahydrofuran-3-yloxy]-N-{(1R)-1-[2-(trifluoromethyl)pyrimidin-5-yl]ethyl}benzamide;
or a hydrate, solvate, or salt thereof, or a mixture of any of the foregoing.

4: The method according to claim 3, wherein the compound is 3-(5-methyl-1,3-thiazol-2-yl)-5-[(3R)-tetrahydrofuran-3-yloxy]-N-{(1R)-1-[2-(trifluoromethyl)pyrimidin-5-yl]ethyl}benzamide or a hydrate, solvate, or salt thereof, or a mixture of any of the foregoing.

5: The method according to claim 1 wherein the compound is
3-{[(2S)-4-methylmorpholin-2-yl]methoxy}-5-(5-methyl-1,3-thiazol-2-yl)-N-{(1R)-1-[2-(trifluoromethyl)pyrimidin-5-yl]ethyl}benzamide; or
3-{[(2R)-4-methylmorpholin-2-yl]methoxy}-5-(5-methyl-1,3-thiazol-2-yl)-N-{(1R)-1-[2-(trifluoromethyl)-pyrimidin-5-yl]ethyl}benzamide;
or a hydrate, solvate, or salt thereof, or a mixture of any of the foregoing.

6: The method according to claim 5, namely wherein the compound is 3-{[(2R)-4-methylmorpholin-2-yl]methoxy}-5-(5-methyl-1,3-thiazol-2-yl)-N-{(1R)-1-[2-(trifluoromethyl)-pyrimidin-5-yl]ethyl}benzamide or a hydrate, solvate, or salt thereof, or a mixture of any of the foregoing.

7: A method for treatment or prophylaxis of a disease or a disorder associated with nerve fibre sensitization in a subject in need thereof, in particular for the treatment and prophylaxis of Chronic Cough (CC), Idiopathic Pulmonary Fibrosis (IPF), Chronic Obstructive Pulmonary Disorder (COPD), and asthma, comprising administering to the subject an effective amount of a pharmaceutical composition comprising a compound of formula (I): 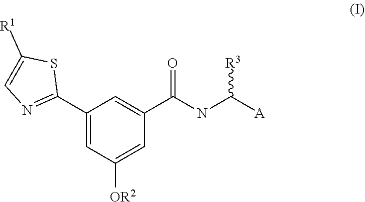
wherein
R¹ is a halogen atom, C₁-C₄-alkyl or C₃-C₆-cycloalkyl,
wherein said C₁-C₄-alkyl is optionally substituted with 1-5 halogen atoms which are the same or different:
R² is —C₂-C₆-alkyl-OR⁴, —(CH₂)_{q}—(C₃-C₇-cycloalkyl), —(CH₂)_{q}-(6- to 12-membered heterobicycloalkyl), —(CH₂)_{q}-(4- to 7-membered heterocycloalkyl), —(CH₂)_{q}-(5- to 10-membered heteroaryl) or —C₂-C₆-alkynyl,
wherein said —(CH₂)_{q}—(C₃-C₇-cycloalkyl), —(CH₂)_{q}-(6- to 12-membered heterobicycloalkyl) and —(CH₂)_{q}-(4- to 7-membered heterocycloalkyl) are optionally substituted with one or more substituents which are the same or different, at any ring carbon atom and selected from the group consisting of C₁-C₄ alkyl, optionally substituted with 1-5 halogen atoms which are the same or different, a halogen atom, —NR^{a}R^{b}, COOR⁵, and oxo (═O),
wherein any ring nitrogen atom, if present in said —(CH₂)_{q}-(6- to 12-membered heterobicycloalkyl) and —(CH₂)_{q}-(4- to 7-membered heterocycloalkyl) is independently substituted with R′; and
wherein said —(CH₂)_{q}-(5- to 10-membered heteroaryl) is optionally substituted with one or more substituents which are the same or different, and selected from the group consisting of C₁-C₄-alkyl optionally substituted with 1-5 halogen atoms which are the same or different, a halogen atom, —NR^{a}R^{b} and —COOR⁵:
R³ is hydrogen or C₁-C₄-alkyl optionally substituted with 1-5 halogen atoms which are the same or different;
R⁴ and R⁵ are independently hydrogen or C₁-C₄-alkyl;
R^{a} and R^{b} are independently hydrogen or C₁-C₄-alkyl;
R^{c} is hydrogen, C₁-C₄-alkyl optionally substituted with 1-5 halogen atoms which are the same or different, —C(O)O—C₁-C₄-alkyl, or —C(O)—C₁-C₄-alkyl;
A is 5- to 10-membered heteroaryl which is optionally substituted with one or more substituents, which are the same or different, and selected from the group consisting of a halogen atom, C₁-C₃-alkyl, and C₁-C₃-alkoxy,
wherein said C₁-C₃-alkyl and C₁-C₃-alkoxy are optionally substituted with 1-5 halogen atoms which are the same or different; and
q represents an integer of 0, 1, or 2,
or an isomer, enantiomer, diastereomer, racemate, hydrate, solvate, or salt thereof, or a mixture of any of the foregoing, and a pharmaceutically acceptable diluent or carrier.

8: The method according to claim 1, wherein the method is a method of long-term treatment.

9: The method according to claim 1, wherein the method is a method of oral treatment.

10: The method according to claim 1 the method is for the treatment or prophylaxis of Chronic Cough (CC).

11: The method according to claim 1 the method is for the treatment and prophylaxis Refractory or Unexplained Chronic Cough (RUCC).

12: The method according to claim 10 the method is for the treatment of Chronic Cough (CC) and the compound is 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide or a hydrate, solvate, or salt thereof, or a mixture of any of the foregoing.

13: The method according to claim 11 the method is for the treatment of Refractory or Unexplained Chronic Cough (RUCC) and the compound is 3-(5-methyl-1,3-thiazol-2-yl)-5-[(3R)-tetrahydrofuran-3-yloxy]-N-{(1R)-1-[2-(trifluoromethyl)-pyrimidin-5-yl]ethyl}benzamide or a hydrate, solvate, or salt thereof, or a mixture of any of the foregoing.

14: The method according to claim 10 the method is for the treatment of Chronic Cough (CC) and the compound is 3-{[(2R)-4-methylmorpholin-2-yl]methoxy}-5-(5-methyl-1,3-thiazol-2-yl)-N-{(1R)-1-[2-(trifluoromethyl)-pyrimidin-5-yl]ethyl}benzamide or a hydrate, solvate, or salt thereof, or a mixture of any of the foregoing.

15: The method according to claim 11 the method is for the treatment of Refractory or Unexplained Chronic Cough (RUCC) and the compound is 3-{[(2R)-4-methylmorpholin-2-yl]methoxy}-5-(5-methyl-1,3-thiazol-2-yl)-N-{(1R)-1-[2-(trifluoromethyl)-pyrimidin-5-yl]ethyl}benzamide or a hydrate, solvate, or salt thereof, or a mixture of any of the foregoing.

16: The method according to claim 12, wherein the method is a method of long-term treatment or prophylaxis.

17: The method according to claim 12, wherein the method is a method of oral treatment or prophylaxis.

18: The method according to claim 12, wherein the method is a method of long-term and oral treatment or prophylaxis.
